(19) United States
(12) Patent Application Publication
(10) Pub. No.: US 2009/0221038 A1
(43) Pub. Date: Sep. 3, 2009

(54) Twin-Arginine Translocation (TAT) Streptomyces Signal Sequences

(76) Inventors: Tracy Palmer, Norfolk (GB); David Andrew Widdick, Norfolk (GB)

Correspondence Address: Elena E. Quertermous;Genencor International, 925 Page Mill Road, Palo Alto, CA, 94304-1013, US

(21) Appl. No.: 12/086,836

(22) PCT Filed: Dec. 20, 2006
(86) PCT No.: PCT/GB2006/004816

§ 371 Date: Jun. 19, 2008

Related U.S. Application Data
(60) Provisional application No. 60/751,872, filed on Dec. 20, 2005.

(51) Int. Cl. C12P 21/02; C07K 14/195; C12N 15/11; C12N 15/00; C12N 9/96; C12N 1/21

(52) U.S. Cl. 435/69.7; 530/350; 536/23.7; 435/320.1; 435/188; 435/252.3

(57) ABSTRACT

Described herein are novel Tat signal polypeptides and methods for using the Tat signal polypeptides for producing heterologous polypeptides. A novel reporter assay for testing the biological activity of the secreted proteins is also described.

### FIELD OF THE INVENTION

This invention relates to Twin-arginine translocation (Tat) signal peptides, which have been identified in the cell wall associated fraction of the gram-positive soil bacterium Streptomyces coelicolor. The invention further relates to fusion polypeptides comprising a TAT signal peptide and a heterologous polypeptide.

### BACKGROUND

In prokaryotes two pathways for protein translocation across the cytoplasmic membrane have been recognized. In most bacteria the general secretory (Sec) pathway is the best-characterized route for protein export. Proteins exported by this pathway are translocated across the membrane in an unfolded state through a membrane-embedded translocon to which they are targeted by cleavable N-terminal signal peptides (Mod et al., (2001) Trends in Microbiology 9:494-500). More recently a second general export pathway has been described, which is designated the twin-arginine translocation (Tat) pathway and reference is made to US 2002/0110860; WO 03/079007; Berks, B. C. (1996) Mol. Microbiol. 22:393-404 and Tjalsma et al., (2000) Microbiol. & Molecul. Bio. Reviews 64:515-547. Unlike the Sec system, the Tat system is involved in the transport of pre-folded protein substrates (Thomas et al., (2001) Mol. Microbiol 39:47-53).

Proteins are targeted to the Tat pathway by possession of N-terminal tripartite signal peptides. The signal peptides include a conserved twin-arginine motif in the N-region of Tat signal peptide. The motif has been defined as R-R-x-φ-φ, where φ represents a hydrophobic amino acid. In E. coli the Tat pathway comprises the three-membrane protein TatA, TatB and TatC. A fourth protein TatE forms a minor component of the Tat machinery and has a similar function to TatA. Studies by several groups suggest that the major role of the Tat pathway is in the translocation of redox proteins that integrate their cofactors in the cytoplasm. Other more recent studies indicate that the Tat pathway may play a broader role in protein secretion (Ochsner et al., (2001) Proc. Natl. Acad. Sci. USA 99:8312-8317). Because of the ability to secrete pre-folded protein substrates, the Tat pathway represents a significant mechanism for secreting a high level of heterologous proteins.

Estimates of Tat substrates in organisms other than Bacillus subtilits and E. coli have been based predominantly in in silico analysis of genome sequences using programs trained to recognize specific features of tat targeting sequences. While these programs are useful tools for identifying candidate Tat substrates encoded within bacterial genomes experimental verification of the in silico predicted sequences other than phenotype analysis has been lacking.

Many of these predicted Tat substrates are in microorganisms of the Streptomyces genes such as S. coelicolor and S. avermitilis. Streptomyces are gram-positive spore forming microorganisms which produce a range of diverse and important secondary metabolites, including many commercially available antibiotics. Streptomyces are important in the field of biotechnology because they are prolific protein secretors.

Prior in silico predictions estimate between 145 and 189 proteins from S. coelicolor may be Tat dependent. The present invention demonstrates the verification and importance of the Tat secretory pathway in Streptomyces and further is directed to Tat signal peptides which comprise a motif outside of the previous identified Tat signal peptide motif. Further the Tat signal peptides according to the invention maybe useful in the secretion of heterologous proteins in Streptomyces.

### SUMMARY OF THE INVENTION

Provided herein are novel Tat signal peptides and methods of using the novel Tat signal peptides for producing heterologous polypeptides in a bacterial host cell as described in the appended claims.

In a first aspect, the invention is directed to novel Twin Arginine Translocation (TAT) signal peptides.

In one aspect, the invention is directed to an isolated signal peptide comprising the TAT signal sequence motif (X⁻¹)RR(X⁺²)(X⁺³)(X⁺⁴), wherein R is arginine, X⁻¹ is amino acid M, H, A, P, K, R, N, T, G, S, D, Q or E; X⁺² is amino acid A, P, K, R, N, T, G, S, D, Q or E; X⁺³ is I, W, F, L, V, Y, M, C, H, A, P, N or T; and X⁺⁴ is Q, I, L, V, M or F, and wherein said motif is not within the first 35N′ terminal residues of the amino acid sequence of the polypeptide.

In another aspect, the invention is directed to an isolated TAT signal peptide comprising the sequence motif (X⁻¹)RR(X⁺²)(X⁺³)(X⁺⁴), wherein R is arginine, X⁻¹ is amino acid H, A, P, K, R, N, T, G, S, D, Q E or L; X⁺² is A, P, K, R, N, T, G, S, C), Q or E; X⁺³ is I, W, F, L, V, Y, M, C, H, A, P, N or T; and X⁺⁴ is T, G or A, and wherein the motif is within the first 35N′ terminal residues of the amino acid sequence of the polypeptide. In some embodiments, the TAT signal peptide comprises a sequence motif (X⁻¹)RR(X⁺²)(X⁺³)(X⁺⁴) that is within the first 35N′ terminal residues, wherein when X⁻¹ is H then X⁺⁴ is A. In other embodiments, the TAT signal peptide comprises a sequence motif (X⁻¹)RR(X⁺²)(X⁺³)(X⁺⁴) that is within the first 35N′ terminal residues, wherein when X⁻¹ is L then X⁺⁴ is G.

In a further aspect, the invention is directed to an isolated TAT signal peptide comprising the sequence motif (X⁻¹)RR(X⁺²)(X⁺³)(X⁺⁴), wherein RR represents two adjacent arginine residue and X designates positions restrict to other selected amino acids: X⁻¹ is M, H, A, P, K, R, N, T, G, S, D, Q, or E; X⁺² is a polar amino acid residue; and X⁺³ and X⁺⁴ are non-polar amino acid residues, and wherein the motif is not within the first 35N′ terminal residues of the amino acid sequence of the polypeptide. In some embodiments, the TAT signal polypeptide is a variant of the polypeptide having the TAT motif that is not within the first 35N′ terminal residues of the amino acid sequence of the signal peptide.

Other embodiments of the invention comprise TAT signal peptides comprising the amino acid sequences of TAT signal peptides of proteins SCO2286 (SEQ ID NO: 218), SCO3790long (SEQ ID NO: 227), and SCO6580long (SEQ ID NO: 241), SCO1590 (SEQ ID NO: 211), SCO1824 (SEQ ID NO: 213), SCO6580short (SEQ ID NO: 182), or SCO3790short (SEQ ID NO: 122).

In another aspect, the invention is directed to an isolated polynucleotide sequence comprising a polynucleotide sequence encoding a TAT signal peptide of the invention.

In some embodiments, the isolated polynucleotide sequence is a nucleic acid molecule comprising a first nucleotide sequence encoding a TAT signal sequence encompassed by the invention operably linked to a second nucleotide sequence encoding a heterologous polypeptide. In other embodiments, the invention is directed to a nucleic acid molecule comprising a first nucleotide sequence encoding a TAT signal sequence encompassed by the invention operably linked to a second nucleotide sequence encoding a homologous polypeptide.

In a further aspect, the invention is directed to an expression vector comprising a first nucleotide sequence encoding a TAT signal sequence encompassed by the invention operably linked to a second nucleotide sequence encoding a heterologous polypeptide. In one embodiment of this aspect, the invention is directed to a bacterial host cell host cell that is genetically transformed with the recombinant expression vector encompassed by the invention.

In another aspect, the invention is directed to a fusion polypeptide comprising a TAT secretion signal peptide encompassed by the invention and a heterologous polypeptide. In an embodiment of this aspect, the fusion polypeptide comprises a TAT signal peptide that is naturally expressed in Streptomyces. In another embodiment of this aspect, the heterologous polypeptide is an enzyme, growth factor or hormone. In yet another embodiment, the enzyme may be a protease, a carbohydrase, such as amylases, cellulases, xylanases, and lipases; an isomerase, such as racemases, epimerases, tautomerases, or mutases; a transferase; a glucoamylase; a kinase, an amidase, an esterase, or an oxidase. In other embodiments of this aspect, the heterologous polypeptide is not naturally associated with any secretion signal peptide.

In yet another aspect, the invention is directed to a method for producing a heterologous polypeptide comprising culturing host cells in culture medium under conditions suitable for producing said polypeptide, said host cells comprising an expression vector comprising a first nucleotide sequence encoding a TAT signal peptide encompassed by the invention operatively linked to a second nucleotide sequence encoding a heterologous polypeptide, and producing said heterologous polypeptide. In some embodiments, the method uses a TAT signal peptide of the invention that comprises the sequence motif (X⁻¹)RR(X⁺²)(X⁺³)(X⁺⁴), wherein R is arginine, X⁻¹ is amino acid M, H, A, P, K, R, N, T, G, S, D, Q or E; X⁺² is amino acid A, P, K, R, N, T, G, S, D, Q or E; X⁺³ is I, W, F, L, V, Y, M, C, H, A, P, N or T; and X⁺⁴ is Q, I, L, V, M or F, and wherein said motif is not within the first 35N′ terminal residues of the amino acid sequence of the polypeptide.

In other embodiments, the heterologous polypeptide that is produced by the method of the invention includes a TAT signal peptide that comprises the sequence motif (X⁻¹)RR(X⁺²)(X⁺³)(X⁺⁴), wherein R is arginine, X⁻¹ is amino acid H, A, P, K, R, N, T, G, S, D, Q E or L; X⁺² is A, P, K, R, N, T, G, S, D, Q or E; X⁺³ is I, W, F, L, V, Y, M, C, H, A, P, N or T; and X⁺⁴ is T, G or A, and wherein the motif is within the first 35N′ terminal residues of the amino acid sequence of the polypeptide. In some embodiments of this aspect, the step of producing the heterologous polypeptide comprises recovering the polypeptide from the culture medium.

In other embodiments of this aspect, the host cell is a prokaryotic cell. In other embodiments, the host cell is a Streptomyces bacterial cell. In yet other embodiments, the host cell is a S. coelicolor or an S. lividans bacterial cell. In further embodiments of this aspect, the method of the invention produces a heterologous polypeptide that can be an enzyme a growth factor or a hormone.

In yet another aspect, the invention is directed to a method for secreting a heterologous protein from a host microorganism comprising operably ligating a nucleotide acid sequence encoding the heterologous protein to a TAT signal sequence encompassed by the invention and inserting the ligated pair into an expression vector in a host microorganism, expressing the heterologous protein under the control of the TAT signal and secreting the heterologous protein from the microorganism by the TAT expression pathway. In some embodiments of this aspect, the expressed heterologous protein is secreted in its correctly-folded active form. In some embodiments of this aspect, the host organism is a Streptomyces strain, for example a S. lividans strain.

In another aspect, the invention provides for a method of identifying TAT signal peptides of polypeptides secreted in a microorganism comprising identifying a TAT signal peptide of a secreted polypeptide and validating the ability of said signal peptide to secrete a biological functional polypeptide. In some aspects, testing the validity of the TAT signal peptide comprises expressing a fusion polypeptide in a host microorganism, comprising the TAT signal sequence operably linked to a heterologous polypeptide and testing the biological activity of the expressed heterologous polypeptide. In some embodiments, the heterologous polypeptide is agarase.

Additional aspects and embodiments of the invention are set forth in the detailed discussion, examples and figures which follow, which are intended for illustrative purposes only and are not intended in any way to limit the invention.

### BRIEF DESCRIPTION OF THE DRAWINGS

FIG. 1A-B. The tatC mutant of S. coelicolor has pleiotropic phenotypes. Colonies of the S. coelicolor wildtype (left hand plate) and AtatC strain TP4 (right hand plate) cultured on either MS (A), or R5 (B), media.

FIG. 2 A-B illustrates a 2-dimensional gel analysis of extracellular protein fractions isolated from the S. coelicolor M145 wild type (A) and a ΔtatC mutant derivative (B). Strains were cultured on R5, and proteins associated with the cell wall were separated in the first dimension by isoelectric focusing (pH gradient 4-7) and in the second dimension by SDS PAGE. Protein spots that are circled represent proteins that migrated at specific positions in the extracellular fractions from the wild type strain but were absent from the corresponding position in extracellular fractions of the ΔtatC strain. These proteins spots were identified by in gel tryptic digest followed by mass spectrometry and identities of the proteins are indicated by the SCO number.

FIG. 3 A-D illustrates a 2-dimensional gel analysis of extracellular fractions isolated from strains grown on Complete medium (CM) (A and B) and Mannitol Soya (MS) (C and D). Proteins from the wild type S. coelicolor M145 strain are shown in A and C, and proteins from the tatC mutant strains are shown in panels B and D. Protein spots that are circled represent proteins that migrated at specific positions in the extracellular fractions from the wild type strain but were absent from the corresponding position in extracellular fractions of the ΔtatC strain. These proteins spots were identified by in gel tryptic digest followed by mass spectrometry and identities of the proteins are indicated by the SCO number.

FIG. 4 A-C Agarase is a Tat substrate. In A) the agarase signal peptide with the consecutive arginine residues of the twin-arginine motif are highlighted in bold and underlined. In B) S. coelicolor strain M145 (WT) and TP1 (ΔtatC::Apra^{R}) were grown on MM-C minimal medium for 5 days and stained with Lugol solution. In C) strains M145 (Wt) and TP4 (ΔtatC) harboring either pIJ6902 or pIJ6902-dagA in single copy were grown on MM containing glucose, apramycin and thiostrepton for 72 hours and prior to staining with Lugol solution.

FIG. 5 illustrates the export of agarase activity mediated by S. coelicolor signal peptides some of which are encompassed by the instant invention. The signal peptide is fused to the mature sequence of agarase. The y-axis shows the signal peptides from a range of cell wall associated S. coelicolor proteins (listed in Tables 3 and 4) gives the % agarase activity of the secreted protein for each fusion protein when compared with agarase including it native signal peptide (set at 100%). Each construct carries the same native agarase promoter and ribosome-binding site and therefore, the activity is a measure of the efficiency of export directed by each particular signal peptide. The assay was verified using negative controls, signal peptides that do not posses twin-arginines in the signal peptide and were strongly detected in the extracellular fractions of both the wild type (M145) and ΔtatC mutant strain by MudPIT analysis. None of these signal peptides mediated extracellular agarase activity. The signal peptides from the following proteins were also tested and were found to be negative in this assay: SCO0432, SCO0474, SCO0494, SCO0930, SCP1230, SCO1396, SCO1824, ACO1948, ACO1968, SCO2226, SCO2383, SCO2446, SCO2591, SCO2637, SCO2786, SAC02821, SCO3456, SCO4010, SCO4142, SCO4152, SCO4884, SCO4885, SCO5074, SCO5113, SCO5447, SCO5461, SCO6009, SCO6644, SCO6738, and SCO7399. The symbol * annotates that two versions of the annotated signal peptides (designated long and short), representing two alternative start sites, were tested (see Tables 3 and 4).

FIG. 6 illustrates the Tat-dependent export of agarase mediated by S. coelicolor signal peptides. For each plate, the signal peptide of each designated SCO protein, fused in frame with the mature sequence of agarase, was expressed in two S. lividans tat+ strains (1326, lower left on each plate, or 10-164, lower right of each plate), or in the 10-164 isogenic tatC strain (top of each plate). Strains were cultured on minimal medium containing 0.5% glucose for 5 days and stained with lugol solution. (Top Left) plate (DagA) corresponds to agarase expressed from an identical construct with its native signal peptide. Note that although expression of agarase in S. coelicolor is highly inhibited by glucose, heterologous expression of agarase in S. lividans, which is dagA−, is not (Servin-Gonzalez L, Jensen M R, White J, Bibb M (1994) Microbiology 140:2555-2565).

### DETAILED DESCRIPTION OF THE INVENTION

The invention will now be described in detail by way of reference only using the following definitions and examples. All patents and publications, including all sequences disclosed within such patents and publications, referred to herein are expressly incorporated by reference.

Unless defined otherwise herein, all technical and scientific terms used herein have the same meaning as commonly understood by one of ordinary skill in the art to which this invention belongs. Singleton, et al., DICTIONARY OF MICROBIOLOGY AND MOLECULAR BIOLOGY, 2D ED., John Wiley and Sons, New York (1994), and Hale & Marham, THE HARPER COLLINS DICTIONARY OF BIOLOGY, Harper Perennial, N.Y. (1991) provide one of skill with a general dictionary of many of the terms used in this invention. Although any methods and materials similar or equivalent to those described herein can be used in the practice or testing of the present invention, the preferred methods and materials are described. Numeric ranges are inclusive of the numbers defining the range. Unless otherwise indicated, nucleic acids are written left to right in 5′ to 3′ orientation; amino acid sequences are written left to right in amino to carboxy orientation, respectively. Practitioners are particularly directed to Sambrook et al., 1989, and Ausubel F M et al., 1993, for definitions and terms of the art. It is to be understood that this invention is not limited to the particular methodology, protocols, and reagents described, as these may vary.

The headings provided herein are not limitations of the various aspects or embodiments of the invention which can be had by reference to the specification as a whole. Accordingly, the terms defined immediately below are more fully defined by reference to the specification as a whole.

The term “polypeptide” as used herein refers to a compound made up of a single chain of amino acid residues linked by peptide bonds. The term “protein” as used herein may be synonymous with the term “polypeptide” or may refer, in addition, to a complex of two or more polypeptides.

The term “fusion polypeptide” or “Tat fusion polypeptide” as used herein refers to a Tat signal peptide linked to the protein of interest. It is understood that a protein of interest refers to a heterologous protein that is operably linked to the Tat signal peptide of the invention.

A “signal peptide” as used herein refers to an amino-terminal extension on a protein to be secreted. Nearly all secreted proteins use an amino-terminal protein extension which plays a crucial role in the targeting to and translocation of precursor proteins across the membrane and which is proteolytically removed by a signal peptidase during or immediately following membrane transfer.

A “Tat signal peptide” refers to a N-terminally extended sequence which includes two consecutive arginine residues and which functions in the secretion of proteins in prefolded confirmation. A “Tat signal peptide” may be interchangeably referred to as “Tat peptide” or “Tat polypeptide”.

A “tagged Tat fusion polypeptide” herein refers to a fusion polypeptide, which comprises a Tat signal peptide and a heterologous peptide, to which a tag sequence can be linked and used to identify transformants and/or to facilitate the purification of recombinant Tat fusion polypeptides.

As used herein, a “protein of interest” or “polypeptide of interest” refers to the protein to be expressed and secreted by the host cell. The protein of interest may be any protein which up until now has been considered for expression in prokaryotes. The protein of interest may be either homologous or heterologous to the host. In the first case over expression should be read as expression above normal levels in said host. In the latter case basically any expression is of course over expression.

As used herein, the term “heterologous protein” refers to a protein or polypeptide that does not naturally occur in a host cell. Examples of heterologous proteins include enzymes such as hydrolases including proteases, cellulases, amylases, other carbohydrases, and lipases; isomerases such as racemases, epimerases, tautomerases, or mutases; transferases, kinases and phosphatases. The heterologous gene may encode therapeutically significant proteins or peptides, such as growth factors, cytokines, ligands, receptors and inhibitors, as well as vaccines and antibodies. The gene may encode commercially important industrial proteins or peptides, such as proteases, carbohydrases such as amylases and glucoamylases, cellulases, oxidases and lipases. The gene of interest may be a naturally occurring gene, a mutated gene or a synthetic gene.

The term “homologous protein” refers to a protein or polypeptide native or naturally occurring in a host cell. The invention includes host cells producing the homologous protein via recombinant DNA technology. The present invention encompasses a host cell having a deletion or interruption of the nucleic acid encoding the naturally occurring homologous protein, such as a protease, and having nucleic acid encoding the homologous protein re-introduced in a recombinant form. In another embodiment, the host cell produces the homologous protein.

The term “polynucleotide” or “nucleic acid molecule” includes RNA, DNA and cDNA molecules. As used herein, the term refers to a polymeric form of nucleotides of any length, either ribonucleotides or deoxyribonucleotides. This term refers only to the primary structure of the molecule and thus includes double- and single-stranded DNA and RNA. It also includes known types of modifications, for example, labels which are known in the art, methylation, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example proteins (including for e.g., nucleases, toxins, antibodies, signal peptides, poly-L-lysine, etc.), those with intercalators (e.g., acridine, psoralen, etc.), those containing chelates (e.g., metals, radioactive metals, boron, oxidative metals, etc.), those containing alkylators, those with modified linkages (e.g., alpha anomeric nucleic acids, etc.), as well as unmodified forms of the polynucleotide. Generally, nucleic acid segments provided by this invention may be assembled from fragments of the genome and short oligonucleotide linkers, or from a series of oligonucleotides, or from individual nucleotides, to provide a synthetic nucleic acid which is capable of being expressed in a recombinant transcriptional unit comprising regulatory elements derived from a microbial or viral operon, or a eukaryotic gene.

A “heterologous” nucleic acid construct or sequence has a portion of the sequence which is not native to the cell in which it is expressed. Heterologous, with respect to a control sequence refers to a control sequence (i.e. promoter or enhancer) that does not function in nature to regulate the same gene the expression of which it is currently regulating. Generally, heterologous nucleic acid sequences are not endogenous to the cell or part of the genome in which they are present, and have been added to the cell, by infection, transfection, microinjection, electroporation, or the like. A “heterologous” nucleic acid construct may contain a control sequence/DNA coding sequence combination that is the same as, or different from a control sequence/DNA coding sequence combination found in the native cell.

As used herein, the term “vector” refers to a nucleic acid construct designed for transfer between different host cells. An “expression vector” refers to a vector that has the ability to incorporate and express heterologous DNA fragments in a foreign cell. Many prokaryotic and eukaryotic expression vectors are commercially available. Selection of appropriate expression vectors is within the knowledge of those having skill in the art.

Accordingly, an “expression cassette” or “expression vector” is a nucleic acid construct generated recombinantly or synthetically, with a series of specified nucleic acid elements that permit transcription of a particular nucleic acid in a target cell. The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid sequence to be transcribed and a promoter.

As used herein, the term “plasmid” refers to a circular double-stranded (ds) DNA construct used as a cloning vector, and which forms an extrachromosomal self-replicating genetic element in many bacteria and some eukaryotes.

As used herein, the term “selectable marker-encoding nucleotide sequence” refers to a nucleotide sequence which is capable of expression in mammalian cells and where expression of the selectable marker confers to cells containing the expressed gene the ability to grow in the presence of a corresponding selective agent.

As used herein, the term “promoter” refers to a nucleic acid sequence that functions to direct transcription of a downstream gene. The promoter will generally be appropriate to the host cell in which the target gene is being expressed. The promoter together with other transcriptional and translational regulatory nucleic acid sequences (also termed “control sequences”) are necessary to express a given gene. In general, the transcriptional and translational regulatory sequences include, but are not limited to, promoter sequences, ribosomal binding sites, transcriptional start and stop sequences, translational start and stop sequences, and enhancer or activator sequences.

“Chimeric gene” or “heterologous nucleic acid construct”, as defined herein refers to a non-native gene (i.e., one that has been introduced into a host) that may be composed of parts of different genes, including regulatory elements. A chimeric gene construct for transformation of a host cell is typically composed of a transcriptional regulatory region (promoter) operably linked to a heterologous protein coding sequence, or, in a selectable marker chimeric gene, to a selectable marker gene encoding a protein conferring antibiotic resistance to transformed cells. A typical chimeric gene of the present invention, for transformation into a host cell, includes a transcriptional regulatory region that is constitutive or inducible, a signal peptide coding sequence, a protein coding sequence, and a terminator sequence. A chimeric gene construct may also include a second DNA sequence encoding a signal peptide if secretion of the target protein is desired.

A nucleic acid is “operably linked” when it is placed into a functional relationship with another nucleic acid sequence. For example, DNA encoding a secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

As used herein, the term “gene” means the segment of DNA involved in producing a polypeptide chain, that may or may not include regions preceding and following the coding region, e.g. 5′ untranslated (5′ UTR) or “leader” sequences and 3′ UTR or “trailer” sequences, as well as intervening sequences (introns) between individual coding segments (exons).

As used herein, “recombinant” includes reference to a cell or vector, that has been modified by the introduction of a heterologous nucleic acid sequence or that the cell is derived from a cell so modified. Thus, for example, recombinant cells express genes that are not found in identical form within the native (non-recombinant) form of the cell or express native genes that are otherwise abnormally expressed, under expressed or not expressed at all as a result of deliberate human intervention.

As used herein, the terms “transformed”, “stably transformed” or “transgenic” with reference to a cell means the cell has a non-native (heterologous) nucleic acid sequence integrated into its genome or as an episomal plasmid that is maintained through two or more generations.

As used herein, the term “expression” refers to the process by which a polypeptide is produced based on the nucleic acid sequence of a gene. The process includes both transcription and translation.

The term “introduced” in the context of inserting a nucleic acid sequence into a cell, means “transfection”, or “transformation” or “transduction” and includes reference to the incorporation of a nucleic acid sequence into a eukaryotic or prokaryotic cell where the nucleic acid sequence may be incorporated into the genome of the cell (for example, chromosome, plasmid, plastid, or mitochondrial DNA), converted into an autonomous replicon, or transiently expressed (for example, transfected mRNA).

The terms “isolated” or “purified” as used herein refer to a nucleic acid or polypeptide that is removed from at least one component with which it is naturally associated.

As used herein, “substantially equivalent” can refer both to nucleotide and amino acid sequences, for example a mutant sequence, that varies from a reference sequence by one or more substitutions, deletions, or additions, the net effect of which does not result in an adverse functional dissimilarity between the reference and subject sequences. Typically, such a substantially equivalent sequence varies from one of those listed herein by no more than about 20% (i.e., the number of individual residue substitutions, additions, and/or deletions in a substantially equivalent sequence, as compared to the corresponding reference sequence, divided by the total number of residues in the substantially equivalent sequence is about 0.2 or less). Such a sequence is said to have 80% sequence identity to the listed sequence. In one embodiment, a substantially equivalent, e.g., mutant, sequence of the invention varies from a listed sequence by no more than 10% (90% sequence identity); in a variation of this embodiment, by no more than 5% (95% sequence identity); and in a further variation of this embodiment, by no more than 2% (98% sequence identity). Substantially equivalent, e.g., mutant, amino acid sequences according to the invention generally have at least 95% sequence identity with a listed amino acid sequence, whereas substantially equivalent nucleotide sequence of the invention can have lower percent sequence identities, taking into account, for example, the redundancy or degeneracy of the genetic code. For the purposes of the present invention, sequences having substantially equivalent biological activity and substantially equivalent expression characteristics are considered substantially equivalent. As used herein, the term “activity” or “biological activity” refers to an activity associated with a particular protein, such as enzymatic activity. Biological activity refers to any activity that would normally be attributed to that protein by one skilled in the art.

The recognized single letter codes for amino acid residues are used consistently herein wherein alanine (A); arginine (R); asparagine (N); aspartic acid (D); cysteine (C); glutamic acid (E); glutamine (Q); glycine (G); histidine (H); isoleucine (I); leucine (L); lysine (K); methionine (M); phenylalanine (F); proline (P); serine (S); threonine (T); tryptophan (W); tyrosine (Y) and valine (V).

The term “TATFIND”, and “TATFIND1.2” refer to a Tat substrate recognition program developed to detect putative Tat substrates in bacterial genomes. In general, the program is based on the position and sequences of the Tat motif (WO 03/079007). The motif as disclosed in WO 03/079007 is within the first 35 amino acids of a protein sequence, (X⁻¹)R⁰R⁺¹(X⁺²)(X⁺³)(X⁺⁴), wherein X⁻¹ is M, H, A, P, K, R, N, T, G, S, D, Q or E; R⁰R⁺¹ represent the twin-arginines; X⁺² is A, P, K, R, N, T, G, S, D, Q or E; X⁺³ is I, W, F, L, V, Y, M, C, H, A, P, N or T (positively charged residues being excluded from this position) and X⁺⁴ is Q, I, L, V, M or F.

The term “TatP” refers to a Tat substrate recognition program that recognizes the same Tat motif as TATFIND. The TatP program partially uses a neural network as well as a rule based classification used in the TATFIND program and reference is made to Bendtsen, J. D. et al., (2005), BMC Bioinformatics 6:167-175.

The present invention provides novel gram-positive microorganism secretion factors and methods that can be used in microorganisms to provide protein secretion and the production of proteins in secreted form.

### Tat Nucleic Acids and Amino Acids

The invention is based on the discovery of novel Tat signal peptides.

The invention comprises isolated Tat signal peptides that include, but are not limited to, a polypeptide comprising the amino acid sequence set forth as SEQ ID NO: 17, 20, 23 26 29 32 35, 38, 41, 44, 47, 50, 53, 56, 59, 62, 65, 68, 71, 74, 77, 80, 83, 86, 89, 92, 95, 98, 101, 104, 107, 110, 113 116, 119, 122, 125, 128 131, 134, 137, 140, 143, 146, 149, 152, 155, 158, 161, 164, 167, 170, 173, 176, 179, 182, 185, 188, 191, 194, 197, 200, 203, and 204-253. The Tat signal peptides include polypeptides comprising an amino acid sequence encoded by any one nucleotide sequence generated by amplifying a nucleic acid using polymerase chain reaction (PCR) primer pairs having SEQ ID NO: 15 and 16, 18 and 19, 21 and 22, 24 and 25, 27 and 28, 30 and 31, 33 and 34, 36 and 37, 39 and 40, 42 and 43, 45 and 46, 48 and 49, 51 and 52, 54 and 55, 57 and 58, 60 and 61, 63 and 64, 66 and 67, 69 and 70, 72 and 73, 75 and 76, 78 and 79, 81 and 82, 84 and 85, 87 and 88, 90 and 91, 93 and 94, 96 and 97, 99 and 100, 102 and 103, 105 and 106, 108 and 109, 111 and 112, 114 and 115, 117 and 118, 120 and 121, 123 and 124, 126 and 127, 129 and 130, 132 and 133, 135 and 136, 138 and 139, 141 and 142, 144 and 145, 147 and 148, 150 and 151, 153 and 154, 156 and 157, 159 and 160, 162 and 163, 165 and 166, 168 and 169, 171 and 172, 174 and 175, 177 and 178, 180 and 181, 183 and 184, 186 and 187, 189 and 190, 192 and 193, 195 and 196, 198 and 199, and 201 and 202.

The present invention is directed to certain novel Tat signal peptides that comprise sequences which include the motif (X⁻¹)RR(X⁺²)(X⁺³)(X⁺⁴), wherein RR represents two adjacent arginine residue and X designates positions restrict to other selected amino acids: X⁻¹ is M, H, A, P, K, R, N, T, G, S, D, Q, or E; X⁺² is A, P, K, R, N, T, G, S, D, Q or E; X⁺³ is I, W, F, L, V, Y, M, C, H, A, P, N or T; and X⁺⁴ is Q, I, L, V, M or F and wherein the motif is not within the first 35N′ terminal residues of the amino acid sequence of the polypeptide.

Tat signal sequences of the invention include the motif (X⁻¹)RR(X⁺²)(X⁺³)(X⁺⁴), wherein RR represents two adjacent arginine residue and X designates positions restrict to other selected amino acids: X⁻¹ is M, H, A, P, K, R, N, T, G, S, D, Q, or E; X⁺² is a polar amino acid residue; and X⁺³ and X⁺⁴ are non-polar amino acid residues, and wherein the motif is not within the first 35N′ terminal residues of the amino acid sequence of the polypeptide.

In the present context, non-polar amino acids are alanine, leucine, isoleucine, valine, proline, phenylalanine, tryptophan, and methionine; and polar amino acids include glycine, serine, threonine, cysteine, tyrosine, asparagine, glutamine, arginine, lysine, histidine, aspartic acid and glutamic acid.

Some preferred Tat signal peptides encompassed by the invention include, but are not limited to the Tat signal peptides of proteins SCO2286—MTPANHQAPTSAPSPAPSQSSHAPELRAAARSLGRRRFLTVTGAAAALAFAVNLPAAGTA SAA (SEQ ID NO: 218) and SCO3790 long—MRKLLPLIGTPSGSHPGGRSAMTCRFRCGDACFHEVPNTSSNEYVGDVIAGALSRRSMM RAAAVVTVAAAGAGAVGVAGAPSAQAA (SEQ ID NO: 227).

Another Tat signal peptide comprises the Tat signal peptide of protein SCO6580 long—MTPFTDSSRTDA GTDPSADGPGESLRRALGVNRRRFLSTCTAVAAGAVAAPVFGASPALAH (SEQ ID NO: 241).

In addition, the invention contemplates isolated variants of the Tat signal peptides of the invention.

In some embodiments, the variant of a Tat signal peptide is encoded by an alternative start site that leads to the translation of a shorter Tat signal peptide. Variant Tat signal peptides include, but are not limited to the Tat signal peptide of protein SCO3790, designated SCO3790short (SEQ ID NO: 122), and the Tat signal peptide of protein SCO6580, designated SCO6580 short —(SEQ ID NO: 182).

The invention also contemplates Tat signal peptides that comprise the motif (X⁻¹)RR(X⁺²)(X⁺³)(X⁺⁴), wherein RR represents two adjacent arginine residue and X designates positions restrict to other selected amino acids: X⁻¹ is H, A, P, K, R, N, T, G, S, D, Q E or L; X⁺² is A, P, K, R, N, T, G, S, D, Q or E; X⁺³ is, W, F, L, V, Y, M C, H, A, P, N or T; and X⁺⁴ is T, G or A and wherein the motif is within the first 35N′ terminal residues of the amino acid sequence of the polypeptide. In some embodiments when X⁻¹ is S then X⁺⁴ will be T; in other embodiments when X⁻¹ is H then X⁺⁴ will be A, and in other embodiments when X⁻¹ is L then X⁺⁴ will be G. The primary amino acid sequence of some of the Tat signal peptides dictates that the amino acid residues at positions X⁺³ and X⁺⁴ are non-polar amino acid residues. Thus, the invention comprises Tat signal sequences that include the motif (X⁻¹)RR(X⁺²)(X⁺³)(X⁺⁴), wherein RR represents two adjacent arginine residue and X designates positions restrict to other selected amino acids: X⁻¹ is H, A, P, K, R, N, T, G, S, D, Q E or L; X⁺² is A, P, K, R, N, T, G, S, D, Q or E; and X⁺³ and X⁺⁴ are non-polar amino acid residues.

Preferred Tat signal peptides include, but are not limited to the Tat signal sequence of protein SCO1590—MGGVSRRAFTVAALSAFTLVPEASAA (SEQ ID NO: 211) and Tat signal sequence of protein SCO1824—MTAPLSR HRRALAIPAGLAVAASLAFLP GTPAAATPAAEAA (SEQ ID NO: 213).

The invention also provides biologically active variants of any of the amino acid sequences set forth as SEQ ID NO: 17, 20, 23 26 29 32 35, 38, 41, 44, 47, 50, 53, 56, 59, 62, 65, 68, 71, 74, 77, 80, 83, 86, 89, 92, 95, 98, 101, 104, 107, 110, 113 116, 119, 122, 125, 128 131, 134, 137, 140, 143, 146, 149, 152, 155, 158, 161, 164, 167, 170, 173, 176, 179, 182, 185, 188, 191, 194, 197, 200, 203, and 204-253, and substantial equivalents thereof. Substantial equivalent Tat signal peptides have at least about 80%, or 85%, more typically at least about 90%, 91%, 92%, 93%, or 94% and even more typically at least about 95%, 96%, 97%, 98% or 99% amino acid identity, and that retain biological activity. The biological activity of a signal peptide refers to the ability of the signal peptide to translocate the polypeptide to the extracellular space. Fragments of the Tat peptides of the present invention which are capable of exhibiting biological activity are also encompassed by the present invention. The term “variant” when made in reference to a polypeptide refers to any polypeptide differing from naturally occurring polypeptides by amino acid insertions, deletions, and substitutions, or combinations thereof, which can be created using, e.g., recombinant DNA techniques. A variant of a polypeptide can also refer to a shortened version of a polypeptide that is generated by use of an alternative start codon as a translation initiation site. The alternative codon can be an in-frame or an out-of-frame start codon. Examples of variants of polypeptides that are translated from alternative start codons include but are not limited to the TAT signal polypeptides of SEQ ID NO: 122 and SEQ ID NO: 182.

Guidance in determining which amino acid residues may be replaced, added or deleted without abolishing activities of interest, may be found by comparing the sequence of the particular polypeptide with that of homologous peptides and minimizing the number of amino acid sequence changes made in regions of high homology (conserved regions) or by replacing amino acids with consensus sequence. Preferably, amino acid “substitutions” are the result of replacing one amino acid with another amino acid having similar structural and/or chemical properties, i.e., conservative amino acid replacements. “Conservative” amino acid substitutions may be made on the basis of similarity in polarity, charge, solubility, hydrophobicity, hydrophilicity, and/or the amphipathic nature of the residues involved. For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid. “Insertions” or “deletions” are typically in the range of about 1 to 5 amino acids. The variation allowed may be experimentally determined by systematically making insertions, deletions, or substitutions of amino acids in a polypeptide molecule using recombinant DNA techniques and assaying the resulting recombinant variants for activity.

Where alteration of function is desired, insertions, deletions or non-conservative alterations can be engineered to produce altered polypeptides. Such alterations can, for example, alter one or more of the biological functions or biochemical characteristics of the polypeptides of the invention. For example, such alterations may change polypeptide characteristics such as ligand-binding affinities, interchain affinities, or degradation/turnover rate. Further, such alterations can be selected so as to generate polypeptides that are better suited for expression, scale up and the like in the host cells chosen for expression.

The present invention also provides isolated Tat peptides encoded by the nucleic acids/polynucleotides of the present invention or by degenerate variants of the nucleic acids of the invention. By “degenerate variants” is intended nucleotide fragments that differ from a nucleic acid of the invention by nucleotide sequence but, due to the degeneracy of the genetic code, encode an identical polypeptide sequence.

The invention provides for fusion or chimeric polypeptides. As used herein, a fusion protein or fusion polypeptide comprises a Tat signal peptide operatively linked to a polypeptide/protein of interest. Within the fusion protein, the term “operatively linked” is intended to indicate that the Tat signal polypeptide and the polypeptide of interest are fused in-frame with one another. The Tat signal peptides are fused to the N-terminal end of the peptide of interest. Polypeptides of interest include homologous or heterologous polypeptides. Polypeptides of interest include full-length polypeptides that are naturally synthesized with a signal peptide, the mature form of the full-length polypeptides, and polypeptides that naturally lack a signal peptide. The fusion polypeptides thus comprise a Tat signal sequence that includes the motif (X⁻¹)RR(X⁺²)(X⁺³)(X⁺⁴), wherein RR represents two adjacent arginine residue and X designates positions restrict to other selected amino acids: X⁻¹ is M, H, A, P, K, R, N, T, G S, D, Q, or E; X⁺² is A, P, K, R, N, T, G, S, D, Q or E; X⁺³ is I, W, F, L, V, Y, M, C, H, A, P, N or T; and X⁺⁴ is Q, I, L V, M or F and wherein the motif is not within the first 35N′ terminal residues of the amino acid sequence of the polypeptide. In other embodiments Tat signal sequences comprised by the fusion polypeptide can include the motif (X⁻¹)RR(X⁺²)(X⁺³)(X⁺⁴), wherein RR represents two adjacent arginine residue and X designates positions restrict to other selected amino acids: X⁻¹ is M, H, A, P, K, R, N, T, G, S, D, Q, or E; X⁺² is a polar amino acid residue; and X⁺³ and X⁺⁴ are non-polar amino acid residues, and wherein the motif is not within the first 35N′ terminal residues of the amino acid sequence of the polypeptide.

As recited herein, in the present context, non-polar amino acids are alanine, leucine, isoleucine, valine, proline, phenylalanine, tryptophan, and methionine; and polar amino acids include glycine, serine, threonine, cysteine, tyrosine, asparagine, glutamine, arginine, lysine, histidine, aspartic acid and glutamic acid.

Some preferred fusion polypeptides comprise Tat signal peptides that include, but are not limited to the Tat signal peptides of proteins SCO2286—MTPANHQAPTSAPSPAPSQSSHAPELRAARSLGRRRFLTVTGAAAALAFAVNLPAAGTA SAA (SEQ ID NO: 218) and SCO3790 long—MRKLLPLIGTPSGSHPGGRSAMTCRFRCGDACFHEVPNTSSNEYVGDVIAGALSRRSMM RAAAVVTVAAAGAGAVGVAGAPSAQAA (SEQ ID NO: 227).

Another Tat signal peptide comprises the Tat signal peptide of protein SCO6580 long—MTPFTDSSRTDA GTDPSADGPGESLRRALGVNRRRFLSTCTAVAAGAVAAPVFGASPALAH (SEQ ID NO: 241).

In addition, the fusion polypeptides of the invention comprise variants of the novel Tat signal peptides. In some embodiments, the fusion polypeptide comprises a variant of a Tat signal peptide that is encoded by an alternative start site that leads to the translation of a shorter Tat signal peptide. Variant Tat signal peptides include, but are not limited to the Tat signal peptide of protein SCO3790, designated SCO3790short (SEQ ID NO: 122), and the Tat signal peptide of protein SCO6580, designated SCO6580 short—(SEQ ID NO: 182).

The invention also contemplates fusion polypeptides that comprise Tat signal peptides that include the motif (X⁻¹)RR(X⁺²)(X⁺³)(X⁺⁴), wherein RR represents two adjacent arginine residue and X designates positions restrict to other selected amino acids: X⁻¹ is H, A, P, K, R, N, T, G, S, D, Q E or L; X⁺² is A, P, K, R, N, T, G, S, D, Q or E; X⁺³ is I, W, F, L, V, Y, M, C, H, A, P, N or T; and X⁺⁴ is T, G or A and wherein the motif is within the first 35N′ terminal residues of the amino acid sequence of the polypeptide. In some embodiments when X⁻¹ is S then X⁺⁴ will be T; in other embodiments when X⁻¹ is H then X⁺⁴ will be A, and in other embodiments when X⁻¹ is L then X⁺⁴ will be G. The primary amino acid sequence of some of the Tat signal peptides dictates that the amino acid residues at positions X⁺³ and X⁺⁴ are non-polar amino acid residues. Thus, the invention comprises Tat signal sequences that include the motif (X⁻¹)RR(X⁺²)(X⁺³)(X⁺⁴), wherein RR represents two adjacent arginine residue and X designates positions restrict to other selected amino acids: X⁻¹ is H, A, P, K, R, N, T, G, S, D, Q E or L; X⁺² is A, P, K, R, N, T, G, S, D, Q or E; and X⁺³ and X⁺⁴ are non-polar amino acid residues.

Preferred fusion polypeptides comprise Tat signal peptides that include, but are not limited to the Tat signal sequence of protein SCO1590—MGGVSRRAFTVAALSAFTLVPEASAA (SEQ ID NO: 211) and Tat signal sequence of protein SCO1824—MTAPLSR HRRALAIPAGLAVAASLAFLP GTPAAATPAAEAA (SEQ ID NO: 213).

In some embodiments, the fusion polypeptide comprises a Tat signal peptide that is the secretory leader sequence of polypeptides that are naturally expressed by Streptomyces that is operably linked to a heterologous polypeptide or protein of interest. In some embodiments, the fusion polypeptide comprises a Tat signal peptide and a heterologous polypeptide such as an enzyme, a growth factor or a hormone. Enzymes include but are not limited to protease, a carbohydrase, such as amylases, cellulases, xylanases, and lipases; an isomerase, such as racemases, epimerases, tautomerases, or mutases; a transferase; a glucoamylase; a kinase, an amidase, an esterase, or an oxidase. Thus, the protein of interest may be an enzyme such as a carbohydrase, such as an α-amylase, an alkaline α-amylase, a α-amylase, a cellulase; a dextranase, an α-glucosidase, an α-galactosidase, a glucoamylase, a hemicellulase, a pentosanase, a xylanase, an invertase, a lactase, a naringanase, a pectinase or a pullulanase; a protease such as an acid protease, an alkali protease, bromelain, ficin, a neutral protease, papain, pepsin, a peptidase, rennet, rennin, chymosin, subtilisin, thermolysin, an aspartic proteinase, or trypsin; a lipase or esterase, such as a triglyceridase, a phospholipase, a pregastric esterase, a phosphatase, a phytase, an amidase, an iminoacylase, a glutaminase, a lysozyme, or a penicillin acylase; an isomerase such as glucose isomerase; an oxidoreductases, e.g., an amino acid oxidase, a catalase, a chloroperoxidase, a glucose oxidase, a hydroxysteroid dehydrogenase or a peroxidase; a lyase such as a acetolactate decarboxylase, an aspartic β-decarboxylase, a fumarese or a histadase; a transferase such as cyclodextrin glycosyltranferase; or a ligase, for example. In particular embodiments, the protein may be an aminopeptidase, a carboxypeptidase, a chitinase, a cutinase, a deoxyribonuclease, an α-galactosidase, a β-galactosidase, a β-glucosidase, a laccase, a mannosidase, a mutanase, a pectinolytic enzyme, a polyphenoloxidase, ribonuclease or transglutaminase, for example. The enzyme may be a wild-type enzyme or a variant of a wild-type enzyme. The enzyme may also be a hybrid enzyme, which comprises at least two fragments from different enzymes, for example a catalytic domain of one enzyme and a starch binding domain of a different enzyme or two fragments each fragment comprising part of the catalytic domain of the enzymes. In some embodiments, the fusion polypeptide of the invention comprises a Tat signal peptide, as recited herein, and an enzyme that is a protease, a carbohydrase, an isomerase, a glucoamylase, a kinase, an amidase, an esterase, or an oxidase.

In other embodiments, the fusion polypeptide comprises a Tat signal peptide and a heterologous polypeptide that is not naturally associated with a secretion signal peptide.

In other embodiment, the fusion polypeptide comprises a heterologous polypeptide that may be a therapeutic protein (i.e., a protein having a therapeutic biological activity). Examples of suitable therapeutic proteins include: erythropoietin, cytokines such as interferon-α, interferon-β, interferon-γ, interferon-o, and granulocyte-CSF, GM-CSF, coagulation factors such as factor VIII, factor IX, and human protein C, antithrombin III, thrombin, soluble IgE receptor α-chain, IgG, IgG fragments, IgG fusions, IgM, IgA, interleukins, urokinase, chymase, and urea trypsin inhibitor, IGF-binding protein, epidermal growth factor, growth hormone-releasing factor, annexin V fusion protein, angiostatin, vascular endothelial growth factor-2, myeloid progenitor inhibitory factor-1, osteoprotegerin, α-1-antitrypsin, α-feto proteins, DNase II, kringle 3 of human plasminogen, glucocerebrosidase, TNF binding protein 1, follicle stimulating hormone, cytotoxic T lymphocyte associated antigen 4-Ig, transmembrane activator and calcium modulator and cyclophilin ligand, soluble TNF receptor Fc fusion, glucagon like protein 1 and IL-2 receptor agonist. Antibody proteins, e.g., monoclonal antibodies that may be humanized, are of particular interest.

The invention encompasses the polynucleotides that encode the fusion polypeptides. Thus, in some embodiments, the invention is directed to isolated polynucleotides that comprise a polynucleotide sequence encoding a Tat signal peptide, as recited above, that is operably linked to a second nucleotide sequence encoding a heterologous polypeptide.

The Tat polynucleotides that encode the Tat polypeptides of the invention, as recited above, include the sequence information of the nucleic acid sequences generated by PCR using the primer pairs having SEQ ID NO: 15 and 16, 18 and 19, 21 and 22, 24 and 25, 27 and 28, 30 and 31, 33 and 34, 36 and 37, 39 and 40, 42 and 43, 45 and 46, 48 and 49, 51 and 52, 54 and 55, 57 and 58, 60 and 61, 63 and 64, 66 and 67, 69 and 70, 72 and 73, 75 and 76, 78 and 79, 81 and 82, 84 and 85, 87 and 88, 90 and 91, 93 and 94, 96 and 97, 99 and 100, 102 and 103, 105 and 106, 108 and 109, 111 and 112, 114 and 115, 117 and 118, 120 and 121, 123 and 124, 126 and 127, 129 and 130, 132 and 133, 135 and 136, 138 and 139, 141 and 142, 144 and 145, 147 and 148, 150 and 151, 153 and 154, 156 and 157, 159 and 160, 162 and 163, 165 and 166, 168 and 169, 171 and 172, 174 and 175, 177 and 178, 180 and 181, 183 and 184, 186 and 187, 189 and 190, 192 and 193, 195 and 196, 198 and 199, and 201 and 202. The primer pairs are given in Table 2. The invention also encompasses polynucleotides that are generated by degenerate variants of the primer pairs recited herein.

The polynucleotides of the present invention also include, but are not limited to a polynucleotide comprising the protein coding sequence of the Tat signal peptides having SEQ ID NO: 254-312. The polynucleotides of the present invention also include polynucleotides that hybridize under stringent conditions to the complement of any of the polynucleotides of the invention, a polynucleotide encoding any one of the Tat signal peptides of SEQ ID NO: 17, 20, 23 26 29 32 35, 38, 41, 44, 47, 50, 53, 56, 59, 62, 65, 68, 71, 74, 77, 80, 83, 86, 89, 92, 95, 98, 101, 104, 107, 110, 113 116, 119, 122, 125, 128 131, 134, 137, 140, 143, 146, 149, 152, 155, 158, 161, 164, 167, 170, 173, 176, 179, 182, 185, 188, 191, 194, 197, 200, 203, and 204-253, a polynucleotide that is an allelic variant of any polynucleotide recited above, a polynucleotide which is a species homolog of any of the polypeptides recited above, or a polynucleotide that encodes a polypeptide comprising any one of the motifs described above. An allelic variant denotes any of two or more alternative forms of a gene occupying the same chromosomal locus. Allelic variation arises naturally through mutation, and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene.

The invention also encompasses polynucleotide fragments of the nucleic acid sequences of the invention. In addition to the use of these fragments for PCR, the polynucleotide fragments can be used in various hybridization procedures or microarray procedures to identify or amplify identical or related parts of mRNA or DNA molecules from which the Tat signal peptides of the invention can be derived.

The polynucleotides of the invention additionally include the complement of any of the polynucleotides recited above.

The polynucleotides of the invention also provide polynucleotides including nucleotide sequences that are substantially equivalent to the Tat polynucleotides recited above. Polynucleotides according to the invention can have, e.g., at least about 65%, at least about 70%, at least about 75%, at least about 80%, more typically at least about 90%, and even more typically at least about 95%, sequence identity to a polynucleotide recited above. The invention also provides the complement of such polynucleotides. The polynucleotide can be DNA (genomic, cDNA, amplified, or synthetic) or RNA. Methods and algorithms for obtaining such polynucleotides are well known to those of skill in the art and can include, for example, methods for determining hybridization conditions which can routinely isolate polynucleotides of the desired sequence identities.

A Tat fusion polypeptide of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, e.g., Ausubel, et al. (eds.) CURRENT PROTOCOLS IN MOLECULAR BIOLOGY, John Wiley & Sons, 1992). A Tat signal peptide-encoding nucleic acid can be cloned into such an expression vector such that the fusion moiety e.g. polypeptide of interest, is linked in-frame to the Tat signal peptide. An expression vector comprising a Tat signal peptide-encoding polynucleotide can be any vector capable of expressing the polynucleotide encoding Tat signal peptide or Tat-fusion polypeptide in a selected host organism, and the choice of vector will depend on the host cell into which the expression vector is introduced. Thus, the invention encompasses expression vectors that comprise a first nucleotide sequence encoding a Tat signal peptide, as recited herein, operably linked to a second nucleotide sequence encoding a heterologous polypeptide.

The expression vector typically includes the components of a cloning vector, such as, for example, an element that permits autonomous replication of the vector in the selected host organism and one or more phenotypically detectable markers for selection purposes. The expression vector normally comprises control nucleotide sequences encoding a promoter, operator, ribosome binding site, translation initiation signal and optionally, a repressor gene or one or more activator genes. For expression under the direction of control sequences, the nucleic acid sequence the modified enzyme is operably linked to the control sequences in proper manner with respect to expression.

Preferably, a polynucleotide in a vector is operably linked to a control sequence that is capable of providing for the expression of the coding sequence by the host cell, i.e. the vector is an expression vector. The control sequences may be modified, for example, by the addition of further transcriptional regulatory elements to make the level of transcription directed by the control sequences more responsive to transcriptional modulators. The control sequences may in particular comprise promoters.

In the vector, the nucleic acid sequence encoding for the Tat signal peptide or the Tat signal peptide fusion polypeptide is operably combined with a suitable promoter sequence. The promoter can be any DNA sequence having transcription activity in the host organism of choice and can be derived from genes that are homologous or heterologous to the host organism. Examples of suitable promoters for directing the transcription of the modified nucleotide sequence, such as modified enzyme nucleic acids, in a bacterial host include the promoter of the Streptomyces coelicolor agarase gene dagA promoters, the promoters of the Bacillus licheniformis .alpha.-amylase gene (amyL), the aprE promoter of Bacillus subtilis, the promoters of the Bacillus stearothermophilus maltogenic amylase gene (amyM, the promoters of the Bacillus amyloliquefaciens .alpha.-amylase gene (amyQ), the promoters of the Bacillus subtilis xylA and xylB genes and a promoter derived from a Lactococcus sp.-derived promoter including the P170 promoter.

The Tat fusion polypeptide may, in addition, can comprise a tag sequence that is fused to the C-terminus of the Tat fusion polypeptide to generate a tagged Tat fusion polypeptide. Such tag sequences can be used to identify transformants and/or to facilitate the purification of recombinant Tat fusion polypeptides. For example, the Tat fusion polypeptide it may be expressed to contain a tag such as those of maltose binding protein (MBP), glutathione-S-transferase (GST) or thioredoxin (TRX), or as a His tag. Kits for expression and purification of such fusion proteins are commercially available from New England BioLab (Beverly, Mass.), Pharmacia (Piscataway, N.J.) and Invitrogen, respectively. The Tat fusion polypeptide can also be tagged with an epitope and subsequently purified by using a specific antibody directed to such epitope. One such epitope (“FLAG®) is commercially available from Kodak (New Haven, Conn.). Another tag that can be used in the invention is the c-myc tag, as is described in the examples.

This invention further provides expression vectors comprising at least a fragment of the polynucleotides set forth above and host cells or organisms transformed with these expression vectors. Useful vectors include plasmids, cosmids, lambda phage derivatives, phagemids, and the like, that are well known in the art. Accordingly, the invention also provides a vector including a polynucleotide of the invention and a host cell containing the polynucleotide. In general, the vector contains an origin of replication functional in at least one organism, convenient restriction endonuclease sites, and a selectable marker for the host cell.

The present invention further includes novel expression vectors comprising promoter elements operatively linked to polynucleotide sequences encoding a Tat fusion protein/polypeptide that comprises a Tat signal peptide of the invention and a protein of interest. The invention encompasses plasmids pTDW92, pTDW73, pTDW121, pTDW102, pTDW119, pTDW74, pTDW75, pTDW48, pTDW76, pTDW103, pTDW118, pTDW77, pTDW49, pTDW51, pTDW52, pTDW78, pTDW91, pTDW79, pTDW104, pTDW90, pTDW89, pTDW88, pTDW80, pTDW53, pTDW106, pTDW81, pTDW82, pTDW83, pTDW84, pTDW61, pTDW56, pTDW62, pTDW47, pTDW63, pTDW93, pTDW107, pTDW60, pTDW64, pTDW72, pTDW87, pTDW86, pTDW108, pTDW50, pTDW109, pTDW67, pTDW57, pTDW65, pTDW66, pTDW54, pTDW58, pTDW85, pTDW116, pTDW68, pTDW110, pTDW111, pTDW113, pTDW94, pTDW69, pTDW70, pTDW114, pTDW71, pTDW95, and pTDW115. The construction of the various expression vectors is disclosed in the examples.

### Expression Systems

In some preferred embodiments, the expression host strain for heterologously expressed protein will be a Streptomyces strain. As used herein, the genus Streptomyces includes all members known to those skilled in the art, including but not limited to S. coelicolor, S. lividans. In some embodiments, the expression host strain is S. coelicolor. In other embodiments, the expression host strain is S. lividans. The genetic elements and tools required for heterologous expression of proteins is known in the art for Streptomyces, including expression vectors, promoters, as well as fermentation protocols (Glibert et al., (1995) Crit. Rev. Biotechnology 15:13-39). Other examples of suitable bacterial host organisms are gram positive bacterial species such as Bacillaceae including Bacillus subtilis, Bacillus licheniformis, Bacillus lentus, Bacillus brevis, Bacillus stearothermophilus, Bacillus alkalophilus, Bacillus amyloliquefaciens, Bacillus coagulans, Bacillus lautus, Bacillus megaterium and Bacillus thuringiensis, and other Streptomyces species such as Streptomyces murinus, S. rubiginosus, S. griseus, S. avermitilis, lactic acid bacterial species including Lactococcus spp. such as Lactococcus lactis, Lactobacillus spp. including Lactobacillus reuteri, Leuconostoc spp., Pediococcus spp. and Streptococcus spp.

In general a DNA sequence encoding a Tat signal sequence according to the invention is linked to the 5′ end of the polynucleotide encoding the protein of interest, such that the signal sequence directs the secretion of the polypeptide sequence via the Tat pathway. Reference is made to Hopwood et al., (1985) Genetic Manipulation of Streptomyces: Laboratory Manual The John Innes Foundation, Norwich, UK, Fernandez-Abalos et al., (2003) Microbiol. 149:1623-1632; Connell, N. D. (2001) Curr. Opin. Biotechnol. 5:446-449; Binnie et al., (1997) Trends Biotechnol, 8:315-320; Van Mellaert (1993) FEMS Microbiol. Lett 114:121-128; and Baltz R., Chapter 6 “Gene Expression in Recombinant Streptomyces” in Gene Expression in Recombinant Microorganisms, Ed. Alan Smith, 1995, NY, M. Dekker).

### Cell Cultures

The present invention further provides host cells genetically transformed with the vectors of the invention, which may be, for example, an expression vector that contains the Tat signal polynucleotides and/or the Tat fusion polynucleotides of the invention. Thus, the invention encompasses a bacterial host cell transformed with an expression vector, as recited above. The vector may be, for example, in the form of a plasmid, a viral particle, a phage etc. The host cells and transformed cells can be cultured in conventional nutrient media modified as appropriate for activating promoters, selecting transformants or amplifying Tat signal and/or Tat fusion polynucleotides. The specific culture conditions, such as temperature, pH and the like will be apparent to those skilled in the art. In addition, preferred culture conditions may be found in the scientific literature such as Hopwood (2000) Practical Streptomyces Genetics, John Innes Foundation, Norwich UK; Hardwood et al., (1990) Molecular Biological Methods for Bacillus, John Wiley and from the American Type Culture Collection (ATCC). The present invention still further provides host cells genetically engineered to express the polynucleotides of the invention, wherein such polynucleotides are in operative association with a regulatory sequence heterologous to the host cell which drives expression of the polynucleotides in the cell.

The host cell can be a higher eukaryotic host cell, such as a mammalian cell, or a lower eukaryotic host cell, such as a yeast cell. Preferably, the host cell is a prokaryotic cell, such as a bacterial cell. The bacterial host cells may be from gram positive or gram negative bacteria. Preferably, the invention encompasses host cells from gram positive bacteria. A number of types of gram positive cells that may act as suitable host cells for expression of the Tat signal peptides and/or Tat fusion polypeptides include, for example, the Streptomyces, Bacillus and Lactococcus species recited herein. For industrial purposes the bacteria generally used are Bacillus subtilis, S. lividans. Preferably, the polypeptides of the invention are expressed in S. lividans cells. If the protein is made in bacteria, it may be necessary to modify the protein produced therein, for example by phosphorylation or glycosylation of the appropriate sites, in order to obtain a biologically active protein. Such covalent attachments may be accomplished using known chemical or enzymatic methods.

### Identification of Transformants

Although the presence/absence of marker gene expression suggests that a gene of interest is also present, its presence and expression should be confirmed. For example, if the nucleic acid encoding heterologous protein, such as agarase is inserted within a marker gene sequence, recombinant cells containing the insert can be identified by the absence of marker gene function. Alternatively, a marker gene can be placed in tandem with nucleic acid encoding the secretion factor under the control of a single promoter. Expression of the marker gene in response to induction or selection usually indicates expression of the secretion factor as well.

Alternatively, host cells which contain the coding sequence for a secretion factor and express the protein may be identified by a variety of procedures known to those of skill in the art. These procedures include, but are not limited to, DNA-DNA or DNA-RNA hybridization and protein bioassay or immunoassay techniques which include membrane-based, solution-based, or chip-based technologies for the detection and/or quantification of the nucleic acid or protein.

### Secretion Assays

Means for determining the levels of secretion of a heterologous or homologous protein in a gram-positive host cell and detecting secreted proteins include, using either polyclonal or monoclonal antibodies specific for the protein. Examples include enzyme-linked immunosorbent assay (ELISA), radioimmunoassay (RIA) and fluorescent activated cell sorting (FACS). These and other assays are described, among other places, in Hampton R et al (1990, Serological Methods, a Laboratory Manual, APS Press, St Paul Minn.) and Maddox D E et al (1983, J Exp Med 158:1211).

A wide variety of labels and conjugation techniques are known by those skilled in the art and can be used in various nucleic and amino acid assays. Means for producing labeled hybridization or PCR probes for detecting specific polynucleotide sequences include oligo labeling, nick translation, end-labeling or PCR amplification using a labeled nucleotide. Alternatively, the nucleotide sequence, or any portion of it, may be cloned into a vector for the production of an mRNA probe. Such vectors are known in the art, are commercially available, and may be used to synthesize RNA probes in vitro by addition of an appropriate RNA polymerase such as T7, T3 or SP6 and labeled nucleotides.

A number of companies such as Pharmacia Biotech (Piscataway N.J.), Promega (Madison Wis.), and US Biochemical Corp (Cleveland Ohio) supply commercial kits and protocols for these procedures. Suitable reporter molecules or labels include those radionuclides, enzymes, fluorescent, chemiluminescent, or chromogenic agents as well as substrates, cofactors, inhibitors, magnetic particles and the like. Patents teaching the use of such labels include U.S. Pat. Nos. 3,817,837; 3,850,752; 3,939,350; 3,996,345; 4,277,437; 4,275,149 and 4,366,241. Also, recombinant immunoglobulins may be produced as shown in U.S. Pat. No. 4,816,567 and incorporated herein by reference. An enzyme reporter assay for identifying a secreted polypeptide is described herein. While the present invention describes an agarase reporter assay for determining the presence of secreted polypeptides, it is understood that any assay that uses any one reporter described above can be used to identify the secreted polypeptides. The enzymatic activity of a secreted TAT fusion polypeptide can be determined by contacting the secreted polypeptide with a substrate and detecting the reaction product produced by the action of the enzyme on the substrate. In addition, verifying that the correct enzyme or other reporter molecule is secreted can be accomplished by performing mass spectroscopy of the secreted protein.

### Purification of Proteins

Host cells transformed with polynucleotide sequences encoding heterologous or homologous protein may be cultured under conditions suitable for the expression and recovery of the encoded protein from cell culture. “Recovering” a polypeptide from a culture medium refers to collecting the polypeptide in the culture medium into which it was secreted by the host cell. The polypeptide can also be recovered from a lysate prepared from the host cells and further purified. A secreted polypeptide may be recovered from the cell wall fraction prepared according to methods known in the art. One skilled in the art can readily follow known methods for isolating polypeptides and proteins in order to obtain one of the isolated polypeptides or proteins of the present invention. These include, but are not limited to, immunochromatography, HPLC, size-exclusion chromatography, ion-exchange chromatography, and immuno-affinity chromatography. See, e.g., Scopes, Protein Purification: Principles and Practice, Springer-Verlag (1994); Sambrook, et al., in Molecular Cloning: A Laboratory Manual; Ausubel et al., Current Protocols in Molecular Biology. The protein produced by a recombinant host cell comprising a secretion factor of the present invention will be secreted into the culture media.

The invention also encompasses fusion polypeptides that comprise a Tat signal peptide and a heterologous peptide and a polypeptide domain that will facilitate purification of soluble proteins (Kroll D J et al (1993) DNA Cell Biol 12:441-53). Such purification facilitating domains include, but are not limited to, metal chelating peptides such as histidine-tryptophan modules that allow purification on immobilized metals (Porath J (1992) Protein Expr Purif 3:263-281), protein A domains that allow purification on immobilized immunoglobulin, and the domain utilized in the FLAGS extension/affinity purification system (Immunex Corp, Seattle Wash.). The inclusion of a cleavable linker sequence such as Factor XA or enterokinase (Invitrogen, San Diego Calif.) between the purification domain and the heterologous protein can be used to facilitate purification.

In yet another aspect, the invention is directed to a method for producing a heterologous polypeptide comprising culturing host cells in culture medium under conditions suitable for producing the heterologous polypeptide, wherein the host cells contain an expression vector that comprises a first nucleotide sequence encoding a TAT signal peptide encompassed by the invention operatively linked to a second nucleotide sequence encoding a heterologous polypeptide, and producing the heterologous polypeptide. In some embodiments, the method uses a TAT signal peptide of the invention that comprises the sequence motif (X⁻¹)RR(X⁺²)(X⁺³)(X⁺⁴), wherein R is arginine, X⁻¹ is amino acid M, H, A, P, K, R, N, T, G, S, D, Q or E; X⁺² is amino acid A, P, K, R, N, T, G, S, D, Q or E; X⁺³ is I, W, F, L, V, Y, M, C, H, A, P, N or T; and X⁺⁴ is Q, I, L, V, M or F, and wherein the motif is not within the first 35N′ terminal residues of the amino acid sequence of the polypeptide.

In other embodiments, the heterologous polypeptide that is produced by the method of the invention includes a TAT signal peptide that comprises the sequence motif (X⁻¹)RR(X⁺²)(X⁺³)(X⁺⁴), wherein R is arginine, X⁻¹ is amino acid H, A, P, K, R, N, T, G, S, D, Q E or L; X⁺² is A, P, K, R, N, T, G, S, D, Q or E; X⁺³ is I, W, F, L, V, Y, M, C, H, A, P, N or T; and X⁺⁴ is T, G or A, and wherein the motif is within the first 35N′ terminal residues of the amino acid sequence of the polypeptide. In some embodiments of this aspect, the step of producing the heterologous polypeptide comprises recovering the polypeptide from the culture medium.

In other embodiments of this aspect, the host cell is a prokaryotic cell. In other embodiments, the host cell is a Streptomyces bacterial cell. In yet other embodiments, the host cell is a S. coelicolor or an S. lividans bacterial cell. In further embodiments of this aspect, the method of the invention produces a heterologous polypeptide that can be an enzyme a growth factor or a hormone.

It is contemplated that any signal sequence that directs a nascent polypeptide into a secretory pathway may be used in the present invention. It is to be understood that the any one of the newly identified signal sequences are encompassed by the invention. The signal peptides that are specifically contemplated by the instant invention included the Tat-dependent signal peptides. Specific examples include but are not limited to the Tat signal peptides listed in Tables 2, 3, and 6.

### EXPERIMENTAL

The following preparations and examples are given to enable those skilled in the art to more clearly understand and practice the present invention. They should not be considered as limiting the scope and/or spirit of the invention, but merely as being illustrative and representative thereof.

In the experimental disclosure which follows, the following abbreviations apply: eq (equivalents); M (Molar); μM (micromolar); N (Normal); mol (moles); mmol (millimoles); μmol (micromoles); nmol (nanomoles); g (grams); mg (milligrams); kg (kilograms); μg (micrograms); L (liters); ml (milliliters); μl (microliters); cm (centimeters); mm (millimeters); μm (micrometers); nm (nanometers); ° C. (degrees Centigrade); h (hours); min (minutes); sec (seconds); msec (milliseconds); TLC (thin layer achromatography); TY, tryptonlyeast extract; Ap, ampicillin; DTT, dithiotreitol; Em, erythromycin; HPDM, high phosphate defined medium; IPG, immobilized pH gradient; IPTG, isopropyl-β-D-thiogalactopyranoside; Km, kanamycin; LPDM, low phosphate defined medium; MM, minimal medium; OD, optical density; PAGE, polyacrylamide gel electrophoresis; PCR, polymerase chain reaction; Sp, spectinomycin; SSM, Schaeffer's sporulation medium; 2D, two-dimensional.

### Example 1

### Strains and Growth Conditions

Derivatives of S. coelicolor strain M145 (Bentley et al., (2002) Nature 417; 141-147; Kieser T. et al., (2000) Practical Streptomyces Genetics, The John Innes Foundation, Norfolk, UK) and S. lividans 1326 were used throughout the examples. Strains were cultured on standard laboratory growth media: minimal medium (Kieser et al., (2000) Practical Streptomyces Genetics—The John Innes Found, Norfolk, UK), complete medium (CM) Hopwood D A (1967) Bacteriol Rev 31:373-403), Mannitol soya flour (MS) (Hobbs et al., (1989) Appl Microbiol Biotechnol 31:272-277) or R5 Bierman et al (1992) Gene 116:43-49) for growth on plates and Tryptone soya broth (TSB) and Yeast extract-malt extract (YEME) for liquid growth (Hopwood D A, supra). Antibiotics were used at a final concentration of 50 μg/ml unless otherwise stated.

### Example 2

### Construction of TAT Mutants and Gross Phenotypic Analysis of ΔtatC Mutant S. coelicor Strains

To determine the impact of Tat-mediated protein export in S. coelicolor, marked mutations in each of the tatA, tatB and tatC genes and umarked in-frame deletions of tatB and tatC were constructed.

S. coelicolor tat deletion strains were prepared as follows. Antibiotic-marked and unmarked in-frame deletions were constructed using the method of Datsenko and Wanner (Datsenko et al., (2000) Proc. Natl. Acad. Sci. USA 97:6640-6645) modified as described by Gust et al., (Gust et al., (2003) Proc. Natl. Acad. Sci. USA 100:1541-1546). Cosmid 141, which carries the tatA and tatC genes, and cosmid P8, which carries the tatB gene were mutated with apramycin replacement cassettes prepared for apramycin replacement cassettes prepared for tatA, tatB, and tatC genes by using primers listed in Table 1. The mutated cosmids were transferred by mating into S. coelicolor M145, and single-crossover recombinants were selected for on MS medium containing apramycin and kanamycin (Berks et al., (2003) Adv Microbiol Physiol 47:187-254). Double crossover recombinants subsequently were selected by several rounds of growth on nonselective media followed by selection for colonies that were apramycin-resistant and kanamycin sensitive. Loss of the tat genes in each strain was confirmed by PCR, and the strains were designated TP1 (ΔtatC::ApraR), TP2 (ΔtatA::ApraR), and TP3 (ΔtatB::ApraR). Markerless strains were constructed by the method of Gust et al. (2), loss of the apramycin-resistance cassette was confirmed by PCR, and the strains were designated TP4 (ΔtatC) and TP5 (ΔtatB).

The apramycin resistance cassette from pIJ773 (Gust et al., (2003) Proc. Natl. Acad. Sci. USA 100:1541-1546) was amplified using the tatA, tatB or tatC primers, which resulted in a PCR fragments where the final 39 bp at each end corresponded to the flanking regions of the target genes. The PCR products were transformed by electroporation into strain BW25113/pIJ790 harboring either cosmid 141, which carries the tatA and tatC genes, or P8, which carries the tatB gene, and transformants were selected for apramycin resistance. The mutated cosmids were extracted, transformed into E. coli strain ETI2567 carrying helper plasmid pUZ8002 and mated into S. coelicolor M145. Single crossovers were selected by several rounds of growth on non-selective media followed by selection for colonies that were apramycin resistant and kanamycin sensitive (Gust et al. (2003) supra). The loss of the tat genes in each strain was confirmed by PCR and the strains were designated TP1 (ΔtatC::Apra^{R}), TP2 (ΔtatA::Apra^{R}) and TP3 (ΔtatB::Apra^{R}).

Marker-less strains were constructed by transforming either cosmid 141 harboring the ΔtatC::Apra^{R} allele or cosmid P8 harboring the ΔtatB::Apra^{R} allele into E. coli strain DH5α carrying plasmid pBT340, inducing expression of the FLP recombinase by growth at 42° C. and subsequent testing for colonies that were apramycin sensitive. The cosmids carrying the unmarked alleles were then transformed into E. coli strain ET12567/pUZ8002 and mated into the appropriate S. coelicolor ApraR-marked tat deletion strain with selection for kanamycin resistance. Kanamycin resistant colonies were grown for several rounds without selection and colonies that were sensitive to both apramycin and kanamycin were identified. The loss of the apramycin resistance cassette was confirmed by PCR and the strains were designated TP4 (ΔtatC) and TP5 (ΔtatB).

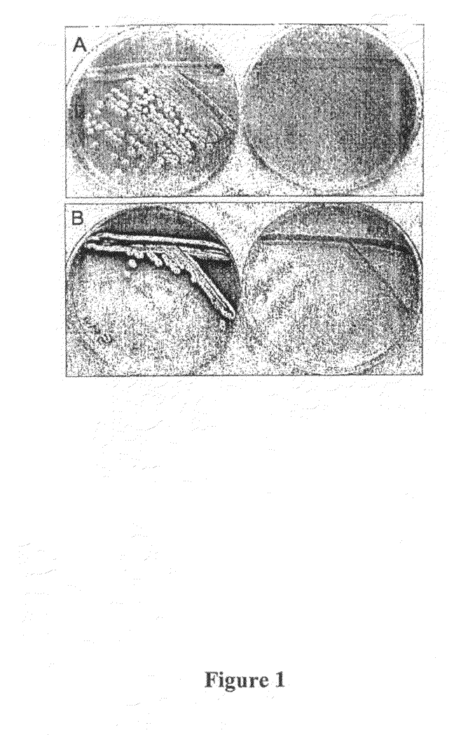

The gross phenotype of all of the coelicolor tat mutants were similar to those observed for the tatB and tatC mutants of S. lividans (Schaerlaekens et al., (2001) J Bacteriol 183:6727-6732; (Schaerlaekens et al., (2004) Microbiology 150:21-31; and FIG. 1).

When cultured on solid MS, the S. coelicolor tat mutant strains formed very small colonies that appeared to hypersporulate (FIG. 1A). Conversely, on the hyperosmotic solid R5 medium (FIG. 1B), tat mutants failed to sporulate and produced very little of the blue-pigmented antibiotic actinorhodin. In TSB liquid medium, the tat mutants grew in a very dispersed manner compared with the M145, which normally grows as pellets. Consistent with the pronounced developmental defect noted on R5 agar, tat mutant strains failed to grow in liquid YEME medium (which contains 34% sucrose) unless sucrose was excluded. S. coelicolor tat mutant mycelium were markedly more fragile than M145 mycelium and were particularly prone to lysis on shaking.

Therefore, to study the extracellular proteins of these fragile strains, it was necessary to grow the cells on solid media only and focus analysis on the exported proteins associated with the cell wall.

TABLE 1
Primers pairs used for generating tat mutants and agarase constructs

Primers for deletion of tatA

| | |
|---|---|
| TATAF | acc cac cca gcc gcc tcg gtg aga agg taa aga ctt atg att ccg ggg atc |
| | cgt cga cc |
| | (SEQ ID NO: 1) |
| TATAR | atc tcg tgc ggc ggg ccg gag atc acc ggc cct gcg tca tgt agg ctg gag |
| | ctg ctt c |
| | (SEQ ID NO: 2) |

Primers for deletion of tatB

| | |
|---|---|
| TATBF | ccg tgg tgg cgg ccc gga cat ccc aag gag ctt cag gtg att ccg ggg atc |
| | cgt cga cc |
| | (SEQ ID NO: 3) |
| TATBR | ttc ccg gcc cgc ggg ccg ggc gtg gga gga gcg ggg tca tgt agg ctg |
| | gag ctg ctt c |
| | (SEQ ID NO: 4) |

Primers for deletion of tatC

| | |
|---|---|
| TATCF | cct gcc cgc aac aag gag aag gac ccc gag ggg cgg atg att ccg ggg |
| | atc cgt cga cc |
| | (SEQ ID NO: 5) |
| TATCR | ggg cgg cga cgc gtg ccg tca ccg ccc cgg cgg tcc tca tgt agg ctg gag |
| | ctg ctt c |
| | (SEQ ID NO: 6) |

Primers for construction of agarase reporter

| | |
|---|---|
| Agarase-F | ggc ggc aag ctt aga tct acc gat tgt cac cct gcg |
| | (SEQ ID NO: 7) |
| AgaraseLead | ggc cgg gaa ttc cat atg cgt tct cct tct tcg att cc |
| | (SEQ ID NO: 8) |
| Agaraseleadb | ggc ggc gga tcc gca gac ctc gaa tgg gsa c |
| | (SEQ ID NO: 9) |
| AgaraseRxba | ggc ggc tct aga tct gtt ccg tga ggt gc |
| | (SEQ ID NO: 10) |
| AsdAFnde | ggc ggc cat atg cta ttt gcc gac tac ctt gg |
| | (SEQ ID NO: 11) |
| AadARbam | ggc ggc gga tcc ctg acg ccg ttg gat aca cc |
| | (SEQ ID NO: 12) |

Primers for construction of myc-tagged agarase

| | |
|---|---|
| kddagaf | gga att cca tat ggt caa ccg acg tga tct |
| | (SEQ ID NO: 13) |
| kddagamyc | gag gat ccc tac aga tcc tcc tcc gag atg agc ttc tgc tcc acg gcc tga |
| | tac gtc ctg a |
| | (SEQ ID NO: 14) |

### Example 3

### Construction of Reporter Plasmids

The primer sequences used to assemble a reporter construct based on the promoter and structural gene for agarase, dagA are listed in table 1. Initially a PCR product (amplified with primers Agarase-F and AgaraseL eadnde covering the dagA promoter was digested with HindIII and EcoRI and cloned into similarly digested pBluescriptII (Stratagene). A second PCR product, corresponding to the AadA streptomycin resistance gene of pIJ778 plus its promoter (amplified using primers AadAFnde and AadARbam with pIJ778 as the template), was digested with NdeI and BamHI and cloned into the above construct that had been pre-digested with the same enzymes. A third PCR fragment (amplified with primers AgaraseLEADBAM and AgaraseRxba) covering the region encoding the DagA mature protein sequence was digested with BamHI and XbaI and cloned into this to give plasmid pTDW45. Plasmid pTDW45 carries a fragment that corresponds to the dagA promoter and region of dagA encoding the mature protein sequence separated by aadA gene and flanked by BglII sites. The BglII fragment from pTDW45 was cloned into pSET152 (Bierman et al., (1992) Gene 116:43-49) that has been previously digested with BamHI to give pTDW46. This shuttle vector can replicate in E. coli and be mated into S. coelicolor where it integrates into the chromosome site-specifically. The aadA gene can be removed from this plasmid by digestion with NdeI and BamHI and replaced with DNA fragments encoding different signal peptides. A list of oligonucleotide primers and the signal peptides they amplify and the subsequent derivatives of pTDW46 carrying these signal peptides are listed in Table 2.

Plasmid pIJ6902dagA carries a C-terminally myc eptitope-tagged derivative of agarase under control of the thiostrepton-inducible promoter PtipA. The Agarase gene was amplified using primers kddagaf and kddagamyc (Table 1) digested with NdeI and BamHI and cloned into similarly digested pIJ6902 (Huang et al., (2005) Mol Microbiol 58:1276-1287). The pIJ6902dagA (PtipA-dagAmyc allele) was transferred to the φC31 site on the chromosome of M145 and TP4 as described in Bierman et al., supra.

TABLE 2

Primer pairs for amplifying Tat signal peptides

| | | | |
|---|---|---|---|
| Gene* | Oligonucleotides | Signal peptide** | Plasmid*** |

| | | | |
|---|---|---|---|
| SCO0432 | F = ggc gcg cat atg aga cgg atc cgc | MRRIRLLAAISAGLALAAGA↓ | pTDW92 |
| | ctg ttg | VAPVPALAST | |
| | (SEQ ID NO: 15) | (SEQ ID NO: 17) | |
| | R = ggc gcg gga tcc ggt gct cgc gag | | |
| | tgc ggg tac | | |
| | (SEQ ID NO: 16) | | |
| SCO0474 | F = ggc gcg cat atg gaa ccg acc gtc | MEPTVRTHPTRRLPTALVLT | pTDW73 |
| | cgc ac | AALLATGCSEQS↓DDGREPA | |
| | (SEQ ID NO: 18) | ATE | |
| | R = ggc gcg gga tcc ctc ggt cgc cgc | (SEQ ID NO: 20) | |
| | ggg ttc | | |
| | (SEQ ID NO: 19) | | |
| SCO0494 | F = ggc cgc cat atg ctc ctc aga aca | MLLRTTRTKPWRRLAAALSA | pTDW121 |
| | acg | AALGVGLLAGCGSDS↓DDP | |
| | (SEQ ID NO: 21) | ADEAGGGTPAAAGAFPVTV | |
| | R = ggc gcg gga tcc cac ggt gac cgg | (SEQ ID NO: 23) | |
| | gaa ggc | | |
| | (SEQ ID NO: 22) | | |
| SCO0736 | F = ggc cgc cat atg ggg gac ata cgc | MGDIRRRGAVALGVTALVAP | pTDW102 |
| | aga c | LTLALTA↓APAQAASC | |
| | (SEQ ID NO: 24) | (SEQ ID NO: 26) | |
| | R = ggc cgc gga tcc gca gct cgc ggc | | |
| | ctg ggc | | |
| | (SEQ ID NO: 25) | | |
| SCO0930 | F = ggc gcg cat atg aag acc tcc tgg | MKTSWRSASLVAGAAALLAL | pTDW119 |
| | cgg agc | TTACG↓QDGGGPTGSQNVG | |
| | (SEQ ID NO: 27) | ATAAPG | |
| | R = ggc gcg gga tcc gcc ggg ggc ggc | (SEQ ID NO: 29) | |
| | cgt ggc | | |
| | (SEQ ID NO: 28) | | |
| SCO1172 | F = ggc gcg cat atg gaa agg gag agg | MERERTAPVPTRRRLLKGA | pTDW74 |
| | aca gc | ALATVPYTLLSGTRAAA↓QV | |
| | (SEQ ID NO: 30) | RAVDY | |
| | R = ggc gcg gga tcc gta gtc gac ggc | (SEQ ID NO: 32) | |
| | acg gac | | |
| | (SEQ ID NO: 31) | | |
| SCO1196 | F = ggc gcg cat atg gcc ctc acc acc | MALTTRRRALTTLGAALTGA | pTDW75 |
| | cga cg | VALPA↓GTALASQ | |
| | (SEQ ID NO: 33) | (SEQ ID NO: 35) | |
| | R = ggc gcg gga tcc ctg gga cgc cag | | |
| | cgc ggt acc | | |
| | (SEQ ID NO: 34) | | |
| SCO1230 | F = ggc cgg cat atg agg aag agc agc | MRKSSIRRRATAFGTAGALV | pTDW48 |
| | ata cgg cgg agg gcg acc gcc ttc ggc | TATLIAGA↓VSAPAASA | |
| | acg gcc gga gca ctg gtc acc g | (SEQ ID NO: 38) | |
| | (SEQ ID NO: 36) | | |
| | R = ggc cgg gga tcc ggc gct cgc ggc | | |
| | ggg tgc cga gac ggc gcc ggc gat | | |
| | cag cgt ggc ggt gac cag tgc tcc ggc | | |
| | c | | |
| | (SEQ ID NO: 37) | | |
| SCO1356 | F = ggc gcg cat atg acc agc gca ccg | MTSAPFHPAAGPARRTVVA | pTDW76 |
| | ttt c | AAGAAGLTAVLAACS↓DSDD | |
| | (SEQ ID NO: 39) | GASGDGG | |
| | R = ggc gcg gga tcc gcc gcc gtc gcc | (SEQ ID NO: 41) | |
| | gga cgc | | |
| | (SEQ ID NO: 40) | | |
| SCO1396 | F = ggc gcg cat atg aca cga ctc tcc | MTRLSAALRGLATTFAALLA | pTDW103 |
| | gcc | VTAVPAAS↓APATAQARHE | |
| | (SEQ ID NO: 42) | (SEQ ID NO: 44) | |
| | R = ggc gcg gga tcc ctc gtg ccg tgc | | |
| | ctg cgc | | |
| | (SEQ ID NO: 43) | | |
| SCO1432 | F = ggc gcg cat atg caa cga cgg acc | MQRRTRPTRTPGGLPLMSR | pTDW118 |
| | cgc ccg acc cgg aca | VEQPSRRALLAAAVAAAAVT | |
| | (SEQ ID NO: 45) | GGA↓IPATARAA | |
| | R = ggc gcg aga tct tgc ggc ccg ggc | (SEQ ID NO: 47) | |
| | ggt cgc cgg gat c | | |
| | (SEQ ID NO: 46) | | |
| SCO1565 | F = ggc gcg cat atg gga acg cag gag | MGTQESDERAGGGTGRRA | pTDW77 |
| | tcg gac | LLGAAVLGAGGAVLGLPGTA | |
| | (SEQ ID NO: 48) | RA↓AGT | |
| | R = ggc gcg gga tcc ggt gcc ggc ggc | (SEQ ID NO: 50) | |
| | tct cgc cgt g | | |
| | (SEQ ID NO: 49) | | |
| SCO1590 | F = ggc ggc cat atg ggc ggt gtc tcg | MGGVSRRAFTVAALSAFTLV | pTDW49 |
| I | cgc cgt gcc ttc acg gtg gcg gcg ttg | PEA↓SAA | |
| | tcg gcg | (SEQ ID NO: 53) | |
| | (SEQ ID NO: 51) | | |
| | R = ggc ggc gga tcc cgc cga cgc ctc | | |
| | ggg cac gag cgt gaa cgc cga caa | | |
| | cgc cgc cac cgt | | |
| | (SEQ ID NO: 52) | | |
| SCO1590 | F = ggc ggc cat atg ggc ggt gtc tcg | MGGVSRRAFTVAALSAFTLV | pTDW51 |
| II | cgc cgt gcc ttc acg gtg gcg gcg ttg | PEASAA↓TP | |
| | tcg gcg | (SEQ ID NO: 56) | |
| | (SEQ ID NO: 54) | | |
| | R = ggc ggc gga tcc ggg ggt cgc cgc | | |
| | cga cgc ctc ggg cac gag cgt gaa | | |
| | cgc cga caa cgc cgc cac cgt | | |
| | (SEQ ID NO: 55) | | |
| SCO1639 | F = ggc gcg cat atg cgc cga cgc tca | MRRRSLLIAVPTGLVTLAAC | pTDW52 |
| | ctc ctc atc gcc gtc ccc acg gga ctg | GDSDDSGSSSNA↓SESPSP | |
| | gtc acg ctc gcc gcc tgc ggt gac agc | DASATSAA | |
| | gac gac tcc gg | (SEQ ID NO: 59) | |
| | (SEQ ID NO: 57) | | |
| | R = ggc gcg gga tcc cgc ggc cga ggt | | |
| | ggc cga cgc gtc ggg cga cgg gct ctc | | |
| | gct cgc gtt gct cga cga gcc gga gtc | | |
| | gtc gct gtc acc | | |
| | (SEQ ID NO: 58) | | |
| SCO1824 | F = ggc gcg cat atg acc gct ccc ctc | MTAPLSRHRRALAIPAGLAV | pTDW78 |
| | tcg | AASLAFLPGTPAA↓ATPAAE | |
| | (SEQ ID NO: 60) | AAP | |
| | R = ggc gcg gga tcc ggg cgc ggc ctc | (SEQ ID NO: 62) | |
| | ggc cgc | | |
| | (SEQ ID NO: 61) | | |
| SCO1906 | F = ggc gcg cat atg tcg ctc act cgc | MSLTRRDFAGRSALTGAGV | pTDW91 |
| | agg | VLAGSVGALA↓TAPNALAST | |
| | (SEQ ID NO: 63) | (SEQ ID NO: 65) | |
| | R = ggc gcg gga tcc cgt gga cgc gag | | |
| | ggc gtt g | | |
| | (SEQ ID NO: 64) | | |
| SCO1948 | F = ggc gcg cat atg aga cga aga gcg | MRRRARSILAVGALLIGGAS | pTDW79 |
| | aga tc | FA↓PIAQAQP | |
| | (SEQ ID NO: 66) | (SEQ ID NO: 68) | |
| | R = ggc gcg gga tcc ggg ttg tgc ctg | | |
| | ggc gat g | | |
| | (SEQ ID NO: 67) | | |
| SCO1968 | F = ggc gcg cat atg cac gtg cgc gca | MHVRAVAVTTTALLGVALT↓ | pTDW104 |
| | gta g | APLSHARADEA | |
| | (SEQ ID NO: 69) | (SEQ ID NO: 71) | |
| | R = ggc gcg gga tcc cgc ctc gtc cgc | | |
| | ccg cgc gtg | | |
| | (SEQ ID NO: 70) | | |
| SCO2068 | F = ggc gcg cat atg acc agt cga cac | MTSRHRASENSRTPSRRTV | pTDW90 |
| | aga gc | VKAAAAGAVLAAPLAAA↓LP | |
| | (SEQ ID NO: 72) | AGAADAAP | |
| | R = ggc gcg gga tcc ggg ggc cgc gtc | (SEQ ID NO: 74) | |
| | ggc cgc | | |
| | (SEQ ID NO: 73) | | |
| SCO2226 | F = ggc gcg cat atg cct gcc acg cgc | MPATRRTARVRRVAAVTVT | pTDW89 |
| | cgt ac | ALAAA↓LLPPLAARADT | |
| | (SEQ ID NO: 75) | (SEQ ID NO: 77) | |
| | R = ggc gcg gga tcc ggt gtc ggc ccg | | |
| | tgc ggc gag | | |
| | (SEQ ID NO: 76) | | |
| SCO2286 | F = ggc gcg cat atg aca ccc gcg aac | MTPANHQAPTSAPSPAPSQ | pTDW88 |
| | cac | SSHAPELRAAARSLGRRRFL | |
| | (SEQ ID NO: 78) | TVTGAAAALAFAVNLPA↓AG | |
| | R = ggc gcg gga tcc ctc ggc ggc gct | TASAAE | |
| | cgc ggt g | (SEQ ID NO: 80) | |
| | (SEQ ID NO: 79) | | |
| SCO2383 | F = ggc gcg cat atg cgt ctc aga cgc | MRLRRPRRPSDGPPGRSG | pTDW80 |
| | ccc cgc ag | ARRLRVASAPLIPLTVIGAVV | |
| | (SEQ ID NO: 81) | AGLIGPEPPSDG↓PLPGTSA | |
| | R = ggc gcg gga tcc gct gtc ggc gac | VADS | |
| | ggc act g | (SEQ ID NO: 83) | |
| | (SEQ ID NO: 82) | | |
| SCO2446 | F = ggc gcg cat atg agc aca gga cca | MSTGPVRRGAALLSAGLVIA | pTDW53 |
| | gtc aga cgc ggg gcg gcc ctc ctg tcg | LLPA↓GTSAAQ | |
| | gcg ggg ctc gtg | (SEQ ID NO: 86) | |
| | (SEQ ID NO: 84) | | |
| | R = ggc gcg gga tcc ctg cgc cgc cga | | |
| | ggt ccc ggc cgg cag cag cgc gat | | |
| | cac gag ccc cgc cga cag gag | | |
| | (SEQ ID NO: 85) | | |
| SCO2591 | F = ggc gcg cat atg ttc aca tca cgc | MFTSRARLRTRRSRLLTCTA | pTDW106 |
| | gcg cgc ctc aga | LAVAAGMLVTT↓PAVAAD | |
| | (SEQ ID NO: 87) | (SEQ ID NO: 89) | |
| | R = ggc gcg gga tcc gtc ggc ggc gac | | |
| | ggc ggg ag | | |
| | (SEQ ID NO: 88) | | |
| SCO2637 | F = ggc gcg cat atg acc aac agc ccc | MTNSPQREPIPGARRAARL | pTDW81 |
| | cag | ALATGLAAALA↓AVGPVPVA | |
| | (SEQ ID NO: 90) | LAAD | |
| | R = ggc gcg gga tcc gtc ggc ggc gag | (SEQ ID NO: 92) | |
| | ggc cac | | |
| | (SEQ ID NO: 91) | | |
| SCO2758 | F = ggc gcg cat atg cac cac agc agc | MHHSSTAGTGSTAQPSRRS | pTDW82 |
| | acg g | VLTATAVVTAALA↓AGGGTA | |
| | (SEQ ID NO: 93) | YADA | |
| | R = ggc gcg gga tcc ggc gtc ggc gta | (SEQ ID NO: 95) | |
| | ggc cgt ac | | |
| | (SEQ ID NO: 94) | | |
| SCO2780 | F = ggc gcg cat atg tcc cac gcc agc | MSHASATHPTRRGILAAGG | pTDW83 |
| | gct ac | ALGLGAVLAACG↓DGDGKS | |
| | (SEQ ID NO: 96 | DGA GD | |
| | R = ggc gcg gga tcc gtc gcc cgc ccc | (SEQ ID NO: 98) | |
| | atc gct c | | |
| | (SEQ ID NO: 97) | | |
| SCO2786 | F = ggc gcg cat atg aga cct cat cga | MRPHRRHHRTTPRITRLLGS | pTDW84 |
| | cgg cac | LLLVAAVGAMTTGA↓APVRK | |
| | (SEQ ID NO: 99) | AAAEP | |
| | R = ggc gcg gga tcc cgg ttc cgc ggc | (SEQ ID NO: 101) | |
| | cgc ctt c | | |
| | (SEQ ID NO: 100) | | |
| SCO2821 | F = ggc gcg cat atg aga cga cca gtc | MRRPVALRLQAALGTLALAA | pTDW61 |
| | gcg ctg | A↓AGVVLTMPEAAAAAG | |
| | (SEQ ID NO: 102) | (SEQ ID NO: 104) | |
| | R = ggc gcg gga tcc gcc ggc cgc tgc | | |
| | cgc cgc ctc | | |
| | (SEQ ID NO: 103) | | |
| SCO3053 | F = ggc gcg cat atg tcc aga ttc cgt | MSRFRSRAAVTAGLTAIAVA | pTDW56 |
| | tca cgc gcc gcg gtc acc gcg ggt ctg | AGCLVVG↓PVQAAT | |
| | acg gcc atc gcg gtg gcc | (SEQ ID NO: 107) | |
| | (SEQ ID NO: 105) | | |
| | R = ggc gcg gga tcc ggt ggc ggc ctg | | |
| | gac ggg tcc gac gac gag gca gcc | | |
| | ggc ggc cac cgc gat ggc cgt cag | | |
| | (SEQ ID NO: 106) | | |
| SCO3456 | F = ggc gcg cat atg cgg cac agc gga | MRHSGRRVKRATVLAAGAA | pTDW62 |
| | cgg | VAGLLTAA↓CSSPDTTGVE | |
| | (SEQ ID NO: 108) | (SEQ ID NO: 110) | |
| | R = ggc gcg gga tcc ctc gac ccc cgt | | |
| | ggt atc | | |
| | (SEQ ID NO: 109) | | |
| SCO3471 | F = ggc cgg cat atg gtc aac cga cgt | MVNRRDLIKWSAVALGAGA | pTDW47 |
| (dagA) | gat ctc atc aag tgg agt gcc gtc gca | GLAGPAPAAHA↓ADL | |
| | ctc gga gcg ggt g | (SEQ ID NO: 113) | |
| | (SEQ ID NO: 111) | | |
| | R = ggc cgg gga tcc gag gtc tgc ggc | | |
| | atg agc ggc ggg tgc ggg acc cgc | | |
| | gag ccc cgc acc cgc tcc gag tgc gac | | |
| | g | | |
| | (SEQ ID NO: 112) | | |
| SCO3484 | F = ggc gcg cat atg acc aag cct gtt | MTKPVVPSGVSRRGFLGGS | pTDW63 |
| | gtg | LGVAGAVLLAA↓CSGGGNS | |
| | (SEQ ID NO: 114) | SQGSG | |
| | R = ggc gcg gga tcc acc gga gcc ttg | (SEQ ID NO: 116) | |
| | gga cga g | | |
| | (SEQ ID NO: 115) | | |
| SCO3790 | F = ggc gcg cat atg cgc aag ctc ctg | MRKLLPLIGTPSGSHPGGRS | pTDW93 |
| long | ccg ttg | AMTCRFRCGDACFHEVPNT | |
| | (SEQ ID NO: 117) | SSNEYVGDVIAGALSRRSM | |
| | R = ggc gcg gga tcc ggg cgc ggc ctg | MRAAAVVTVAAAGAGAVGV | |
| | cgc cga c | AG↓APSAQAAP | |
| | (SEQ ID NO: 118) | (SEQ ID NO: 119) | |
| SCO3790 | F = ggc gcg cat atg ccg aac acc agc | MPNTSSNEYVGDVIAGALSR | pTDW107 |
| short | tcc aac g | RSMMRAAAVVTVAAAGAGA | |
| | (SEQ ID NO: 120) | VGVAG↓APSAQAAP | |
| | R = ggc gcg gga tcc ggg cgc ggc ctg | (SEQ ID NO: 122) | |
| | cgc cga c | | |
| | (SEQ ID NO: 121) | | |
| SCO4142 | F = ggc gcg cat atg aac cgg cgg gcc | MNRRALALGALAVSGALALT | pTDW60 |
| | ctc gct c | ACG↓SDDTGGNSGSDSSSA | |
| | (SEQ ID NO: 123) | AANS | |
| | R = ggc gcg gga tcc gct gtt ggc ggc | (SEQ ID NO: 125) | |
| | ggc gga g | | |
| | (SEQ ID NO: 124) | | |
| SCO4152 | F = ggc gcg cat atg cca gtc aca gca | MPVTAQPHQPRRRRRTSRL | pTDW64 |
| | cag | LVVAATVVTAGVLAAAALPA | |
| | (SEQ ID NO: 126) | ↓SASAGG | |
| | R = ggc gcg gga tcc ccc gcc cgc gct | (SEQ ID NO: 128) | |
| | cgc cga g | | |
| | (SEQ ID NO: 127) | | |
| SCO4672 | F = ggc gcg cat atg acc cac acc tca | MTHTSRRGVLAAFAASAAT | pTDW72 |
| | cgg | VPFGGAAAASPARATATAA | |
| | (SEQ ID NO: 129) | SA↓VDPAAASAAP | |
| | R = ggc gcg gga tcc cgg agc ggc gga | (SEQ ID NO: 131) | |
| | cgc agc | | |
| | (SEQ ID NO: 130) | | |
| SCO4884 | F = ggc gcg cat atg cgt cgg aca tca | MRRTSRLIRVAVGVASLALA | pTDW87 |
| | aga c | ATA↓CGGTSGESGDD | |
| | (SEQ ID NO: 132) | (SEQ ID NO: 134) | |
| | R = ggc gcg gga tcc gtc gtc gcc gct | | |
| | ctc gcc gct g | | |
| | (SEQ ID NO: 133) | | |
| SCO4885 | F = ggc gcg cat atg cgc cgg att tcc | MRRISRITVAGAATASLALAL | pTDW86 |
| | cgg atc | AACG↓GTSTDSGSES | |
| | (SEQ ID NO: 135) | (SEQ ID NO: 137) | |
| | R = ggc gcg gga tcc gga ctc cga acc | | |
| | ggc gtc | | |
| | (SEQ ID NO: 136) | | |
| SCO5074 | F = ggc cgc cat atg acc tcg tcg ctg | MTSSLHHAIRLTTASAIALGG | pTDW108 |
| | cac | LVTLGTS↓AHAASV | |
| | (SEQ ID NO: 138) | (SEQ ID NO: 140) | |
| | R = ggc cgc gga tcc gac gct cgc ggc | | |
| | gtg cgc | | |
| | (SEQ ID NO: 139) | | |
| SCO5113 | F = ggc ggc cat atg agc att ctc cgt | MSILRNRTATAAIVAVAAGAL | pTDW50 |
| | aac cgc acc gcc acg gcc gcc ata gtc | TLTACGG↓GDSDSSA | |
| | gcg gtc gca gcc ggc gcc ctg | (SEQ ID NO: 143) | |
| | (SEQ ID NO: 141) | | |
| | R = ggc ggc gga tccggc gga gct gtc | | |
| | gct gtc gcc acc acc gca ggc ggt gag | | |
| | ggt cag ggc gcc ggc tgc gac cgc | | |
| | (SEQ ID NO: 142) | | |
| SCO5447 | F = ggc cgc cat atg ttg agc agc agc | MLSSSTPHRRTSHTTSHTS | pTDW109 |
| | act c | RRTAHRRAAAVALAGVAALI | |
| | (SEQ ID NO: 144) | ATAVQS↓GTATAAPD | |
| | R = ggc cgc gga tcc gtc ggg ggc cgc | (SEQ ID NO: 146) | |
| | ggt ggc | | |
| | (SEQ ID NO: 145) | | |
| SCO5461 | F = ggc gcg cat atg atc acc act agt | MITTSLRRRTAAAVLSLSAVL | pTDW67 |
| | ctg c | ATTAATAPGAAPAPSA↓APA | |
| | (SEQ ID NO: 147) | KAAP | |
| | R = ggc gcg gga tcc cgg ggc ggc ctt | (SEQ ID NO: 149) | |
| | ggc ggg | | |
| | (SEQ ID NO: 148) | | |
| SCO5660 | F = ggc gcg cat atg cgt gcc cgt agc | MRARSHAVLAASVVLALAA | pTDW57 |
| | cat gca gtt ctc gcc gcc tcg gtc gtc | GPLA↓APAFAAP | |
| | ctc gcc ctg gcc g | (SEQ ID NO: 152) | |
| | (SEQ ID NO: 150) | | |
| | R = ggc gcg gga tcc cgg cgc ggc gaa | | |
| | ggc ggg ggc cgc cag cgg gcc ggc | | |
| | ggc cag ggc gag gac gac c | | |
| | (SEQ ID NO: 151) | | |
| SCO6009 | F = ggc gcg cat atg aac acg cgt atg | MNTRMRRAAVAVATTAMAV | pTDW65 |
| | cgt c | SLAA↓CGSAKE | |
| | (SEQ ID NO: 153) | (SEQ ID NO: 155) | |
| | R = ggc gcg gga tcc ctc ctt ggc gct | | |
| | gcc aca g | | |
| | (SEQ ID NO: 154) | | |
| SCO6052 | F = ggc gcg cat atg tcc acg tcc cgc | MSTSRNAATRRQVLARTGA | pTDW66 |
| | aac g | LGAGIAFTGALSELFA↓GTAA | |
| | (SEQ ID NO: 156) | AQP | |
| | R = ggc gcg gga tcc ggg ctg ggc ggc | (SEQ ID NO: 158) | |
| | ggc ggt g | | |
| | (SEQ ID NO: 157) | | |
| SCO6198 | F = ggc gcg cat atg gac acc act tac | MDTTYWSRRRVLTVLGAAT | pTDW54 |
| | tgg agc aga cgt cgc gtc ctc acc gtg | AATSIPLA↓APSRALAA | |
| | ctc ggc gcc gcc acc gcg gcg ac | (SEQ ID NO: 161) | |
| | (SEQ ID NO: 159) | | |
| | R = ggc gcg gga tcc ggc ggc gag agc | | |
| | gcg gga cgg ggc ggc gag agg gat | | |
| | cga ggt cgc cgc ggt ggc ggc gcc | | |
| | (SEQ ID NO: 160) | | |
| SCO6199 | F = ggc gcg cat atg cac cgc aca aga | MHRTRPGGHPRSIRFATLAI | pTDW58 |
| | ccc ggg ggc cac ccc cga tcc ata cgc | SLTAGSALMT↓ASPAVAVT | |
| | ttc gcc aca ctc gcg atc tcc ctg a | (SEQ ID NO: 164) | |
| | (SEQ ID NO: 162) | | |
| | R = ggc gcg gga tcc ggt gac ggc gac | | |
| | ggc cgg tga cgc cgt cat gag cgc gga | | |
| | gcc cgc ggt cag gga gat cgc gag tgt | | |
| | g | | |
| | (SEQ ID NO: 163) | | |
| SCO6272 | F = ggc gcg cat atg acc gag gtg tcc | MTEVSRRKLMKGAAVSGGA | pTDW85 |
| | cgc | LALPALGAPPATA↓APAAGP | |
| | (SEQ ID NO: 165) | EDLPGPAAAAAG | |
| | R = ggc gcg gga tcc ccc tgc cgc ggc | (SEQ ID NO: 167) | |
| | cgc tgc | | |
| | (SEQ ID NO: 166) | | |
| SCO6281 | F = ggc gcg cat atg tcc gcc att tct cgc | MSAISRRRFIRHGAVAGGAA | pTDW116 |
| | (SEQ ID NO: 168) | VALPALGGWAGEAFA↓AQP | |
| | R = ggc gcg gga tcc ggc gat cgc cgc | SPAAAIA | |
| | ggc ggg tg | (SEQ ID NO: 170) | |
| | (SEQ ID NO: 169) | | |
| SCO6418 | F = ggc gcg cat atg gac aga cga gcc | MDRRALMLATGGLLGAAGA | pTDW68 |
| | ctc | AQLTAAPSAVA↓AGTDAAT | |
| | (SEQ ID NO: 171) | (SEQ ID NO: 173) | |
| | R = ggc gcg gga tcc ggt cgc ggc gtc | | |
| | cgt ccc | | |
| | (SEQ ID NO: 172) | | |
| SCO6457 | F = ggc gcg cat atg ccg cac tcg ccc | MPHSPVSPAESPAPQPGRP | pTDW110 |
| | gtg tc | RPVVSRRRLLEGGAAVLGA | |
| | (SEQ ID NO: 174) | LALSASPLTAQA↓AVRRAAA | |
| | R = ggc gcg gga tcc ctc gtc ggc cgc | DE | |
| | cgc ccg ccg tac | (SEQ ID NO: 176) | |
| | (SEQ ID NO: 175) | | |
| SCO6580 | F = ggc cgc cat atg acc ccg ttc acc | MTPFTDSSRTDAGTDPSAD | pTDW111 |
| long | gac | GPGESLRRALGVNRRRFLS | |
| | (SEQ ID NO: 177) | TCTAVAAGAVAAPVFG↓ASP | |
| | R = ggc cgc gga tcc ccg gtc gtg ggc | ALAHDR | |
| | gag cgc | (SEQ ID NO: 179) | |
| | (SEQ ID NO: 178) | | |
| SCO6580 | F = ggc gcg cat atg aac cgg cgc cgc | MNRRRFLSTCTAVAAGAVA | pTDW113 |
| short | ttc | APVFG↓ASPALAHDR | |
| | (SEQ ID NO: 180) | (SEQ ID NO: 182) | |
| | R = as above; | | |
| | (SEQ ID NO: 181) | | |
| SCO6594 | F = ggc gcg cat atg ccg tct tcc ccc | MPSSPTPSPTPSGAPEPSG | pTDW94 |
| | acg | VRRRSLLAAAAAAPVLASLA | |
| | (SEQ ID NO: 183) | GA↓GTAAADS | |
| | R = ggc gcg gga tcc gga gtc ggc ggc | (SEQ ID NO: 185) | |
| | ggc ggt tc | | |
| | (SEQ ID NO: 184) | | |
| SCO6644 | F = ggc gcg cat atg cgc tcc cac cgc | MRSHRRPRLLAPFLLVPLMA | pTDW69 |
| | cgc | GCFASGGG↓DDSASGDG | |
| | (SEQ ID NO: 186) | (SEQ ID NO: 188) | |
| | R = ggc gcg gga tcc acc gtc acc gga | | |
| | cgc gga a | | |
| | (SEQ ID NO: 187) | | |
| SCO6691 | F = ggc gcg cat atg gcc gaa gtg aac | MAEVNRRRFLQLAGATTAF | pTDW70 |
| | cgg | SALS↓ASIDRAAALP | |
| | (SEQ ID NO: 189) | (SEQ ID NO: 191) | |
| | R = ggc gcg gga tcc cgg gag tgc cgc | | |
| | ggc ccg gtc | | |
| | (SEQ ID NO: 190) | | |
| SCO6738 | F = ggc cgc cat atg aac cgc ctg cgc | MNRLRTLTATTAALAASLLV | pTDW114 |
| | acc | PSLLTA↓PAHADGS | |
| | (SEQ ID NO: 192) | (SEQ ID NO: 194) | |
| | R = ggc cgc gga tcc gga gcc gtc ggc | | |
| | gtg ggc | | |
| | (SEQ ID NO: 193) | | |
| SCO7399 | F = ggc gcg cat atg aga cgc ctc ctg | MRRLLLTAAATTAAALTLAA | pTDW71 |
| | ctc | CG↓TTEPAADK | |
| | (SEQ ID NO: 195) | (SEQ ID NO: 197) | |
| | R = ggc gcg gga tcc ctt gtc ggc ggc | | |
| | ggg ttc | | |
| | (SEQ ID NO: 196) | | |
| SCO7631 | F = ggc gcg cat atg agc aca gag gaa | MSTEEQSSGRRHPDRRVLL | pTDW95 |
| | cag | RAAVAVPPAGAAVAGTAAV | |
| | (SEQ ID NO: 198) | PA↓QAADAAG | |
| | R = ggc gcg gga tcc gcc ggc cgc gtc | (SEQ ID NO: 200) | |
| | ggc cgc ctg | | |
| | (SEQ ID NO: 199) | | |
| SCO7677 | F = ggc cgc cat atg tcc aga cag atc | MSRQIDRRSFLRRGAAGAA | pTDW115 |
| | gat c | ALAVGPGLLAA↓CSTDEPGS | |
| | (SEQ ID NO: 201) | AGNPG | |
| | R = ggc cgc gga tcc tcc ggg gtt tcc | (SEQ ID NO: 203) | |
| | ggc gga g | | |
| | (SEQ ID NO: 202) | | |

*The SCO number for each protein whose signal peptide was cloned in frame with mature agarase is given.
**The signal peptide coding sequence that was amplified for each secreted protein is shown, the arrow indicates the most likely position of signal peptide cleavage as determined by SignalP
***The plasmid designation of the final construct carrying the particular signal peptide-agarase fusion is given.

### Example 4

### Identification of Tat-Dependent Proteins by 2D-Gel Electrophoresis (2-DGE)

To identify proteins secreted via the TAT pathway, extracellular proteins derived from the S. coelicolor parent strain (M145) and the tatC mutant strain were analyzed by two-dimensional gel electrophoresis (2-DGE).

Protein were prepared for 2D gel analysis or MUDPIT by following the method of (Hesketh et al., (2002) Mol. Microbiol. 46:917-932; Yu et al., (2003) Anal. Chem. 75:6023-6028; and Perkins et al., (1999) Electrophoresis 20:3551-3567) S. coelicolor strains M145 or TP1 tatC were grown by inoculating 10⁶ spores onto sterile cellophane disks placed on the surface of complete medium (CM), R5 or mannitol soya (MS) media. After incubation for 48 hours at 30° C. the biomass was scraped from the disks, dispersed in 5M LiCl solution and left on ice for 30 minutes. The suspensions were vortexed for 2-3 minutes and the biomass removed by centrifugation (1800 g for 5 min) followed by passage through a 0.45 um filter. Protein were precipitated from the LiCl solution by addition of trichloroacetic acid to a final concentration of 20%, incubated on ice for 30 min and centrifuged at 1800 g for 15 min. After centrifugation, the mixture had settled into two phases. The upper phase was removed and discarded and water was added to the lower phase to adjust it back to the original volume. The solution turned cloudy so it was again centrifuged at full speed for 15 min, after which the precipitated protein formed a pellet. The pellet was washed three times with −20° C. acetone and then air-dried.

For 2D gel analysis, the protein samples were resuspended in IEF sample-buffer (8M urea, 0.5% CHAPS, 0.2% DTT, 0.5% IPG buffer pH 4-7 (Amersham Biosciences), 0.002% bromophenol blue) and protein concentration was determined using the Biorad DC protein assay. The proteins in the samples were separated in the first dimension by their differing isoelectric points. 500-1000 μg samples of protein were loaded onto Amersham Biosciences 18 cm Immobline Drystrip pH 4-7 or pH 6-11 iso-electric focusing gels and resolved by electrophoresis for 33 kVh using an Amersham Biosciences Ettan IPGphor iso-electric focusing unit. Focussed strips were treated as described by Hesketh et al., (2002) supra and then separated in the second dimension by SDS-PAGE using Amersham Biosciences DALT 12.5% gels. The gels were stained using a colloidal Coomassie Blue stain and scanned with a Proxpress Proteomic Imaging System scanner for later comparison. Protein spots of interest were subsequently excised from the gel, digested with trypsin and identified by MALDI-TOF peptide mass fingerprint analysis as described previously (Hesketh et al., (2002) supra).

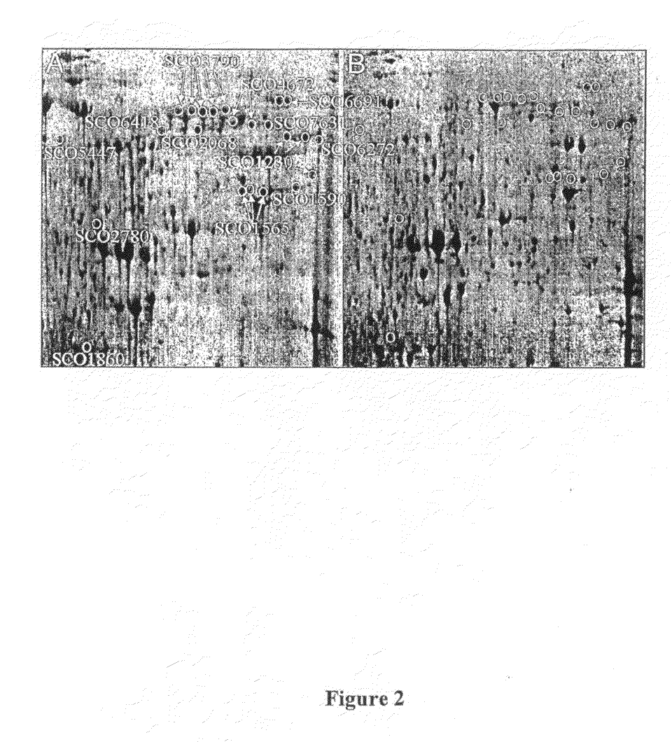

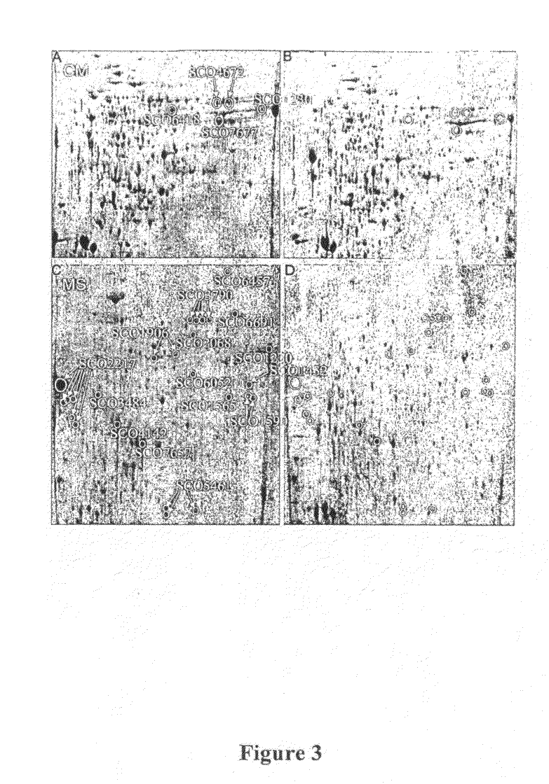

Typical 2D gel electrophoretograms for the cell wall-associated proteome of these strains cultured on R5 medium are shown in FIG. 2 and for MS and CM media in FIG. 3. Significant differences in the staining patterns were observed between the two strains, and proteins that were present in M145 but absent from the tatC strain clearly represented candidates for Tat substrates. Putative Tat-targeted proteins were identified by MALDI-TOF mass spectrometry (after in-gel digestion with trypsin); several of them are marked in FIGS. 2 and 3.

A total of 98 proteins that migrated with unique positions were identified in the M145 washes. In all, 37 of these proteins had identifiable N-terminal signal peptides, and 34 of those signal peptides contained RR dipeptides (Table 3). Cross-referencing with the predicted Tat substrates encoded by the S. coelicolor genome and identified by the programs TatP and TATFIND (Rose et al., (2002) Mol Microbiol 45:943-950; Bendtsen et al., (2005) BMC Bioinformatics 6:167-175), suggested that 21 of this group of 37 had plausible Tat-targeting signal peptides.

In addition to the 37 putative exported proteins, the remaining 61 proteins unique to the tat⁺ strain included ribosomal subunit proteins and proteins of the thioredoxin pathway (Table 5). The identification of such obviously cytoplasmic proteins undoubtedly reflected contamination of the cell wall washes with cytoplasmic proteins from lysed bacteria. Cytoplasmic protein contamination in this type of analysis is almost inevitable given the S. coelicolor life cycle, which involves altruistic lysis of the mycelium to release nutrients for the continued growth of the aerial parts of the colony (Manteca et al., (2006) Res Microbiol 157:143-152). Moreover, such cytoplasmic protein contamination has been reported during analyses of extracellular proteins from B. subtilis (Antelmann et al., (2001) Genome Res 11; 1483-1502) and Mycobacterium tuberculosis (Rosenkrands et al., (2000) Electrophoresis 21:935-948). Indeed, cell lysis, as well as substrate modification (see below) and up-regulation of Sec substrates, also may contribute to the presence of additional extracellular proteins in the ΔtatC strain that are absent in M145 (Table 4). Several of the exported proteins were detected as multiple spots in the M145 sample, possibly as a result of posttranslational modification or proteolysis, and it was not uncommon for one or more of the additional spots for a particular protein to be absent from cell wall fractions of the ΔtatC strain. Therefore, because several putative extracellular proteases are predicted TAT substrates (Dilks et al., (2003) J Bacteriol 185:1478-1483), the lack of a particular protein spot in the 2-DGE analysis of the ΔtatC strain might be indicative of the lack of postexport protein modification rather than a lack of TAT translocation per se.

TABLE 3

Exported proteins observed in the cell wall-associated
fraction of the wild type (M145) strain identified by either
2-DGE and/or MudPIT

| | | | |
|---|---|---|---|
| Sco | Method of | | |
| number | observation | Product | Putative signal peptide |

Twin-arginine signal peptides

| | | | |
|---|---|---|---|
| SCO0432 | 2D | Probable | MRRIRLLAAISAGLALAAGAVAPV |
| TatP | | secreted | PALAS |
| | | peptidase | (SEQ ID NO: 204) |
| SCO0474 | MudPIT | Putative | MEPTVRTHPTRRLPTALVLTAAL |
| | | lipoprotein | LATGCSEQSDDGREPAA |
| | | | (SEQ ID NO: 205) |
| SCO1172 | 2D | Putative | MERERTAPVPTRRRLLKGAALA |
| TatFind | | amidase | TVPYTLLSGTRAAAQ |
| | | (putative | (SEQ ID NO: 206) |
| | | secreted | |
| | | protein) | |
| SCO1196 | 2D | Putative | MALTTRRRALTTLGAALTGAVAL |
| TatP and | | secreted protein | PAGTALAS |
| TatFind | | | (SEQ ID NO: 207) |
| SCO1356 | MudPIT | Putative iron | MTSAPFHPAAGPARRTVVAAAG |
| TatFind | | sulfur protein | AAGLTAVLAACSDSDDGASGD |
| | | (putative | (SEQ ID NO: 208) |
| | | secreted | |
| | | protein) | |
| SCO1432 | 2D and | Putative | MQRRTRPTRTPGGLPLMSRVEQ |
| TatP and | MudPIT | membrane | PSRRALLAAAVAAAAVTGGAIPA |
| Tat Find | | protein. | TARAAS |
| | | | (SEQ ID NO: 209) |
| SCO1565 | 2D and | Putative | MGTQESDERAGGGTGRRALLG |
| TatP and | MudPIT | glycerophosphoryl | AAVLGAGGAVLGLPGTARAA |
| TatFind | | diester | (SEQ ID NO: 210) |
| | | phosphodiesterase | |
| SCO1590 | 2D | Putative | MGGVSRRAFTVAALSAFTLVPE |
| TatP | | secreted protein | ASAA |
| | | (transglycosidase) | (SEQ ID NO: 211) |
| SCO1639 | 2D and | Putative | MRRRSLLIAVPTGLVTLAACGDS |
| TatFind | MudPIT | secreted | DDSGSSSNASESPSPDASATSA |
| | | peptidyl-prolyl | A |
| | | cis-trans | (SEQ ID NO: 212) |
| | | isomeraseprotein | |
| SCO1824 | MudPIT | Secreted | MTAPLSRHRRALAIPAGLAVAAS |
| | | subtilisin- | LAFLPGTPAAATPAAEAA |
| | | like protease | (SEQ ID NO: 213) |
| SCO1906 | 2D | Putative | MSLTRRDFAGRSALTGAGVVLA |
| | | secreted | GSVGALATAPNALAS |
| | | protein (phoX | (SEQ ID NO: 214) |
| | | phosphatase) | |
| SCO1948 | 2D | Putative zinc- | MRRRARSILAVGALLIGGASFAPI |
| | | binding | AQAQ |
| | | carboxypeptidase | (SEQ ID NO: 215) |
| SCO2068 | 2D and | Putative | MTSRHRASENSRTPSRRTVVKA |
| TatP and | MudPIT | secreted | AAAGAVLAAPLAAALPAGAADAA |
| TatFind | | alkaline | P |
| | | phosphatase | (SEQ ID NO: 216) |
| SCO2226 | 2D | Putative bi- | MPATRRTARVRRVAAVTVTALA |
| | | functional | AALLPPLAARAD |
| | | protein | (SEQ ID NO: 217) |
| | | (secreted | |
| | | alpha- | |
| | | amylase/dextrinase) | |
| SCO2286* | 2D | Putative alkaline | MTPANHQAPTSAPSPAPSQSSH |
| | | phosphatase | APELRAAARSLGRRRFLTVTGAA |
| | | | AALAFAVNLPAAGTASAA |
| | | | (SEQ ID NO: 218) |
| SCO2383 | MudPIT | Putative | MRLRRPRRPSDGPPGRSGARR |
| | | secreted | LRVASAPLIPLTVIGAVVAGLIGP |
| | | protein | EPPSDGPLPGTSAVAD |
| | | (PF00652 | (SEQ ID NO: 219) |
| | | Ricin B lectin) | |
| SCO2637 | MudPIT | Putative serine | MTNSPQREPIPGARRAARLALAT |
| | | protease | GLAAALAAVGPVPVALAA |
| | | (pfam00082, | (SEQ ID NO: 220) |
| | | Peptidase_S8, | |
| | | Subtilase | |
| | | family) | |
| SCO2758 | 2D | Beta-N- | MHHSSTAGTGSTAQPSRRSVLT |
| TatP and | | acetylgluco- | ATAVVTAALAAGGGTAYAD |
| TatFind | | saminidase | (SEQ ID NO: 221) |
| | | (putative | |
| | | secreted | |
| | | protein) | |
| SCO2780^{†} | 2D and | Putative | MSHASATHPTRRGILAAGGALGL |
| TatFind | MudPIT | secreted | GAVLAAGGDGDGKSDGA |
| | | protein | (SEQ ID NO: 222) |
| | | (cd01146, | |
| | | FhuD, Fe³⁺- | |
| | | siderophore | |
| | | binding | |
| | | domain FhuD) | |
| SCO2786 | 2D | Beta-N- | MRPHRRHHRTTPRITRLLGSLLL |
| | | acetylhexo- | VAAVGAMTTGAAPVRKAAAE |
| | | saminidase | (SEQ ID NO: 223) |
| | | (Transglycosidase) | |
| SCO2821 | 2D | Putative | MRRPVALRLQAALGTLALAAAA |
| | | secreted | GVVLTMPEAAAAA |
| | | pectate lyase | (SEQ ID NO: 224) |
| SCO3456 | MudPIT | Putative | MRHSGRRVKRATVLAAGAAVAG |
| | | secreted | LLTAACSSPDTTGV |
| | | protein (ABC | (SEQ ID NO: 225) |
| | | transporter | |
| | | substrate | |
| | | binding | |
| | | protein) | |
| SCO3484 | 2D | Putative | MTKPVVPSGVSRRGFLGGSLGV |
| TatP and | | secreted | AGAVLLAACSGGGNSSQGS |
| TatFind | | sugar-binding | (SEQ ID NO: 226) |
| | | protein | |
| SCO3790 | 2D | Conserved | MRKLLPLIGTPSGSHPGGRSAM |
| #TatP and | | hypothetical | TCRFRCGDACFHEVPNTSSNEY |
| TatFind | | protein | VGDVIAGALSRRSMMRAAAVVT |
| | | of PhoX type | VAAAGAGAVGVAGAPSAQAA |
| | | | (SEQ ID NO: 227) |
| SCO4142^{†} | 2D | Phosphate- | MNRRALALGALAVSGALALTAC |
| | | binding | GSDDTGGNSGSDSSSAAAN |
| | | protein | (SEQ ID NO: 228) |
| | | precursor | |
| SCO4152 | 2D and | Putative | MPVTAQPHQPRRRRRTSRLLVV |
| | MudPIT | secreted 5′- | AATVVTAGVLAAAALPASASAG |
| | | nucleotidase | (SEQ ID NO: 229) |
| SCO4672 | 2D and | Putative | MTHTSRRGVLAAFAASAATVPF |
| TatP and | MudPIT | secreted | GGAAAASPARATATAASAVDPA |
| TatFind | | protein | AASAA |
| | | | (SEQ ID NO: 230) |
| SCO4884 | 2D and | Putative | MRRTSRLIRVAVGVASLALAATA |
| | MudPIT | lipoprotein | CGGTSGESGD |
| | | | (SEQ ID NO: 231) |
| SCO4885 | 2D and | Putative | MRRISRITVAGAATASLALALAAC |
| | MudPIT | lipoprotein | GGTSTDSGSE |
| | | | (SEQ ID NO: 232) |
| SCO5461 | 2D | Putative | MITTSLRRRTAAAVLSLSAVLATT |
| | | secreted | AATAPGAAPAPSAAPAKAA |
| | | protein (ADP | (SEQ ID NO: 233) |
| | | ribosylation) | |
| SCO6009 | 2D | Solute-binding | MNTRMRRAAVAVATTAMAVSLA |
| | | protein | ACGSAK |
| | | | (SEQ ID NO: 234) |
| SCO6052 | 2D | Putative | MSTSRNAATRRQVLARTGALGA |
| TatP and | | membrane | GIAFTGALSELFAGTAAAQ |
| TatFind | | protein | (SEQ ID NO: 235) |
| SCO6198^{†} | MudPIT | Putative | MDTTYWSRRRVLTVLGAATAAT |
| TatP and | | secreted | SIPLAAPSRALAA |
| TatFind | | protein | (SEQ ID NO: 236) |
| SCO6272* | 2D | Putative | MTEVSRRKLMKGAAVSGGALAL |
| TatP and | | secreted | PALGAPPATAAPAAGPEDLPGP |
| TatFind | | FAD-binding | AAAAAG |
| | | protein | (SEQ ID NO: 237) |
| SCO6281 | 2D and | Putative FAD- | MSAISRRRFIRHGAVAGGAAVAL |
| TatP and | MudPIT | binding | PALGGWAGEAFAA |
| TatFind | | protein | (SEQ ID NO: 238) |
| SCO6418 | 2D and | Hypothetical | MDRRALMLATGGLLGAAGAAQL |
| TatP and | MudPIT | protein | TAAPSAVAA |
| TatFind | | (pectin lyase | (SEQ ID NO: 239) |
| | | like) | |
| SCO6457 | 2D | Putative beta- | MPHSPVSPAESPAPQPGRPRPV |
| TatP and | | galactosidase | VSRRRLLEGGAAVLGALALSASP |
| TatFind | | (Galactose | LTAQAAVRRAAAD |
| | | mutarotase- | (SEQ ID NO: 240) |
| | | like) | |
| SCO6580 | 2D | Hypothetical | MTPFTDSSRTDAGTDPSADGPG |
| #TatP and | | protein | ESLRRALGVNRRRFLSTCTAVAA |
| TatFind | | (Xylose | GAVAAPVFGASPALAH |
| | | isomerase- | (SEQ ID NO: 241) |
| | | like) | |
| SCO6594 | MudPIT | Putative | MPSSPTPSPTPSGAPEPSGVRR |
| TatP and | | secreted | RSLLAAAAAAPVLASLAGAGTAA |
| TatFind | | protein | AD |
| | | | (SEQ ID NO: 242) |
| SCO6644 | MudPIT | Putative | MRSHRRPRLLAPFLLVPLMAGC |
| | | solute- | FASGGGDDSASGD |
| | | binding | (SEQ ID NO: 243) |
| | | lipoprotein | |
| SCO6691 | 2D | Putative | MAEVNRRRFLQLAGATTAFSAL |
| TatP and | | phospholipase | SASIDRAAAL |
| TatFind | | C | (SEQ ID NO: 244) |
| SCO7399 | 2D | Possible | MRRLLLTAAATTAAALTLAACGT |
| | | binding- | TEPAAD |
| | | protein- | (SEQ ID NO: 245) |
| | | dependent | |
| | | transport | |
| | | lipoprotein | |
| | | (FhuD). | |
| SCO7631 | 2D | Putative | MSTEEQSSGRRHPDRRVLLRAA |
| TatP | | secreted | VAVPAAGAAVAGTAAVPAQAAD |
| | | protein | AA |
| | | | (SEQ ID NO: 246) |

Non twin-arginine signal peptides

| | | | |
|---|---|---|---|
| SCO0930 | MudPIT | Putative | MKTSWRSASLVAGAAALLALTTA |
| | | lipoprotein | CGQDGGGPTGSQNVGATAA |
| | | | (SEQ ID NO: 247) |
| SCO1396 | MudPIT | Putative D- | MTRLSAALRGLATTFAALLAVTA |
| | | alanyl-D- | VPAASAPATAQAR |
| | | alanine | (SEQ ID NO: 248) |
| | | dipeptidase | |
| SCO1968^{†} | 2D and | Putative | MHVRAVAVTTTALLGVALTAPLS |
| | MudPIT | secreted | HARAD |
| | | hydrolase | (SEQ ID NO: 249) |
| SCO3967 | MudPIT | Conserved | MKASRIAAVGAVSGIAVLALSAP |
| | | Hypothetical | AFAHVSVQPEGAAAK |
| | | membrane | (SEQ ID NO: 250) |
| | | protein | |
| SCO4010 | MudPIT | Putative | MFQVTRTPAARLLGAAALSAAAL |
| | | secreted | ASVCAAPSAAVAGAG |
| | | protein | (SEQ ID NO: 251) |
| | | (subtilisin | |
| | | inhibitor) | |
| SCO5074 | 2D and | Putative | MTSSLHHAIRLTTASAIALGGLVT |
| | MudPIT | dehydratase | LGTSAHAA |
| | | | (SEQ ID NO: 252) |
| SCO6738 | 2D | Putative | MNRLRTLTATTAALAASLLVPSLL |
| | | Carboxypeptidase | TAPAHAD |
| | | (putative | (SEQ ID NO: 253) |
| | | secreted | |
| | | protein) | |

Proteins are listed by SCO number and the signal peptide sequences are given in the right column. Twin-arginine dipeptides, where present, are shown in bold. Where multiple arginine dipeptides are present, only the most plausible one is marked. Method of observation indicates which technique was used to identify a particular protein (2D is two-dimensional gel electrophoresis; MudPIT is multidimensional protein identification technology).

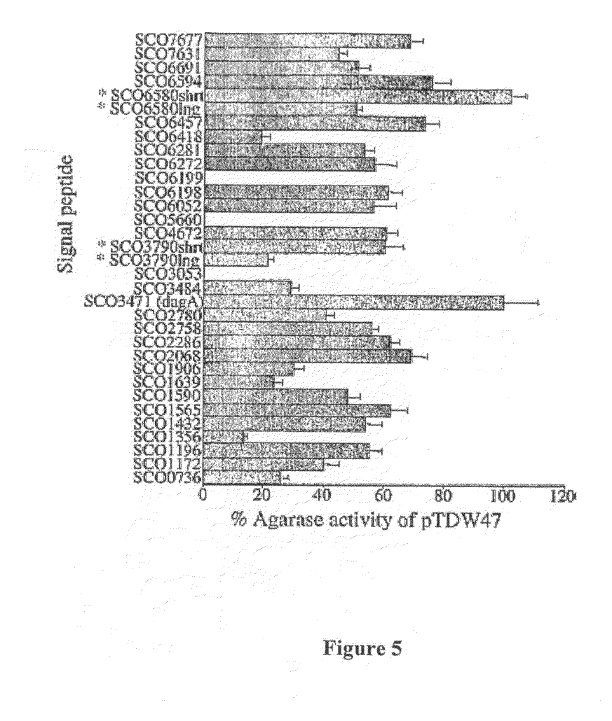

There is some ambiguity about the assigned start codons for SCO3790 and SCO6580. For each of these proteins there is an alternative GTG start codon within the predicted signal peptide coding sequences. These are shown as valine residues that are highlighted in bold underline. Signal peptides initiating at these alternative start codons were tested in the agarase export assay (FIG. 5) and in each case shown to mediate higher levels of agarase export than the full length signal peptides.

* These signal peptides have been shown to mediate Tat-dependent export when fused to the mature sequence of S. lividans xylanase C (Li H, Jacques P E, Ghinet M G, Brzezinski R, Morosoli R (2005) Microbiology 151: 2189-2198).
\ These proteins have also been identified in the extracellular fraction of S. coelicolor grown in liquid medium (Kim D W, Chater K, Lee K J, Hesketh, A (2005) J Bacteriol 187: 2957-2966).
# These proteins are only detected by TatP and TatFind when truncated in silico at the N-terminus to the underlined residue.

The annotation TatFind and/or TatP provided for SCO numbers indicates whether a particular protein has been identified as a putative Tat substrate by either of the two prediction programs TatFind 1.4 or TatP1.0 (information regarding the programs can be found on the world wide web at signalfind.org and cbs.dtu.dk/services/TatP-1.0, respectively).

TABLE 4

Exported proteins observed in the cell wall-associated fraction of the
mutant tatC strain identified by either 2-DGE and/or MudPIT

| | | | |
|---|---|---|---|
| Sco | Method of | | |
| number | observation | Product | Putative signal peptide |

Twin-arginine signal peptides

| | | | |
|---|---|---|---|
| SCO0131 † | 2D | Putative secreted protein | MFRRTLPVLAAAVGLAA |
| | | (DNase I-like) | AAAGPATAD |
| | | | (SEQ ID NO: 254) |
| SCO0462 | MudPIT | Putative oxidoreductase | MRIAVIGAGGVGGYFGA |
| | (mutual) | | RLAAAGNEVTFVARGG |
| | | | HLAAIRRSGLVVHSPLGE |
| | | | LRTSPDSVVAS |
| | | | (SEQ ID NO: 255) |
| SCO0494 | 2D and | Putative iron-siderophore | MLLRTTRTKPWRRLAAA |
| | MudPIT | binding lipoprotein (FhuD | LSAAALGVGLLAGCGSD |
| | (mutual) | type) | SD |
| | | | (SEQ ID NO: 256) |
| SCO0736 | MudPIT | Putative secreted protein | MGDIRRRGAVALGVTAL |
| TatP and | (mutual) | | VAPLTLALTAAPAQAA |
| TatFind | | | (SEQ ID NO: 257) |
| SCO1230 | 2D and | Putative secreted | LRKSSIRRRATAFGTAGA |
| TatP | Mud PIT | tripeptidylaminopeptidase | LVTATLIAGAVSAPAASA |
| | (mutual) | | A |
| | | | (SEQ ID NO: 258) |
| SCO1725 | MudPIT | Putative secreted hydrolase | MRRFRLVGFLSSLVLAA |
| | (mutual) | (similar to scabies esterase) | GAALTGAATAQAA |
| | | | (SEQ ID NO: 259) |
| SCO1860 | 2D and | Putative secreted protein | MSLMRAPHACLTGGPTT |
| TatP | MudPIT | | LNGNTFRMPARRLATVA |
| | (mutual) | | AATALAAGPATLAGAGS |
| | | | AHAT |
| | | | (SEQ ID NO: 260) |
| SCO2116^{†} | 2D and | Putative secreted protein | LSAEHRRNRNKKRLLVY |
| | MudPIT | similar in parts to bacterial | GVASAVVVAATTGTLALA |
| | (~tatC) | AmpD (amidase) | SPGLLGLDADQAAAA |
| | | | (SEQ ID NO: 261) |
| SCO2150 | MudPIT | Cytochrome C heme- | MKKLSARRRHPLAALVVL |
| | (~tatC) | binding subunit | LLALACTGGLYAAFAPAS |
| | | | KAQAD |
| | | | (SEQ ID NO: 262) |
| SCO2217 | 2D and | Putative secreted protein | MGRNTRKRRTPLATKIVA |
| TatFind | MudPIT | | GAAALAIGGGGLVWN |
| | (mutual) | | (SEQ ID NO: 263) |
| SCO2270 | 2D and | Putative membrane protein | MPVRRRRPTALAAAVAT |
| | MudPIT | | AAALGTTALAALGGATAA |
| | (~tatC) | | SAA |
| | | | (SEQ ID NO: 264) |
| SCO2446 | 2D and | Putative secreted peptidase | MSTGPVRRGAALLSAGL |
| | MudPIT | (pfam00082, Peptidase_S8, | VIALLPAGTSAAQ |
| | (mutual) | Subtilase family) | (SEQ ID NO: 265) |
| SCO2505 | MudPIT | Putative ABC-transporter | MNVRRRRISGIAVTAATA |
| | (mutual) | metal-binding lipoprotein | LGLGTLSACSSDSSAA |
| | | (cd01019, ZnuA, Zinc | (SEQ ID NO: 266) |
| | | binding protein) | |
| SCO2591 | 2D* and | Putative secreted protein | LFTSRARLRTRRSRLLTC |
| | MudPIT | | TALAVAAGMLVTTPAVAA |
| | (~tatC) | | (SEQ ID NO: 267) |
| SCO2920 | MudPIT | Putative secreted protease | MTRRSWTFRTAATTVAF |
| | (mutual) | pfam05547, Peptidase_M6 | AAAAATFSAAGVAQAD |
| | | Immune inhibitor A | (SEQ ID NO: 268) |
| | | peptidase M6 | |
| SCO3540 | 2D | Proteinase (putative | MDTRRTHRRTRTGGTRF |
| | | secreted protein) | RATLLTAALLATACSAGG |
| | | | ASTSAGSPAAKAA |
| | | | (SEQ ID NO: 269) |
| SCO3667 | MudPIT | Putative secreted solute- | MSSRARRIAISVAASLMT |
| TatP | (mutual) | binding protein | FSLASCGMLGASESSD |
| | | | (SEQ ID NO: 270) |
| SCO4584 | 2D and | Putative membrane protein | MAPIDSDDNTTAGAGEE |
| | MudPIT | (lolA lipo-protein sorting | LRAGRRKAARYVVPVAV |
| | (mutual) | protein) | MGVAAATIGLVPALA |
| | | | (SEQ ID NO: 271) |
| SCO5022 | MudPIT | Putative lipoprotein | MNRRRPTLLAALTLTAAA |
| TatFind | (~tatC) | | ALTLSACGS |
| | | | (SEQ ID NO: 272) |
| SCO5260 | MudPIT | Secreted protein | MTARSTRRTTAAHSRLA |
| | (~tatC) | (periplasmic binding protein | AVGAIAVAGALLLTGCGD |
| | | AtrA) | QTENGD |
| | | | (SEQ ID NO: 273) |
| SCO5447 | 2D | Putative neutral zinc | MLSSSTPHRRTSHTTSH |
| | | metalloprotease | TSRRTAHRRAAAVALAG |
| | | | VAALIATAVQSGTATAA |
| | | | (SEQ ID NO: 274) |
| SCO5477^{†} | 2D and | Putative oligopeptide- | MTTQRTSGRRKQALAAA |
| | MudPIT | binding lipoprotein | AVVAALLTTAACGGGGD |
| | (mutual) | | DDSGGG |
| | | | (SEQ ID NO: 275) |
| SCO5650 | MudPIT | Putative membrane protein | MAVLSIISRTASRRGVRA |
| | (~tatC) | | LGVVAASAALALSASGNA |
| | | | LAC |
| | | | (SEQ ID NO: 276) |
| SCO6096 | MudPIT | Putative lipoprotein | MPATSALRRTLAVIAALP |
| | (~tatC) | | LLTLAACGYGSQAK |
| | | | (SEQ ID NO: 277) |
| SCO6108 | 2D and | Esterase fusH | MATLIPKKGSTLLNKGIRT |
| | MudPIT | | RRARGALAGGTVLTAAA |
| | (mutual) | | ALLTAVPAAQAI |
| | | | (SEQ ID NO: 278) |
| SCO6197 | 2D and | Putative secreted protein | MSTTSVNGSRSKRRVLL |
| TatP | MudPIT | | RRALPVCVAAGVASIVVF |
| | (mutual) | | GSGSSPDAAAR |
| | | | (SEQ ID NO: 279) |
| SCO6407 | 2D and | Putative gamma- | MRRPVARNLTVLAVSAA |
| | MudPIT | glutamyltranspeptidase | VVVSVGAAAP |
| | (mutual) | (putative secreted protein) | (SEQ ID NO: 280) |
| SCO6572 | 2D | Putative glycosyl hydrolase | MRSVGAGRWRRVASQV |
| | | (putative secreted protein) | GRSVTVWAVSTLTMLVL |
| | | | AGILPDFRLQSpDGDSAT |
| | | | DIAMTAAVGAGA |
| | | | (SEQ ID NO: 281) |
| SCO6590 | 2D and | Putative secreted esterase | MSSIRRSRSAVLGAVLTA |
| | MudPIT | (trypsin like serine | GSLALSTAAAVAVTGGTP |
| | (mutual) | protease) | VADSD |
| | | | (SEQ ID NO: 282) |
| SCO6608 | 2D and | Putative secreted protein | MKLRKRRLCAATGVVLG |
| | MudPIT | (exopolysaccharide | ALTALAALPPAAVAS |
| | (mutual) | biosynthesis) | (SEQ ID NO: 283) |
| SCO6736 | 2D and | Putative metallopeptidase | MPLRLGAFVHRRFIVPGA |
| | MudPIT | | LGAASLMLAIPASAAA |
| | (mutual) | | (SEQ ID NO: 284) |
| SCO7657 | 2D and | Putative secreted protein | MTRTRTTRLRRPLLTGST |
| TatP | MudPIT | | AVAAMAVTAGLVAATAE |
| | (mutual) | | PAKAA |
| | | | (SEQ ID NO: 285) |
| SCO7677^{†} | 2D and | Putative secreted solute- | MSRQIDRRSFLRRGAAG |
| TatP and | MudPIT | binding protein | AAALAVGPGLLAACSTDE |
| TatFind | (mutual) | | PGSA |
| | | | (SEQ ID NO: 286) |

Non twin-arginine signal peptides

| | | | |
|---|---|---|---|
| SCO0297^{†} | 2D and | Putative secreted protein | MRSTGPVSRIGRSLAAVT |
| | MudPIT | | ATAAAGAVAALGLAASPA |
| | (mutual) | | AAA |
| | | | (SEQ ID NO: 287) |
| SCO0324 | 2D | Putative secreted protein | MSPKSKRTRAALAALVV |
| | | (superfamily Quinoprotein | AAGSLVTAGAARAE |
| | | amine dehydrogenase) | (SEQ ID NO: 288) |
| SCO0472 | MudPIT | Putative secreted protein | LNTSIRNRAVTGTALALA |
| | (mutual) | (Quinoprotein amine | VSAVLTACGGDTSSSD |
| | | dehydrogenase) | (SEQ ID NO: 289) |
| SCO0762^{†} | 2D and | Putative secreted protein | MRNTARWAATLGLTATA |
| | MudPIT | | VCGPLAGASLAS |
| | (mutual) | | (SEQ ID NO: 2890) |
| SCO2008 | MudPIT | Putative branched chain | MPGKGLIVRQRSLIAITAA |
| | (mutual) | amino acid binding protein | LAAGALTLTACGS |
| | | | (SEQ ID NO: 291) |
| SCO2096 | MudPIT | Putative membrane protein | MMSGRARLAMCAWAAT |
| | (~tatC) | (Transglutaminase/Protease- | LMAACALLPLVEPATWIL |
| | | like | QAA |
| | | | (SEQ ID NO: 292) |
| SCO2271 | MudPIT | Hypothetical secreted | MPARLRALVTVLCAAVLA |
| | (~tatC) | protein | ALLPAAAAHAE |
| | | | (SEQ ID NO: 293) |
| SCO2483 | 2d | Putative secreted protein | MHSTTSRGAVLATLLALI |
| | | | GVFATYRALPAGAD |
| | | | (SEQ ID NO: 294) |
| SCO2892 | 2D and | Putative secreted protein | MTRGRDGGAGAPPTKH |
| | MudPIT | | RALLAAIVTLIVAISAAIYA |
| | (mutual) | | GASAD |
| | | | (SEQ ID NO: 295) |
| SCO3053 | 2D and | Putative secreted esterase | MSRFRSRAAVTAGLTAIA |
| | MudPIT | (FusH-like) | VAAGCLVVGPVQAA |
| | (mutual) | | (SEQ ID NO: 296) |
| SCO3176 | 2D | Putative membrane protein | MRPSSLRSPWTRAAVAG |
| | | | AAAAGLAAAGLAGVAHA |
| | | | A |
| | | | (SEQ ID NO: 297) |
| SCO3487 | 2D | Putative hydrolase | MTVHKRACTTPPPRASR |
| | | | SFRVRWPVLIAAACAGLV |
| | | | LATTSPPAVAA |
| | | | (SEQ ID NO: 298) |
| SCO3966 | MudPIT | Putative secreted protein | MRKKTFATAALLAAACLT |
| | (~tatC) | (thioredoxin-like) | LSACGSGADSD |
| | | | (SEQ ID NO: 299) |
| SCO4589 | 2D | Putative aminopeptidase | MQLLPSGRALTAGAVAV |
| | | (putative secreted | VTLMAGGSAAGAASAPV |
| | | protein)(Zn dependent) | HPATPTGTAAAAAA |
| | | | (SEQ ID NO: 300) |
| SCO4891 | MudPIT | Putative secreted protein | MRTRTKLTTVLASGTLAA |
| | (~tatC) | | GLLVGGASSASAG |
| | | | (SEQ ID NO: 301) |
| SCO5113^{†} | 2D and | BldKB, putative ABC | MSILRNRTATAAIVAVAA |
| | MudPIT | transport system lipoprotein | GALTLTACGGGDSDSSA |
| | (mutual) | | K |
| | | | (SEQ ID NO: 302) |
| SCO5232 | MudPIT | Putative sugar transporter | MKRKLIAAIGIAGMMVSIA |
| | (~tatC) | sugar binding protein | ACGG |
| | | | (SEQ ID NO: 303) |
| SCO5420 | 2D and | Cholesterol esterase | MESQVRGGTRWKRFAV |
| | MudPIT | | VMVPSVAATAAIGVALAQ |
| | (~tatC) | | GALAA |
| | | | (SEQ ID NO: 304) |
| SCO5660 | MudPIT | Putative secreted peptidase | MRARSHAVLAASVVLAL |
| | (mutual) | | AAGPLAAPAFAA |
| | | | (SEQ ID NO: 305) |
| SCO5776 | MudPIT | Glutamate binding protein | MKLRKVSAASAAVLALAL |
| | (mutual) | | TATACGG |
| | | | (SEQ ID NO: 306) |
| SCO6109 | 2D and | Putative secreted hydrolase | MFAMPRSARLTALSAALL |
| | MudPIT | (fusH-like) | AGALASSSTPALATAGA |
| | (mutual) | | (SEQ ID NO: 307) |
| SCO6199 | 2D and | Secreted esterase | MHRTRPGGHPRSIRFAT |
| | MudPIT | | LAISLTAGSALMTASPAV |
| | (mutual) | | AVTGPAAAD |
| | | | (SEQ ID NO: 308) |
| SCO6591 | 2D and | Putative secreted protein | MKKRSSFVRVLGAAATA |
| | MudPIT | | GALAWAVLAQGNAGAA |
| | (mutual) | | (SEQ ID NO: 309) |
| SCO6710 | 2D | Putative glycosyl hydrolase | MWAALLAGLLMVLGLQA |
| | | | TSASGQPAKAPAAAAA |
| | | | (SEQ ID NO: 310) |
| SCO6723 | 2D and | Putative oxidoreductase | MALAGAVCAAALAALTAV |
| | MudPIT | | PAQAH |
| | (mutual) | | (SEQ ID NO: 311) |
| SCO7250 | 2D and | Conserved hypothetical | MPALATAALLLPLLGAAP |
| | MudPIT | protein (Amidase_2, N- | SAAAPTAAP |
| | (mutual) | acetylmuramoyl-L-alanine | (SEQ ID NO: 312) |
| | | amidase) | |

The table lists proteins by SCO number and the signal peptide sequences are given in the right column. Method of observation indicates which technique was used to identify a particular protein (2D is two-dimensional gel electrophoresis; MudPIT is multidimensional protein identification technology. Bracketed after MudPIT is indicated ‘′tatC’ if the protein was found in the cell-wall fraction of the ′tatC mutant strain only, and ‘mututal’ if it was found in the cell-wall fractions of both the M145 and the ′tatC mutant strains. The annotation TatFind and/or TatP provided for SCO numbers indicates whether a particular protein has been identified as a putative Tat substrate by either of the two prediction programs TatFind or TatP.

* This protein migrated in a unique position on two-dimensional gel analysis of the extracellular fraction from the M145 strain. However it was detected in the tatC mutant strain by MudPIT analysis.
^{\} These proteins have also been identified in the extracellular fraction of S. coelicolor grown in liquid medium (Kim et al., (2005) J Bacteriol 187: 2957-2966).

### Example 5

### Identification of Tat-Dependent Proteins by Multidimensional Protein Identification Technology (MudPIT)

In parallel to the traditional 2-D gel electrophoresis approach, the cell wall proteome of the S. coelicolor strains was analyzed by multidimensional protein identification technology (MudPIT), a sensitive modern technique used to separate and identify even low-abundance proteins from complex mixtures.

Samples of precipitated protein (1 mg) prepared as described above, were dissolved by addition of 100 μl 100 mM Tris-HCl, pH 8.0 200 μl 0.4% RapiGest™ (Waters Ltd, Elstree, herts, UK) in 20 mM Tric-HCl, pH 8.0 10 μl of 40 mM EDTA in water and 40 μl of 45 mMDTT in 100 mM Tris-Hcl pH 8.0. Once the pellets dissolved the samples were heated in a water bath at 90° C. for 30 min then cooled to room temperature. 40 μl of 100 mM iodoacetamide in 100 mM Tris-HCl pH8.0 was added and the sample incubated in the dark for 15 min. 10 μg trypsin (modified sequence grade porcine, Promega, UK) in 10 μl 20, <Tris-HCl pH8.0 was added to each sample which were then incubated at 37° C. After 16 h further 10 μg of trypsin was added and the samples incubated for a further 8 h. The RapiGest was denatured prior to MudPIT mass spectrometry by the addition of 40 μl of 500 mMHCl to give a concentration between 30-50 mM (pH<2) followed by incubation at 37° C. for 45 min. The cloudy samples containing hydrolyzed detergent were centrifuged at 13,000 rpm for 10 min in a BioFuge™ (Heraeus) and the supernatants carefully removed for chromatographic separation.

Samples were loaded onto a biphasic column comprising a strong cation exchange phase (SCX) and a reverse phase. Peptides were eluted stepwise from the SCX phase by using increasing concentrations of salt onto the reverse phase. A reverse-phase gradient then was generated and peptides were eluted into a Q-ToF2 mass spectrometer (Micromass, Manchester, U.K.) The data from each reverse-phase gradient was combined and searched by using MASCOT (Matrix Science, London, U.K. (Perkins et al., Electrophoresis 20:3551-3567 (1999)). The details for the analysis of the samples are as follows.

For chromatographic separation, a 75 μm PicoFrot capillary (NewObjective, Inc.) was packed first with 90 mm of Symmetry C18 300 Å reversephase material (Waters, Ltd, UK) followed by 30 mm of PartiSphere strong cation exchange material (Whatman) using a pressure packing device. The resulting biphasic micro capillary column was equilibrated to 5% acetonitrile/0.1% formic acid. After loading the sample the column was mounted on the Z-spray ion source of a Q-ToF2 mass spectrometer (Micromass, Manchester, UK) and in-line with a capillary HPLC system (CapLC, Water Ltd., UK). A fully automated 9-step chromatography run was carried out, with the mass spectrometer operating in data-dependent modes during each reverse phase elution. The three buffer solutions used for chromatography were 0.1% formic acid (buffer A), 100% acetonitrile/0.1% formic acid (buffer b) and 500 mM ammonium acetate/0.1% formic acid (buffer c). Elution was performed using increasing concentrations of buffer C followed by a reverse-phase gradient. Buffer C concentrations were 0, 10, 25, 50, 75, 100, 200, 300 and 500 mM. The reverse phase gradients consisted of the following profile: t=0 min, 5% B; t=80, 40% B; t=90, 80% B.

The eluted peptides were detected in MS mode in order to select precursor ions for collision-induced dissociation (MS/MS). Every 1.2 s the three most intense signals were subjected to MS/MS analysis. The resulting MS/MS data was deconvoluted using MaxEnt3 (Micromass) and converted into a text file listing the mass, intensity and charge state of the parent ions and mass and intensity of the associated fragment ions using ProteinLynx (Micromass). These data were search using the MASCOT search tool (Matrix Science Ltd, London) against SCODB (the S. coelicolor protein database) using appropriate parameters. The sole fixed modification was carboxyamidomethyl (Cys) and the only variable modification was oxidation (Met). The enzyme was selected as trypsin and the maximum number of missed cleavages was set as 3. Peptide M_{w} tolerance and MS/MS fragment ion tolerance were set as +0.25 Da.

The criteria for protein identification were based on the manufacturer's definitions (Matrix Science, Ltd.) Candidate peptides with probability based Mowse scores exceeding threshold (p<0.05) indicated a significant homology and were referred to as “hits”. Protein scores were derived from peptide ion scores as a non-probabilistic basis for ranking proteins.

MudPIT analysis of the M145 and ΔtatC strains grown on CM medium identified 308 and 279 individual proteins from the cell wall washes, respectively, of which 188 were common to both samples (Tables 3-5). Of the 120 remaining proteins exclusively present in M145 cell wall sample, 20 corresponded to proteins bearing plausible N-terminal signal peptides that contained two consecutive arginines in their sequences (including 11 that had been identified by 2-DGE analysis), strongly suggesting that this group represents the Tat substrates (Table 3).

TABLE 5
Cytoplasmic proteins observed in the extracellular fraction of S. coelicolor
identified by either 2-DGE and/or MudPIT

| Protein | | |
|---|---|---|
| SCO number | Method of observation | Annotation |
| SCO0256 | MudPIT (Mutual) | Putative short chain oxidoreductase |
| SCO0168 | 2D | Possible regulator protein |
| SCO0179 | 2D | Putative zinc-containing |
| | | dehydrogenase |
| SCO0180 | 2D | Conserved hypothetical protein |
| | | SCJ1.29c |
| SCO0199 | 2D | Putative alcohol dehydrogenase |
| SCO0204 | 2D | Putative luxR family two- |
| | | component response regulator |
| SCO0260 | 2D and MudPIT (~tatC) | Conserved hypothetical protein |
| SCO0379^{†} | 2D and MudPIT (Mutual) | Catalase (EC 1.11.1.6) |
| SCO0400 | 2D | Putative epimerase |
| SCO0492 | MudPIT (~tatC) | Putative peptide synthetase |
| SCO0499^{†} | MudPIT (~tatC) | Putative formyltransferase |
| SCO0525 | MudPIT (~tatC) | Conserved hypothetical protein |
| | | SCF11.05 |
| SCO0527 | 2D and MudPIT (mutual) | Cold shock protein |
| SCO0570 | MudPIT (~tatC) | 50S ribosomal protein L33 |
| SCO0592 | 2D | Hypothetical protein SCF55.16c |
| SCO0596 | 2D | Putative DNA-binding protein |
| SCO0597 | 2D | Conserved hypothetical protein |
| | | SCF55.21 c |
| SCO0640 | MudPIT (~tatC) | Conserved hypothetical protein |
| | | SCF56.24c |
| SCO0641 | MUdPIT (mutual) | Tellurium resistance protein |
| SCO0682 | MudPIT (M145) | Hypothetical protein SCF15.03c |
| SCO0714 | 2D | Putative oxidoreductase |
| SCO0740 | 2D | Putative hydrolase |
| SCO0741 | 2D | Putative oxidoreductase |
| SCO0788 | MudPIT (mutual) | Hypothetical protein 3SCF60.20c |
| SCO0797 | 2D | Putative integral membrane protein. |
| SCO0852 | 2D and MudPIT (~tatC) | Putative aldolase |
| SCO0870 | MudPIT (M145) | Putative two-component system |
| | | response regulator |
| SCO0884 | MudPIT (~tatC) | Probable oxidoreductase |
| SCO0885 | MudPIT (~tatC) | Thioredoxin |
| SCO0906 | 2D | Hypothetical protein SCM1.39c |
| SCO0945 | 2D | Putative formamidopyrimidine- |
| | | DNA glycosylase |
| SCO0975 | MudPIT (M145) | 6-phosphogluconate 1- |
| | | dehydrogenase |
| SCO0977 | MudPIT (mutual) | Conserved hypothetical protein |
| SCO0999 | MudPIT (mutual) | Superoxide dismutase |
| SCO1081 | 2D and MudPIT (~tatC) | Putative electron transfer |
| | | flavoprotein, alpha subunit |
| SCO1082 | MudPIT (~tatC) | Putative electron transfer |
| | | flavoprotein, beta subunit |
| SCO1085 | MudPIT (~tatC) | Putative acyltransferase |
| SCO1116 | MudPIT (mutual) | Hypothetical protein |
| SCO1123 | 2D and MudPIT (~tatC) | Hypothetical protein 2SCG38.16 |
| SCO1198 | MudPIT (~tatC) | Putative acyl-CoA dehydrogenase |
| SCO1199 | MudPIT (~tatC) | Putative oxidoreductase |
| SCO1337 | MudPIT (M145) | Putative oxidoreductase |
| SCO1384 | 2D and MudPIT (mutual) | Conserved hypothetical protein |
| SCO1390 | MudPIT (mutual) | Putative PTS system sugar |
| | | phoshotransferase component IIA |
| SCO1391 | MudPIT (M145) | Phosphoenolpyruvate-protein |
| | | phosphotransferase |
| SCO1438 | 2D | ATP phosphoribosyltransferase |
| SCO1453 | MudPIT (~tatC) | Hypothetical protein |
| SCO1462 | MudPIT (M145) | Hypothetical protein SCL6.19c |
| SCO1476 | 2D | S-adenosylmethionine synthetase |
| SCO1480 | MudPIT (mutual) | Conserved hypothetical protein |
| SCO1489 | 2D | Putative DNA-binding protein |
| SCO1496 | 2D | Chorismate synthase |
| SCO1505 | MudPIT (mutual) | 30S ribosomal protein S4 |
| SCO1508 | 2D | Histidyl tRNA synthetase |
| SCO1569 | MudPIT (~tatC) | Putative oxidoreductase |
| SCO1594 | MudPIT (mutual) | Putative phenylalanyl-tRNA |
| | | synthetase beta chain |
| SCO1598 | MudPIT (M145) | 50S ribosomal protein L20 |
| SCO1600 | MudPIT (M145) | Putative translation initiation factor |
| | | IF-3 |
| SCO1620 | MudPIT (~tatC) | Glycine betaine transport system |
| | | permease protein |
| SCO1623 | MudPIT (M145) | Conserved hypothetical protein |
| | | SCI41.06 |
| SCO1626 | MudPIT (mutual) | Putative cytochrome P450 |
| SCO1628 | MudPIT (M145) | Conserved hypothetical protein |
| SCO1643 | MudPIT (mutual) | 20S proteasome alpha-subunit |
| SCO1644 | MudPIT (mutual) | 20S proteasome beta-subunit |
| | | precursor |
| SCO1646 | MudPIT (mutual) | Hypothetical protein |
| SCO1651 | MudPIT (M145) | Conserved hypothetical protein |
| | | SCI41.34c |
| SCO1654 | 2D | Putative two-component response |
| | | regulator |
| SCO1766 | MudPIT (M145) | Putative glycohydrolase |
| SCO1767 | 2D | Putative DNA hydrolase |
| SCO1773 | 2D | Putative L-alanine dehydrogenase |
| SCO1775 | 2D | Conserved hypothetical protein |
| SCO1788 | MudPIT (mutual) | Conserved hypothetical protein |
| SCO1794 | 2D and MudPIT (M145) | Conserved hypothetical protein |
| SCO1809 | MudPIT (mutual) | Conserved hypothetical protein |
| SCO1839 | MudPIT (mutual) | Putative transcriptional regulator |
| SCO1870 | MudPIT (M145) | Hypothetical protein SCI39.17c |
| SCO1921 | MudPIT (mutual) | Putative aminotransferase |
| SCO1922 | MudPIT (mutual) | Putative ABC transporter ATP- |
| | | binding subunit |
| SCO1923 | MudPIT (mutual) | Putative dioxygenase ferredoxin |
| | | subunit |
| SCO1935 | 2D | Transketolase A |
| SCO1936 | MudPIT (mutual) | Putative transaldolase |
| SCO1937 | 2D | Putative glucose-6-phosphate 1- |
| | | dehydrogenase |
| SCO1945^{†} | 2D and MudPIT (mutual) | Triosephosphate isomerase |
| SCO1946 | 2D and MudPIT (mutual) | Phosphoglycerate kinase |
| SCO1947^{†} | 2D and MudPIT (mutual) | Glyceraldehyde-3-phosphate |
| | | dehydrogenase |
| SCO1998 | 2D and MudPIT (mutual) | 30S ribosomal protein S1 |
| SCO2011 | MudPIT (M145) | Putative branched chain amino acid |
| | | transport ATP-binding protein |
| SCO2012 | MudPIT (mutual) | Putative branched chain amino acid |
| | | transport ATP-binding protein |
| SCO2039 | MudPIT (M145) | Indoleglycerol phosphate synthase |
| SCO2045 | 2D | Conserved hypothetical protein |
| SCO2050 | MudPIT (M145) | Phosphoribosylformimino-5- |
| | | aminoimidazole carboxamide |
| | | ribotide isomerase |
| SCO2077 | MudPIT (~tatC) | Hypothetical protein |
| SCO2105 | MudPIT (~tatC) | Putative transcriptional regulatory |
| | | protein |
| SCO2126 | MudPIT (~tatC) | Glucokinase |
| SCO2149 | MudPIT (M145) | Rieske iron-sulfur protein |
| SCO2167 | MudPIT (~tatC) | Hypothetical protein |
| SCO2168 | MudPIT (~tatC) | Hypothetical protein |
| SCO2180^{†} | 2D and MudPIT (mutual) | Putative dihydrolipoamide |
| | | dehydrogenase |
| SCO2181 | 2D and MudPIT (M145) | Putative dihydrolipoamide |
| | | succinyltransferase |
| SCO2183 | MudPIT (~tatC) | Putative pyruvate dehydrogenase E1 |
| | | component |
| SCO2198^{†} | 2D and MudPIT (mutual) | Glutamine synthetase I |
| SCO2206 | MudPIT (M145) | ArsC, arsenate reductase |
| SCO2210 | 2D and MudPIT (M145) | Glutamine synthetase |
| SCO2262 | 2D and MudPIT (M145) | Putative oxidoreductase |
| SCO2265 | MudPIT (~tatC) | Hypothetical protein SCC75A.11c. |
| SCO2339 | MudPIT (mutual) | Hypothetical protein |
| SCO2367 | 2D and MudPIT (mutual) | Conserved hypothetical protein |
| SCO2368 | 2D and MudPIT (mutual) | Conserved hypothetical protein |
| SCO2369 | MudPIT (~tatC) | Putative thiol-specific antioxidant |
| | | protein |
| SCO2370 | MudPIT (mutual) | Conserved hypothetical protein |
| SCO2380 | MudPIT (~tatC) | Conserved hypothetical protein |
| | | SC4A7.08 |
| SCO2382 | MudPIT (~tatC) | Hypothetical protein SC4A7.10 |
| SCO2389 | MudPIT (mutual) | Acyl carrier protein |
| SCO2394 | 2D and MudPIT (M145) | Putative secreted protein |
| SCO2407 | 2D | Putative aldose 1-epimerase |
| SCO2469 | MudPIT (mutual) | Putative reductase |
| SCO2504 | 2D | Glycyl-tRNA synthetase |
| SCO2506 | MudPIT (~tatC) | Putative metal transport ABC |
| | | transporter |
| SCO2529 | 2D | Putative metalloprotease. |
| SCO2548 | MudPIT (mutual) | Putative Hit-family protein. |
| SCO2554 | MudPIT (M145) | DnaJ protein. |
| SCO2576 | MudPIT (~tatC) | Phosphoglycerate mutase |
| SCO2582 | MudPIT (mutual) | Conserved hypothetical protein |
| | | SCC 123.20. |
| SCO2596 | MudPIT (mutual) | 50S ribosomal protein L27 |
| SCO2597 | MudPIT (M145) | Ribosomal protein L21 |
| SCO2612 | 2D and MudPIT (M145) | Nucleoside diphosphate kinase |
| SCO2627 | MudPIT (~tatC) | Putative sugar-phosphate isomerase |
| SCO2633 | 2D and MudPIT (mutual) | Superoxide dismutase [Fe—Zn] (EC |
| | | 1.15.1.1) |
| SCO2634 | MudPIT (mutual) | Conserved hypothetical protein |
| | | SC8E4A.04c |
| SCO2635 | MudPIT (~tatC) | Putative aminopeptidase |
| SCO2643^{†} | MudPIT (mutual) | Aminopeptidase N |
| SCO2696 | MudPIT (M145) | Putative 2-hydroxyacid-family |
| | | dehydrogenase |
| SCO2736^{†} | MudPIT (mutual) | Citrate synthase. |
| SCO2822 | 2D and MudPIT (M145) | Putative decarboxylase |
| SCO2849 | MudPIT (M145) | Conserved hypothetical protein |
| | | SCE20.23. |
| SCO2901 | 2D and MudPIT (mutual) | Hypothetical protein |
| SCO2902 | 2D | Conserved hypothetical protein |
| SCO2904 | 2D and MudPIT (mutual) | Putative ribonuclease PH |
| SCO2905 | MudPIT (mutual) | Hypothetical protein |
| SCO2911 | MudPIT (mutual) | Conserved hypothetical protein |
| SCO2950 | MudPIT (mutual) | DNA-binding protein Hu (hs1) |
| SCO2951 | 2D and MudPIT (mutual) | Putative malate oxidoreductase |
| SCO2999 | MudPIT (~tatC) | Conserved hypothetical protein |
| SCO3023 | MudPIT (M145) | Adenosylhomocysteinase |
| SCO3027 | MudPIT (mutual) | Hypothetical protein SCD84.08c |
| SCO3096^{†} | 2D and MudPIT (mutual) | Enolase |
| SCO3122 | MudPIT (M145) | Putative nucleotidyltransferase |
| SCO3123^{†} | MudPIT (~tatC) | Ribose-phosphate |
| | | pyrophosphokinase |
| SCO3124 | MudPIT (mutual) | Ribosomal L25p family protein |
| SCO3127 | MudPIT (mutual) | Phosphoenolpyruvate carboxylase |
| SCO3182 | MudPIT (M145) | UTP-glucose-1-phosphate |
| | | uridylyltransferase |
| SCO3187 | MudPIT (~tatC) | Conserved hypothetical protein |
| | | SCE22.04 |
| SCO3303 | MudPIT (~tatC) | Putative lysyl-tRNA synthetase |
| SCO3304 | MudPIT (~tatC) | Putative arginyl-tRNA synthetase |
| SCO3328 | MudPIT (M145) | Hypothetical protein |
| SCO3337 | 2D | Pyrroline-5-carboxylate reductase |
| SCO3345 | 2D | Dihydroxy acid dehydratase |
| SCO3373 | MudPIT (M145) | Putative Clp-family ATP-binding |
| | | protease |
| SCO3407 | 2D | Conserved hypothetical protein |
| SCO3409 | MudPIT (mutual) | Putative inorganic pyrophosphatase |
| SCO3425 | MudPIT (M145) | Putative 30S ribosomal protein S18 |
| SCO3427 | MudPIT (M145) | Putative 50S ribosomal protein L31 |
| SCO3429 | MudPIT (mutual) | Putative 50S ribosomal protein L28 |
| SCO3438 | MudPIT (M145) | Hypothetical protein |
| SCO3443 | 2D | Putative pyridine nucleotide- |
| | | disulphide oxidoreductase |
| SCO3463 | MudPIT (M145) | Putative phosphorylase |
| SCO3490 | 2D | Transposase |
| SCO3549 | MudPIT (M145) | Putative anti-sigma factor |
| | | antagonist |
| SCO3549 | 2D and MudPIT (~tatC) | putative anti-sigma factor antagonist |
| SCO3569 | MudPIT (mutual) | Putative endonuclease |
| SCO3571 | 2D | Putative transcriptional regulator |
| SCO3575 | MudPIT (M145) | Conserved hypothetical protein |
| SCO3617 | MudPIT (M145) | Hypothetical protein |
| SCO3622 | 2D | Putative aminotransferase |
| SCO3629 | 2D and MudPIT (M145) | Adenylosuccinate synthetase |
| SCO3647 | 2D and MudPIT (M145) | Conserved hypothetical protein |
| SCO3649^{†} | 2D and MudPIT (mutual) | Putative fructose 1,6-bisphosphate |
| | | aldolase |
| SCO3659 | MudPIT (~tatC) | Hypothetical protein |
| SCO3661 | MudPIT (M145) | ATP-dependent protease ATP-binding subunit |
| SCO3671 | MudPIT (mutual) | Heat shock protein 70 |
| SCO3731 | MudPIT (mutual) | Cold-shock protein |
| SCO3734 | MudPIT (M145) | Hypothetical protein |
| SCO3748 | MudPIT (mutual) | Cold shock protein |
| SCO3767 | 2D and MudPIT (mutual) | Conserved hypothetical protein |
| SCO3790^{†} | 2D | Conserved hypothetical protein |
| SCO3792^{†} | MudPIT (mutual) | Putative methionyl tRNA synthetase |
| SCO3793 | MudPIT (M145) | Conserved hypothetical protein |
| SCO3823 | 2D | Putative quinone oxidoreductase |
| SCO3834 | MudPIT (mutual) | Putative 3-Hydroxyacyl-CoA |
| | | dehydrogenase |
| SCO3877 | MudPIT (~tatC) | Putative 6-phosphogluconate |
| | | dehydrogenase |
| SCO3878 | MudPIT (mutual) | DNA polymerase III, beta chain |
| SCO3889 | MudPIT (mutual) | Thioredoxin |
| SCO3890 | MudPIT (M145) | Thioredoxin reductase (NADPH) |
| SCO3899 | 2D and MudPIT (M145) | Hypothetical protein |
| SCO3906 | MudPIT (mutual) | Putative 30S ribosomal protein S6 |
| SCO3907 | 2D and MudPIT (M145) | Putative single-strand DNA-binding protein |
| SCO3909 | MudPIT (mutual) | 50S ribosomal protein L9 |
| SCO3913 | 2D | Conserved hypothetical protein |
| SCO3917 | MudPIT (M145) | Conserved hypothetical protein |
| SCO3920 | MudPIT (~tatC) | Putative cystathionine/methionine |
| | | gamma-synthase/lyase |
| SCO3956 | MudPIT (~tatC) | Putative ABC transporter ATP- |
| | | binding protein |
| SCO3974 | MudPIT (~tatC) | Hypothetical protein |
| SCO4036 | MudPIT (mutual) | Hypothetical protein |
| SCO4039 | MudPIT (mutual) | Hypothetical protein 2SCD60.05c |
| SCO4041 | MudPIT (M145) | Putative uracil |
| | | phosphoribosyltransferase |
| SCO4043 | MudPIT (~tatC) | Conserved hypothetical protein |
| SCO4048 | MudPIT (M145) | Conserved hypothetical protein |
| SCO4068 | 2D | Phosphoribosylamine-glycine ligase |
| | | (EC 6.3.4.13) |
| SCO4086 | MudPIT (M145) | Amidophosphoribosyltransferase |
| SCO4087 | 2D and MudPIT (mutual) | Phosphoribosylformylglycinamidine |
| | | cyclo-ligase |
| SCO4089^{†} | MudPIT (~tatC) | Valine dehydrogenase (EC 1.4.1.—) |
| SCO4091 | MudPIT (mutual) | Putative DNA-binding protein |
| SCO4109 | 2D and MudPIT (mutual) | Putative oxidoreductase |
| SCO4164 | MudPIT (mutual) | Putative thiosulfate sulfurtransferase |
| SCO4165 | MudPIT (mutual) | Conserved hypothetical protein |
| SCO4173 | MudPIT (~tatC) | Hypothetical protein SCD66.10c |
| SCO4175 | 2D and MudPIT (~tatC) | Hypothetical protein SCD66.12c |
| SCO4199 | MudPIT (M145) | Hypothetical protein |
| SCO4228 | 2D | Putative phosphate transport system |
| | | regulator |
| SCO4240 | 2D | ABC transporter ATP-binding |
| | | protein |
| SCO4246 | MudPIT (M145) | Hypothetical protein SCD8A.19c |
| SCO4277 | 2D and MudPIT (mutual) | Putative tellurium resistance protein |
| SCO4294 | MudPIT (~tatC) | Conserved hypothetical protein |
| SCO4295 | MudPIT (M145) | Cold shock protein |
| SCO4296 | 2D and MudPIT (mutual) | Chaperonin 2 |
| SCO4366^{†} | 2D and MudPIT (M145) | Putative phosphoserine Aminotransferase |
| SCO4411 | MudPIT (mutual) | Putative calcium binding protein |
| SCO4441 | 2D | Putative DNA-binding protein |
| SCO4505 | 2D and MudPIT (mutual) | Cold shock protein |
| SCO4509 | MudPIT (mutual) | Hypothetical protein SCD35.16 |
| SCO4580 | MudPIT (mutual) | Putative fumarylacetoacetase |
| SCO4614 | 2D and MudPIT (mutual) | Conserved hypothetical protein |
| SCO4645 | MudPIT (mutual) | Aspartate aminotransferase |
| SCO4649 | MudPIT (mutual) | 50S ribosomal protein L1 |
| SCO4652 | 2D and MudPIT (mutual) | 50S ribosomal protein L10 |
| SCO4653 | MudPIT (mutual) | 50S ribosomal protein L7/L12 |
| SCO4655 | MudPIT (~tatC) | DNA-directed RNA polymerase |
| | | beta′ chain (fragment) |
| SCO4659 | MudPIT (mutual) | 30S ribosomal protein S12 |
| SCO4660 | MudPIT (mutual) | 30S ribosomal protein S7 |
| SCO4662 | 2D and MudPIT (mutual) | Elongation factor TU-1 |
| SCO4675 | MudPIT (mutual) | Conserved hypothetical protein |
| | | SCD40A.21 c |
| SCO4687 | MudPIT (mutual) | Hypothetical protein SCD31.12c |
| SCO4701 | MudPIT (M145) | 30S ribosomal protein S10 |
| SCO4702 | MudPIT (mutual) | 50S ribosomal protein L3 |
| SCO4703 | 2D and MudPIT (mutual) | 50S ribosomal protein L4 |
| SCO4704 | MudPIT (mutual) | 50S ribosomal protein L23 |
| SCO4705 | MudPIT (mutual) | 50S ribosomal protein L2 |
| SCO4706 | MudPIT (mutual) | 30S ribosomal protein S19 |
| SCO4707 | MudPIT (M145) | 50S ribosomal protein L22 |
| SCO4709 | MudPIT (M145) | 50S ribosomal protein L16 |
| SCO4710 | MudPIT (M145) | 50S ribosomal protein L29 |
| SCO4711 | MudPIT (M145) | 30S ribosomal protein S17 |
| SCO4713 | MudPIT (mutual) | 50S ribosomal protein L24 |
| SCO4714 | MudPIT (M145) | 50S ribosomal protein L5 |
| SCO4716 | MudPIT (mutual) | 30S ribosomal protein S8 |
| SCO4717 | MudPIT (mutual) | 50S ribosomal protein L6 |
| SCO4718 | MudPIT (mutual) | 50S ribosomal protein L18 |
| SCO4719 | MudPIT (mutual) | 30S ribosomal protein S5 |
| SCO4720 | MudPIT (mutual) | 50S ribosomal protein L30 |
| SCO4721 | MudPIT (mutual) | 50S ribosomal protein L15 |
| SCO4723 | MudPIT (M145) | Adenylate kinase |
| SCO4725 | MudPIT (mutual) | Translational initiation factor IF1 |
| SCO4726 | MudPIT (M145) | 50S ribosomal protein L36 |
| SCO4727 | MudPIT (mutual) | 30S ribosomal protein S13 |
| SCO4729 | 2D and MudPIT (mutual) | DNA-directed RNA polymerase |
| | | alpha chain |
| SCO4730 | MudPIT (mutual) | 50S ribosomal protein L17 |
| SCO4734 | MudPIT (mutual) | 50S ribosomal protein L13 |
| SCO4735 | MudPIT (mutual) | 30S ribosomal protein S9 |
| SCO4761 | 2D and MudPIT (mutual) | 10 kD chaperonin cpn10 |
| SCO4762 | MudPIT (mutual) | 60 kD chaperonin cpn60 |
| SCO4770 | 2D and MudPIT (M145) | Inosine 5′ monophosphate |
| | | dehydrogenase |
| SCO4771^{†} | 2D | Putative inosine-5′-monophosphate |
| | | dehydrogenase |
| SCO4800 | MudPIT (~tatC) | Isobutiryl CoA mutase, small |
| | | subunit |
| SCO4808 | MudPIT (mutual) | Succinyl-CoA synthetase beta chain |
| SCO4809^{†} | MudPIT (mutual) | Succinyl CoA synthetase alpha |
| | | chain |
| SCO4814 | MudPIT (M145) | Bifunctional purine biosynthesis protein |
| SCO4824 | 2D and MudPIT (mutual) | Bifunctional protein |
| | | (methylenetetrahydrofolate |
| | | dehydrogenase and |
| | | methenyltetrahydrofolate cyclohydrolase. |
| SCO4827^{†} | MudPIT (mutual) | Malate dehydrogenase |
| SCO4855 | MudPIT (M145) | Putative succinate dehydrogenase |
| | | iron-sulfur subunit |
| SCO4856 | MudPIT (~tatC) | Putative succinate dehydrogenase |
| | | flavoprotein subunit |
| SCO4860 | MudPIT (mutual) | Putative secreted hydrolase |
| SCO4907 | 2D | Transcriptional regulatory protein |
| SCO4921 | MudPIT (M145) | Putative acyl-CoA carboxylase |
| | | complex A subunit |
| SCO4932 | MudPIT (~tatC) | Histidine ammonia-lyase |
| SCO4956 | MudPIT (mutual) | Putative peptide methionine |
| | | sulfoxide reductase |
| SCO4958^{†} | 2D and MudPIT (M145) | Cystathionine gamma-synthase |
| SCO4990 | MudPIT (M145) | Conserved hypothetical protein |
| SCO5031 | MudPIT (mutual) | Alkyl hydroperoxide reductase |
| | | system hypothetical protein |
| SCO5042 | MudPIT (~tatC) | Fumarate hydratase C |
| SCO5059 | 2D | Polyphosphate glucokinase |
| SCO5075 | 2D | Putative oxidoreductase |
| SCO5077 | MudPIT (mutual) | Hypothetical protein |
| SCO5079 | 2D | Conserved hypothetical protein |
| SCO5101 | MudPIT (M145) | Conserved hypothetical protein |
| SCO5102 | MudPIT (mutual) | Putative mutT-like protein |
| SCO5135 | 2D and MudPIT (mutual) | Ferredoxin |
| SCO5145 | MudPIT (mutual) | Conserved hypothetical protein |
| SCO5167 | 2D | Conserved hypothetical protein |
| SCO5169 | MudPIT (mutual) | Putative ATP-binding protein |
| SCO5187 | 2D and MudPIT (mutual) | Putative glutaredoxin-like protein |
| SCO5199 | MudPIT (~tatC) | Conserved hypothetical protein |
| SCO5212^{†} | MudPIT (mutual) | 3-phosphoshikimate 1- |
| | | carboxyvinyltransferase |
| SCO5217 | MudPIT (~tatC) | Anti-sigma factor |
| SCO5221 | 2D | Putative polypeptide deformylase |
| SCO5236 | MudPIT (M145) | Putative glucosamine phosphate |
| | | isomerase |
| SCO5249 | 2D | Putative nucleotide-binding protein |
| SCO5251 | 2D | Acetyltransferase |
| SCO5254 | 2D | Superoxide dismutase |
| SCO5261 | MudPIT (M145) | Putative malate oxidoreductase |
| SCO5262^{†} | MudPIT (~tatC) | Putative dehydrogenase |
| SCO5281 | MudPIT (~tatC) | Putative 2-oxoglutarate |
| | | dehydrogenase |
| SCO5359 | MudPIT (mutual) | 50S ribosomal protein L31 |
| SCO5362 | MudPIT (~tatC) | Conserved hypothetical protein |
| SCO5370 | 2D | ATP synthase delta chain |
| SCO5371 | 2D | ATP synthase alpha chain |
| SCO5373 | MudPIT (~tatC) | ATP synthase beta chain |
| SCO5374 | MudPIT (~tatC) | ATP synthase epsilon chain |
| SCO5396 | 2D | Putative cellulose-binding protein |
| SCO5398 | MudPIT (M145) | Conserved hypothetical protein |
| SCO5405 | 2D | Putative transcriptional regulator |
| SCO5444 | MudPIT (~tatC) | Putative glycogen phosphorylase |
| SCO5464 | MudPIT (mutual) | Possible calcium binding protein |
| SCO5465 | MudPIT (mutual) | Conserved hypothetical protein |
| SCO5470 | MudPIT (mutual) | Serine hydroxymethyltransferase |
| SCO5490 | MudPIT (~tatC) | Conserved hypothetical protein |
| SCO5514 | MudPIT (mutual) | Acetolactate synthase small subunit |
| SCO5515 | MudPIT (mutual) | Probable D-3-phosphoglycerate |
| | | dehydrogenase |
| SCO5522 | 2D and MudPIT (M145) | 3-isopropylmalate dehydrogenase |
| SCO5545 | MudPIT (mutual) | Hypothetical protein SC1C2.26 |
| SCO5547 | 2D | Glutamyl-tRNA synthetase |
| SCO5553 | MudPIT (~tatC) | 3-isopropylmalate dehydratase large |
| | | subunit |
| SCO5561 | MudPIT (mutual) | Hypothetical protein SC7A1.05c |
| SCO5564 | MudPIT (M145) | Putative 50S ribosomal protein L28 |
| SCO5570 | 2D and MudPIT (M145) | Hypothetical protein |
| SCO5571 | MudPIT (~tatC) | 50S ribosomal protein L32 |
| SCO5584 | 2D and MudPIT (M145) | Nitrogen regulatory protein P-II |
| SCO5591 | MudPIT (mutual) | 30S ribosomal protein S16 |
| SCO5607 | MudPIT (~tatC) | Putative transcriptional regulator |
| SCO5624 | MudPIT (M145) | 30S ribosomal protein S2 |
| SCO5651 | MudPIT (M145) | Hypothetical protein SC6A9.16 |
| SCO5655 | 2D | Putative aminotransferase |
| SCO5699 | 2D | Prolyl tRNA synthetase |
| SCO5703 | 2D | Hypothetical protein SC5H4.27 |
| SCO5723 | MudPIT (~tatC) | Putative regulator, BldB |
| SCO5736 | MudPIT (mutual) | 30S ribosomal protein S15 |
| SCO5737^{†} | 2D and MudPIT (mutual) | Guanosine pentaphosphate |
| | | synthetase/polyribonucleotide |
| | | nucleotidyltransferase |
| SCO5744 | 2D | Putative dihydrodipicolinate |
| | | synthase |
| SCO5745 | 2D | Conserved hypothetical protein |
| | | SC9A10.09 |
| SCO5759 | MudPIT (~tatC) | Hypothetical protein SC7C7.14 |
| SCO5777 | MudPIT (M145) | Glutamate uptake system ATP- |
| | | binding protein |
| SCO5806 | MudPIT (~tatC) | Conserved hypothetical protein |
| | | SC4H2.27c |
| SCO5838 | MudPIT (~tatC) | Putative protease |
| SCO5841 | MudPIT (mutual) | Phosphocarrier protein hpr |
| SCO5843 | 2D | Hypothetical protein SC9B10.10 |
| SCO5853 | 2D | Conserved hypothetical protein |
| | | SC9B 10.20c |
| SCO5862 | 2D | Two-component regulator CutR |
| SCO5971 | MudPIT (~tatC) | Conserved hypothetical protein |
| SCO5976 | MudPIT (M145) | Ornithine carbamoyltransferase |
| SCO5984 | 2D | Putative acyl-CoA dehydrogenase |
| SCO5999^{†} | 2D and MudPIT (mutual) | Aconitase |
| SCO6031 | MudPIT (mutual) | Uroporphyrinogen decarboxylase |
| SCO6042 | MudPIT (~tatC) | Conserved hypothetical protein |
| | | SC1B5.02 |
| SCO6046 | MudPIT (M145) | Hypothetical protein |
| SCO6061 | MudPIT (M145) | Putative oxidoreductase |
| SCO6084 | 2D | Putative DNA polymerase |
| SCO6145 | 2D | Hypothetical protein SC1A9.09 |
| SCO6176 | 2D and MudPIT (mutual) | Conserved hypothetical protein |
| SCO6206 | 2D | Putative oxidoreductase |
| SCO6283 | MudPIT (M145) | Conserved hypothetical protein |
| SCO6324 | 2D | Putative hydrolase |
| SCO6409 | MudPIT (mutual) | Methionine aminopeptidase |
| SCO6412 | 2D | Putative aminotransferase |
| SCO6415 | 2D | Putative D-hydantoinase |
| SCO6482 | 2D and MudPIT (mutual) | Conserved hypothetical protein |
| SCO6488 | MudPIT (M145) | Putative acyl-peptide hydrolase |
| SCO6531 | 2D and MudPIT (mutual) | Putative ATP/GTP binding protein |
| SCO6549 | MudPIT (mutual) | Hypothetical protein SC5C7.34 |
| SCO6551 | MudPIT (mutual) | Putative oxidoreductase |
| SCO6606 | MudPIT (M145) | Conserved hypothetical protein |
| | | SC1F2.03c |
| SCO6650 | 2D | Hypothetical protein SC4G2.24 |
| SCO6658 | MudPIT (M145) | 6-phosphogluconate dehydrogenase |
| SCO6663 | MudPIT (M145) | Transketolase B |
| SCO6753 | 2D | Putative nucleotide sugar-1- |
| | | phosphate transferase |
| SCO6776 | 2D | Conserved hypothetical protein |
| SCO6928 | 2D and MudPIT (~tatC) | Putative O-methyltransferase. |
| SCO7000^{†} | MudPIT (mutual) | Isocitrate dehydrogenase |
| SCO7072 | MudPIT (M145) | Conserved hypothetical protein |
| SCO7271 | MudPIT (~tatC) | Putative ion channel subunit |
| SCO7286 | MudPIT (~tatC) | Putative oxidoreducatse |
| SCO7293 | 2D | Conserved hypothetical protein |
| SCO7481 | 2D and MudPIT (mutual) | Conserved hypothetical protein |
| SCO7510^{†} | 2D and MudPIT (mutual) | Peptidyl-prolyl cis-trans isomerase |
| SCO7511 | 2D | Glyceraldehyde 3-phosphate |
| | | dehydrogenase |
| SCO7647 | MudPIT (~tatC) | Putative calcium binding protein |
| SCO7654 | MudPIT (M145) | Putative oxidoreductase |
| SCO7676 | MudPIT (mutual) | Putative ferredoxin |
| SCO7683 | MudPIT (M145) | Putative non-ribosomal peptide |
| | | synthase |
| SCO7699 | 2D | Putative nucleotide-binding protein |
| SCO7700 | 2D | Putative cyclase |
| SCO7728 | 2D and MudPIT (~tatC) | Conserved hypothetical protein |
| SCO7730 | MudPIT (mutual) | Conserved hypothetical protein |

The table lists proteins by SCO number. Method of observation indicates which technique was used to identify a particular protein. 2D is two-dimensional gel electrophoresis; MudPIT is multidimensional protein identification technology. Bracketed after MudPIT is indicated ‘′tatC’ if the protein was found in the cell-wall fraction of the tatC mutant strain only, ‘M145’ if the protein was found in the cell-wall fraction of the M145 strain only and ‘mutual’ if it was found in the cell-wall fractions of both the M145 and the tatC mutant strains.

^{\} These proteins have also been identified in the extracellular fraction of S. coelicolor grown in liquid medium (Kim D W, Chater K, Lee K J, Hesketh, A (2005) J Bacteriol 187: 2957-2966).

### Example 6

### A Tat Transport Assay Based on Agarase

To identify bona-fide Tat-targeting signal peptides associated with the group of 43 proteins identified by the proteomic assays 2-DGE and MudPIT, a reporter-based assay for Tat transport was designed.

Sec-dependent signal peptides are not normally recognized by the Tat machinery, and Tat-targeted proteins usually are folded and, therefore, are not normally compatible for Sec-dependent export. Thus, a reporter-based assay for Tat transport was designed to address directly whether the group of 43 putative Tat substrates were indeed synthesized with bona fide Tat signal peptides.

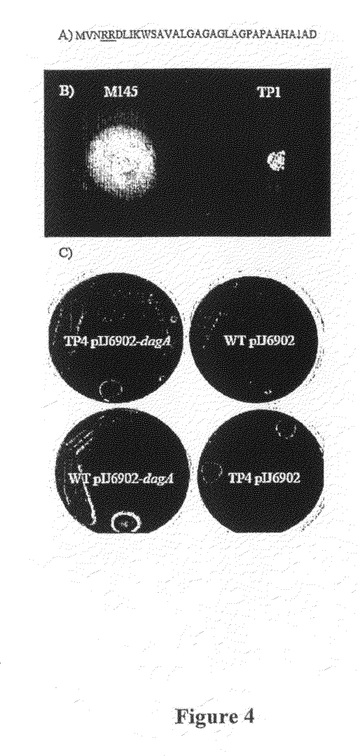

Embedding of S. coelicolor colonies into agar is a phenomenon associated with secretion of the enzyme agarase, which degrades agar to smaller oligosaccharides. As the agar is broken down around the colony, this causes the colony to sink down into the medium. Agarase is encoded by the dagA gene, and the protein product bears an N-terminal signal peptide containing an apparent twin-arginine motif MVNRRDLIKWSAVALGAGAGLAGPAPAAHA↓AD (SEQ ID NO: 313) (FIG. 4A). To determine whether agarase activity could be used in a reporter assay to test the ability of a signal peptide to secrete a protein via the TAT pathway, Applicants tested whether agarase is a TAT-dependent substrate.

a) First, extracellular agarase activity of wild type M145 S. coelicor was compared to that in a tatC mutant (TP1 (ΔtatC::Apra^{R}).

S. coelicor strains M145 and TP1 were grown on MM-C minimal medium for 5 days and were stained with lugol solution.

First, it was seen that colonies of the S. coelicor tat mutants failed to sink into solid media suggesting that DagA is a TAT substrate. Subsequent staining of the agar-containing plates with lugol, an iodine based reagent, showed a clear halo corresponding to the degradation of agar around the wild type strain (M145), while no zone of clearing was observed around colonies of the tat mutant strains (FIG. 4B).

These data provide strong and visual evidence that that the tat mutants have no significant extracellular agarase activity.

b) Second, to ensure that agarase activity was expressed in the tat mutants, the dagA gene was placed under control of the tipA thistrepton-inducible promoter and incorporated onto the chromosome in a single copy at the φC31 attachment site. After integration of the construct into S. coelicolor strain M145 (wild type) and TP4 (ΔtatC), harboring pIJ6902-dagA in a single copy, thiostrepton was added to induce expression of agarase. Transcription of dagA in S. coelicolor is highly regulated and reported to be repressed by glucose in the growth medium (serving-Gonzalez et al., (199) Microbiology 140:2555-2565). The bacteria were grown on minimal medium containing 0.5% glucose, apramycin and thiostrepton for 3 days before being stained with lugol solution.

As shown in FIG. 4C, agarase activity was produced in the wild type strain M145, while no active agarase was secreted in the TP4 (ΔtatC) background.

These data demonstrate that S. coelicolor DagA is a TAT-dependent extracellular protein.

c) Third, to exploit agarase as a screen for Tat-dependent protein export the ability of the DagA mature protein to be targeted via alternative export routes was tested.

The TAT targeting signal peptide of DagA was swapped for the Sec-targeting signal peptides of three S. coelicolor proteins: SCO3053, SCO5660, and SCO6199, and the agarase activity determined as described above.

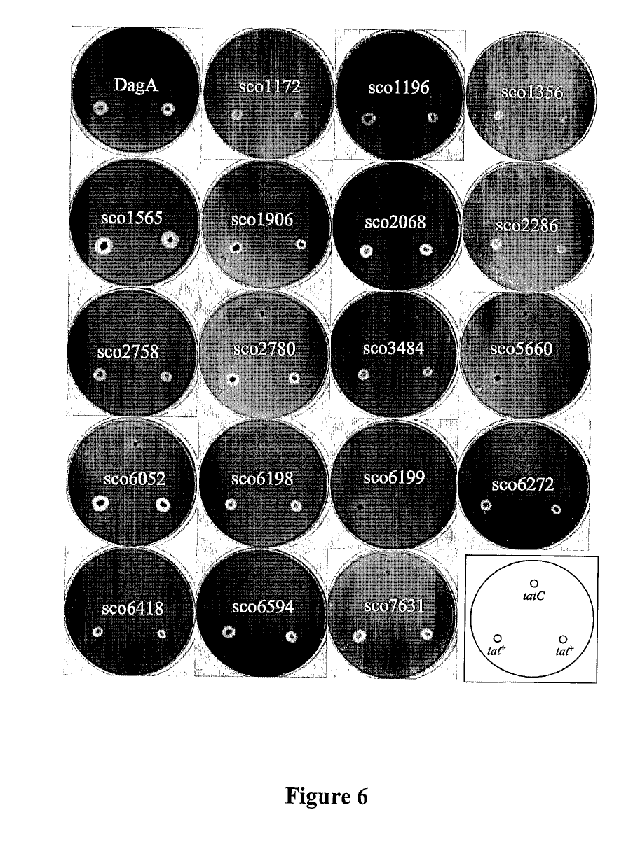

No extracellular agarase activity could be detected when the mature portion of the enzyme was targeted to the Sec machinery (FIG. 5; see FIG. 6), most likely as a result of folding of agarase before export.

Taken together, these data show that DagA can be used as a reporter exclusively for TAT-mediated protein secretion.

### Example 7

### Identification of Bona Fide TAT-Exported Proteins and Signal Peptides Using the TAT Transport Assay Based on Agarase

The two proteomic techniques above identified putative (a total of 43) proteins that could have been transported by the Tat pathway (Table 3). To identify whether these proteins contained bona fide Tat-targeting signal peptides, the reporter-transport assay based on agarase activity was used. Fusions of the signal peptides of each of the putative TAT-targeted proteins to mature agarase were constructed and expressed in a nonagarase-producing host strain (Streptomyces lividans 1326) and assessed for the production of agarase activity using the lugol-based plate test.

Tat dependency of Streptomyces coelicolor agarase was demonstrated by growing strain M145 and TP4 harboring either pIL6902 or pIJ6902dagA on minimal medium containing glucose, which strongly represses expression of the native agarase. Approximately 10⁴ spores of each strain were streaked or spotted onto minimal medium containing glucose, additionally supplemented with apramycin and thiostrepton (to induce expression of the myc-tagged agarase) and grown at 37° C. for 72 hours. And then stained with Lugol solution (Sigma) for 45 min. Plasmids encoding signal peptide-agarase fusion proteins (listed in Table 2) were mated into S. lividans tat⁺ 1326 and 10-164 (as 1326, msiK⁻), and the 10-164 isogenic tatC mutant (2Faury et al., (2004) Biochem Biophys Acta 1699:155-162), all of which do not possess native agarase. Plates containing mature spores of the resultant strains, along with S. coelicolor M145 and TP4 were used to inoculate, with a dissecting needle, MM-C minimal Medium (MM-C) (10 g Agar, 1 g (NH₄)₂SO₄, 0.5 g K₂HPO, 0.2 g FeSO₄.7H₂O in 1 L) lacking a carbon source other than agar. The inoculated plates were grown for 5 days at 30 C and then stained with Lugol solution (Sigma) for 45 min before photography on a light box. Relative activities were estimated by determining the diameter of the zones of clearing using the measure tool of the image manipulation program GIMP (open source software distributed under a GNU general public license at <www.gimp.org>). One sample was included in every batch of results as a standard to which the diameters of the zones of clearing were adjusted. Each zone of clearing was measured twice with each measurement being at right angles to the other. The mean of the two measurements was taken and used to calculate R² (R=radius) as an estimate of the concentration of agarase. All measurements were then expressed as a percentage of the activity of pTDW47, which is the reporter pTDW46 with the native agarase signal peptide reintroduced. Nine measurements were made for each data point and the standard error of the mean was calculated for each point.

(a) Bona Fide Signal Peptides:

In all, 25 of the 43 signal peptides clearly directed Tat-dependent secretion of DagA, consistent with their being bona fide Tat signal peptides (FIG. 5 and Table 6; a representative sample of these also was tested in a S. lividans tatC strain (Faury et al., supra), further demonstrating their Tat dependence; FIG. 6). Of these 25, TATFIND predicted 20 and TatP predicted 18 to be Tat signal peptides. Both prediction programs failed to identify two of the Tat-active signal peptides (SCO2286 and SCO3790) because of their unusual length; however, in silico N-terminal truncation of these signal peptides restored the ability of both programs to recognize them as Tat-targeting signal peptides. Moreover, it is clear when comparing the results from the agarase assays (FIGS. 5 and 6) that secretion efficiency varies significantly with signal peptide primary structure. Thus, comparisons of heterologous protein translocation by using only a single Tat or Sec signal peptide, as recently performed for hTNFalpha and hIL10 in S. lividans (Schaerlaekens et al., (2004) J Biotechnol 112:279-288), may not reflect the true potential of each pathway for protein transport. The remaining 18 signal peptides of the 43 that contained consecutive arginine residues could not mediate secretion of active agarase, and of these, TATFIND predicted 0 and TatP predicted only 1 to be a genuine Tat-targeting signal peptide (Table 3). These 18 signal peptides therefore were considered not to be Tat-targeting signals and the corresponding passenger proteins are not detected in the cell wall fraction of the ΔtatC strain presumably because of pleiotropic effects.

Of the exported proteins identified in cell wall analyses of the ΔtatC strain (Table 4), two (SCO0736 and SCO7677) were predicted by both TATFIND and TatP programs to contain Tat-targeting signal peptides. These signal peptides were subjected to the agarase test, and both conferred extracellular agarase activity to the S. lividans host strain, bringing the total of verified Tat-targeting signal peptides to 27 (FIG. 5). The apparent presence of these final two proteins in the Δ tatC strain cell wall might have been due to minor contamination of washes because of cell lysis. Finally, rare examples of Tat-dependent signal peptides lacking consecutive arginine residues have been reported (e.g., Ignatova et al., (2002) 291:146-149). A total of seven exported proteins were identified in the cell wall fraction of M145 that did not contain obvious twin-arginine motifs in their signal peptides (Table 3). The agarase test was applied to six of these seven signal peptides, but none were found to direct export of active agarase.

(b) TAT-Exported Proteins:

Taken altogether, Applicants have unambiguously identified 27 Tat-dependent proteins in S. coelicolor. This number represents 30% of all of the exported proteins that we detected in the cell wall fraction of the tat⁺ strain and clearly demonstrates that the Tat pathway is a major protein translocation route. The agarase reporter assay developed here and used to validate possible Tat-targeting signal peptides is particularly powerful because it is not only facile and rapid but also semiquantitative and, therefore, provides a ready measure of transport efficiency for a particular Tat-targeting signal peptide. It is also anticipated that the agarase reporter system will facilitate the exploitation of the Tat pathway for heterologous protein production. The identified Tat-exported proteins listed in Table 6 represent a broad spectrum of functional classes: several are predicted to be involved in phosphate and carbohydrate metabolism, nutrient transport, and lipid metabolism. However, unlike the well characterized E. coli system, only 3 of the 27 Tat substrates detected here are likely to be cofactor-containing and, remarkably, 2 of these (SCO6272 and SCO6281) are associated with a type I modular polyketide synthase gene luster, indicating a role in secondary metabolism and, therefore, are not expected a priori to be exported. Substrates of the twin-arginine translocation pathway have hitherto not been found associated with a secondary metabolite gene cluster. Other notable Tat substrates identified here include two proteins involved in peptidoglycan metabolism, SCO0736 and SCO1172, the latter being a probable cell wall amidase. Considering the connectivity between nonexport of amidases and the integrity of the cell envelope in E. coli tat mutants (Ize et al., (2003) Mol Microbiol 48:1183-1193), the identification of SCO1172 as a Tat substrate may well account for the fragility of the Streptomyces tat mutants. Moreover, as with the E. coli model, it may perhaps be possible to “rescue” the fragile phenotype of the Streptomyces tat mutants by increased expression of a Sec-dependent amidase. This likely would enable a further proteomic analysis of the “Tat secretome” in this organism and the assignment of Tat-targeted proteins not associated with the cell wall. A major surprise was that 4 of the 27 Tat substrates are annotated as lipoproteins (Bendtsen et al., (2005) BMC bioinformatics 6:167-175) (Table 6). Again, this in vivo data demonstrates the Tat dependence of putative lipoproteins and implies that class I and class II cellular signal peptidases can recognize and cleave Tat-targeting signal peptides. Interestingly, an outer membrane-localized dimethyl sulfoxide reductase, a Tat substrate that is also strongly predicted to be a lipoprotein, has been described in Shewanella oneidensis (Gralnick et al., (2006) Proc Natl Acad Sci USA 103:4669-4674).

Overall, these data demonstrate that the Tat pathway can export a diverse range of proteins and further reinforces the contention that the Tat pathway is a truly general protein export system in this organism. The ability of the Streptomyces Tat pathway to export proteins requiring various anti- and possibly posttranslocation modifications, as well as two of the largest single Tat substrates ever reported (SCO6198 and SCO6457, at 116 and 146 kDa, respectively), underscores the potential of the Tat system in Streptomycetes for heterologous protein production.

TABLE 6
Tat-dependent proteins identified in S. coelicolor

| Protein | Description |
|---|---|
| SCO0736 | Putative secreted protein |
| SCO1172 | Putative amidase (putative secreted protein) |
| SCO1196 | Putative secreted protein |
| SCO1356*† | Putative iron sulfur protein (putative secreted |
| | protein) |
| SCO1432 | Putative membrane protein |
| SCO1565 | Putative glycerophosphoryl diester |
| | phosphodiesterase |
| SCO1590 | Putative secreted protein (transglycosidase) |
| SCO1639† | Putative secreted peptidyl-prolyl cis-trans |
| | isomerase protein |
| SCO1906 | Putative secreted protein (PhoX phosphatase) |
| SCO2068 | Putative secreted alkaline phosphatase |
| SCO2286 | Putative alkaline phosphatase |
| SCO2758 | β-N-acetylglucosaminidase (putative secreted |
| | protein) |
| SCO2780† | Putative secreted protein (cd01146, FhuD, |
| | Fe3_-siderophore binding domain FhuD) |
| SCO2786 | β-N-acetylhexosaminidase (Transglycosidase) |
| SCO3484† | Putative secreted sugar-binding protein |
| SCO3790 | Conserved hypothetical protein of PhoX type |
| SCO4672 | Putative secreted protein |
| SCO6052 | Putative membrane protein |
| SCO6198 | Putative secreted protein |
| SCO6272* | Putative secreted FAD-binding protein |
| SCO6281* | Putative FAD-binding protein |
| SCO6457 | Putative_-galactosidase (Galactose |
| | mutarotase-like) |
| SCO6580 | Hypothetical protein (Xylose isomerase-like) |
| SCO6594 | Putative secreted protein |
| SCO6691 | Putative phospholipase C |
| SCO7631 | Putative secreted protein |
| SCO7677 | Putative secreted solute-binding protein |

*Cofactor containing.
†Putative lipoproteins.

What is claimed is:

1. An isolated TAT signal peptide comprising the sequence motif (X⁻¹)RR(X⁺²)(X⁺³)(X⁺⁴), wherein R is arginine, X⁻¹ is amino acid M, H, A, P, K, R, N, T, G, S, D, Q or E; X⁺² is amino acid A, P, K, R, N, T, G, S, D, Q or E; X⁺³ is I, W, F, L, V, Y, M, C, H, A, P, N or T; and X⁺⁴ is Q, I, L, V, M or F, and wherein said motif is not within the first 35N′ terminal residues of the amino acid sequence of the polypeptide.

2. An isolated TAT signal peptide comprising the sequence motif (X⁻¹)RR(X⁺²)(X⁺³)(X⁺⁴), wherein R is arginine, X⁻¹ is amino acid H, A, P, K, R, N, T, G, S, D, Q E or L; X⁺² is A, P, K, R, N, T, G, S, D, Q or E; X⁺³ is I, W, F, L, V, Y, M, C, H, A, P, N or T; and X⁺⁴ is T, G or A, and wherein the motif is within the first 35N′ terminal residues of the amino acid sequence of the polypeptide.

3. The TAT signal peptide of claim 2, wherein when X⁻¹ is H then X⁺⁴ is A.

4. The TAT signal peptide of claim 2, wherein when X⁻¹ is L then X⁺⁴ is G.

5. An isolated TAT signal peptide comprising the sequence motif (X⁻¹)RR(X⁺²)(X⁺³) (X⁺⁴), wherein RR represents two adjacent arginine residue and X designates positions restrict to other selected amino acids: X⁻¹ is M, H, A, P, K, R, N, T, G, S, D, Q, or E; X⁺² is a polar amino acid residue; and X⁺³ and X⁺⁴ are non-polar amino acid residues, and wherein the motif is not within the first 35N′ terminal residues of the amino acid sequence of the polypeptide.

6. An isolated variant of the TAT signal peptide of claim 5.

7. An isolated TAT signal peptide comprising the amino acid sequence of the signal peptide of proteins SCO2286 (SEQ ID NO: 218), SCO3790long (SEQ ID NO: 227), SCO6580long (SEQ ID NO:241), SCO1590 (SEQ ID NO: 211), SCO1824 (SEQ ID NO: 213), SCO6580short (SEQ ID NO: 182), or SCO3790short (SEQ ID NO: 122).

8. An isolated polynucleotide comprising a polynucleotide sequence encoding a signal peptide of claims 1, 2, 5, or 6.

9. The isolated polynucleotide of claim 8, wherein said nucleotide sequence encoding a TAT signal polypeptide is operably linked to a second nucleotide sequence encoding a heterologous polypeptide.

10. An expression vector comprising a first nucleotide sequence encoding a TAT signal polypeptide of claims 1, 2, or 5 operably linked to a second nucleotide sequence encoding a heterologous polypeptide.

11. A fusion polypeptide comprising a TAT signal peptide of claim 1, 2, or 5 and a heterologous polypeptide.

12. The fusion polypeptide of claim 11, wherein said TAT signal peptide is the secretory leader sequence of polypeptides that are naturally expressed by Streptomyces.

13. The fusion polypeptide of claim 11, wherein said heterologous polypeptide is an enzyme, a growth factor or a hormone.

14. The fusion polypeptide of claim 13, wherein said enzyme is a protease, a carbohydrase, an isomerase, a glucoamylase, a kinase, an amidase, an esterase, or an oxidase.

15. The fusion polypeptide of claim 11, wherein said heterologous polypeptide is not naturally associated with a secretion signal peptide.

16. A bacterial host cell genetically transformed with an expression vector of claim 10.

17. A method for producing a heterologous polypeptide comprising:
(a) culturing host cells comprising an expression vector comprising a first nucleotide sequence encoding a TAT signal peptide operatively linked to a second nucleotide sequence encoding a heterologous polypeptide,
wherein said TAT signal peptide comprises the sequence motif (X⁻¹)RR(X⁺²)(X⁺³)(X⁺⁴), wherein R is arginine, X⁻¹ is amino acid M, H, A, P, K, R, N, T, G, S, D, Q or E; X⁺² is amino acid A, P, K, R, N, T, G, S, D, Q or E; X⁺³ is I, W, F, L, V, Y, M, C, H, A, P, N or T; and X⁺⁴ is Q, I, L, V, M or F, and wherein said motif is not within the first 35N′ terminal residues of the amino acid sequence of the polypeptide; and
(b) producing said heterologous polypeptide.

18. A method for producing a heterologous polypeptide comprising:
(a) culturing host cells comprising an expression vector comprising a first nucleotide sequence encoding a TAT signal peptide operatively linked to a second nucleotide sequence encoding a heterologous polypeptide,
wherein said TAT signal peptide comprises the sequence motif (X⁻¹)RR(X⁺²)(X⁺³)(X⁺⁴), wherein R is arginine, X⁻¹ is amino acid H, A, P, K, R, N, T, G, S, b, Q E or L; X⁺² is A, P, K, R, N, T, G, S, D, Q or E; X⁺³ is I, W, F, L, V, Y, M, C, H, A, P, N or T; and X⁺⁴ is T, G or A, and wherein the motif is within the first 35N′ terminal residues of the amino acid sequence of the polypeptide; and
(b) producing said heterologous polypeptide.

19. The method of claims 17 or 18, wherein said step of producing comprises recovering said heterologous polypeptide from the culture medium.

20. The method of claim 17 or 18, wherein said host cells are prokaryotic cells.

21. The method of claim 17 or 18, wherein said host cells are Streptomyces bacterial cells.

22. The method of claim 17 or 18, wherein said host cells are S. coelicolor or S. lividans bacterial cells.

23. The method of claim 17 or 18, wherein said heterologous polypeptide is an enzyme, a growth factor or a hormone.


SEQUENCE LISTING (312 sequences)

SEQ ID NO: 1 - DNA, 59 bp, Artificial Sequence
  synthetic primer
acccacccagccgcctcggtgagaaggtaaagacttatgattccggggatccgtcgacc

SEQ ID NO: 2 - DNA, 58 bp, Artificial Sequence
  synthetic primer
atctcgtgcggcgggccggagatcaccggccctgcgtcatgtaggctggagctgcttc

SEQ ID NO: 3 - DNA, 59 bp, Artificial Sequence
  synthetic primer
ccgtggtggcggcccggacatcccaaggagcttcaggtgattccggggatccgtcgacc

SEQ ID NO: 4 - DNA, 58 bp, Artificial Sequence
  synthetic primer
ttcccggcccgcgggccgggcgtgggaggagcggggtcatgtaggctggagctgcttc

SEQ ID NO: 5 - DNA, 59 bp, Artificial Sequence
  synthetic primer
cctgcccgcaacaaggagaaggaccccgaggggcggatgattccggggatccgtcgacc

SEQ ID NO: 6 - DNA, 58 bp, Artificial Sequence
  synthetic primer
gggcggcgacgcgtgccgtcaccgccccggcggtcctcatgtaggctggagctgcttc

SEQ ID NO: 7 - DNA, 36 bp, Artificial Sequence
  synthetic primer
ggcggcaagcttagatctaccgattgtcaccctgcg

SEQ ID NO: 8 - DNA, 38 bp, Artificial Sequence
  synthetic primer
ggccgggaattccatatgcgttctccttcttcgattcc

SEQ ID NO: 9 - DNA, 31 bp, Artificial Sequence
  synthetic primer
ggcggcggatccgcagacctcgaatgggaac

SEQ ID NO: 10 - DNA, 29 bp, Artificial Sequence
  synthetic primer
ggcggctctagatctgttccgtgaggtgc

SEQ ID NO: 11 - DNA, 32 bp, Artificial Sequence
  synthetic primer
ggcggccatatgctatttgccgactaccttgg

SEQ ID NO: 12 - DNA, 32 bp, Artificial Sequence
  synthetic primer
ggcggcggatccctgacgccgttggatacacc

SEQ ID NO: 13 - DNA, 30 bp, Artificial Sequence
  synthetic primer
ggaattccatatggtcaaccgacgtgatct

SEQ ID NO: 14 - DNA, 61 bp, Artificial Sequence
  synthetic primer
gaggatccctacagatcctcctccgagatgagcttctgctccacggcctgatacgtcctga

SEQ ID NO: 15 - DNA, 30 bp, Artificial Sequence
  synthetic oligonucleotide
ggcgcgcatatgagacggatccgcctgttg

SEQ ID NO: 16 - DNA, 33 bp, Artificial Sequence
  synthetic oligonucleotide
ggcgcgggatccggtgctcgcgagtgcgggtac

SEQ ID NO: 17 - protein, 30 aa, Streptomyces coelicolor
MRRIRLLAAISAGLALAAGAVAPVPALAST

SEQ ID NO: 18 - DNA, 29 bp, Artificial Sequence
  synthetic oligonucleotide
ggcgcgcatatggaaccgaccgtccgcac

SEQ ID NO: 19 - DNA, 30 bp, Artificial Sequence
  synthetic oligonucleotide
ggcgcgggatccctcggtcgccgcgggttc

SEQ ID NO: 20 - protein, 42 aa, Streptomyces coelicolor
MEPTVRTHPTRRLPTALVLTAALLATGCSEQSDDGREPAATE

SEQ ID NO: 21 - DNA, 27 bp, Artificial Sequence
  synthetic oligonucleotide
ggccgccatatgctcctcagaacaacg

SEQ ID NO: 22 - DNA, 30 bp, Artificial Sequence
  synthetic oligonucleotide
ggcgcgggatcccacggtgaccgggaaggc

SEQ ID NO: 23 - protein, 57 aa, Streptomyces coelicolor
MLLRTTRTKPWRRLAAALSAAALGVGLLAGCGSDSDDPADEAGGGTPAAAGAFPVTV

SEQ ID NO: 24 - DNA, 28 bp, Artificial Sequence
  synthetic oligonucleotide
ggccgccatatgggggacatacgcagac

SEQ ID NO: 25 - DNA, 30 bp, Artificial Sequence
  synthetic oligonucleotide
ggccgcggatccgcagctcgcggcctgggc

SEQ ID NO: 26 - protein, 35 aa, Streptomyces coelicolor
MGDIRRRGAVALGVTALVAPLTLALTAAPAQAASC

SEQ ID NO: 27 - DNA, 30 bp, Artificial Sequence
  synthetic oligonucleotide
ggcgcgcatatgaagacctcctggcggagc

SEQ ID NO: 28 - DNA, 30 bp, Artificial Sequence
  synthetic oligonucleotide
ggcgcgggatccgccgggggcggccgtggc

SEQ ID NO: 29 - protein, 44 aa, Streptomyces coelicolor
MKTSWRSASLVAGAAALLALTTACGQDGGGPTGSQNVGATAAPG

SEQ ID NO: 30 - DNA, 29 bp, Artificial Sequence
  synthetic oligonucleotide
ggcgcgcatatggaaagggagaggacagc

SEQ ID NO: 31 - DNA, 30 bp, Artificial Sequence
  synthetic oligonucleotide
ggcgcgggatccgtagtcgacggcacggac

SEQ ID NO: 32 - protein, 43 aa, Streptomyces coelicolor
MERERTAPVPTRRRLLKGAALATVPYTLLSGTRAAAQVRAVDY

SEQ ID NO: 33 - DNA, 29 bp, Artificial Sequence
  synthetic oligonucleotide
ggcgcgcatatggccctcaccacccgacg

SEQ ID NO: 34 - DNA, 33 bp, Artificial Sequence
  synthetic oligonucleotide
ggcgcgggatccctgggacgccagcgcggtacc

SEQ ID NO: 35 - protein, 32 aa, Streptomyces coelicolor
MALTTRRRALTTLGAALTGAVALPAGTALASQ

SEQ ID NO: 36 - DNA, 73 bp, Artificial Sequence
  synthetic oligonucleotide
ggccggcatatgaggaagagcagcatacggcggagggcgaccgccttcggcacggccggagcactggtcaccg

SEQ ID NO: 37 - DNA, 76 bp, Artificial Sequence
  synthetic oligonucleotide
ggccggggatccggcgctcgcggcgggtgccgagacggcgccggcgatcagcgtggcggtgaccagtgctccggcc

SEQ ID NO: 38 - protein, 36 aa, Streptomyces coelicolor
MRKSSIRRRATAFGTAGALVTATLIAGAVSAPAASA

SEQ ID NO: 39 - DNA, 28 bp, Artificial Sequence
  synthetic oligonucleotide
ggcgcgcatatgaccagcgcaccgtttc

SEQ ID NO: 40 - DNA, 30 bp, Artificial Sequence
  synthetic oligonucleotide
ggcgcgggatccgccgccgtcgccggacgc

SEQ ID NO: 41 - protein, 45 aa, Streptomyces coelicolor
MTSAPFHPAAGPARRTVVAAAGAAGLTAVLAACSDSDDGASGDGG

SEQ ID NO: 42 - DNA, 27 bp, Artificial Sequence
  synthetic oligonucleotide
ggcgcgcatatgacacgactctccgcc

SEQ ID NO: 43 - DNA, 30 bp, Artificial Sequence
  synthetic oligonucleotide
ggcgcgggatccctcgtgccgtgcctgcgc

SEQ ID NO: 44 - protein, 38 aa, Streptomyces coelicolor
MTRLSAALRGLATTFAALLAVTAVPAASAPATAQARHE

SEQ ID NO: 45 - DNA, 39 bp, Artificial Sequence
  synthetic oligonucleotide
ggcgcgcatatgcaacgacggacccgcccgacccggaca

SEQ ID NO: 46 - DNA, 37 bp, Artificial Sequence
  synthetic oligonucleotide
ggcgcgagatcttgcggcccgggcggtcgccgggatc

SEQ ID NO: 47 - protein, 50 aa, Streptomyces coelicolor
MQRRTRPTRTPGGLPLMSRVEQPSRRALLAAAVAAAAVTGGAIPATARAA

SEQ ID NO: 48 - DNA, 30 bp, Artificial Sequence
  synthetic oligonucleotide
ggcgcgcatatgggaacgcaggagtcggac

SEQ ID NO: 49 - DNA, 34 bp, Artificial Sequence
  synthetic oligonucleotide
ggcgcgggatccggtgccggcggctctcgccgtg

SEQ ID NO: 50 - protein, 43 aa, Streptomyces coelicolor
MGTQESDERAGGGTGRRALLGAAVLGAGGAVLGLPGTARAAGT

SEQ ID NO: 51 - DNA, 57 bp, Artificial Sequence
  synthetic oligonucleotide
ggcggccatatgggcggtgtctcgcgccgtgccttcacggtggcggcgttgtcggcg

SEQ ID NO: 52 - DNA, 60 bp, Artificial Sequence
  synthetic oligonucleotide
ggcggcggatcccgccgacgcctcgggcacgagcgtgaacgccgacaacgccgccaccgt

SEQ ID NO: 53 - protein, 26 aa, Streptomyces coelicolor
MGGVSRRAFTVAALSAFTLVPEASAA

SEQ ID NO: 54 - DNA, 57 bp, Artificial Sequence; identical to SEQ ID NO: 51
  synthetic oligonucleotide

SEQ ID NO: 55 - DNA, 69 bp, Artificial Sequence
  synthetic oligonucleotide
ggcggcggatccgggggtcgccgccgacgcctcgggcacgagcgtgaacgccgacaacgccgccaccgt

SEQ ID NO: 56 - protein, 28 aa, Streptomyces coelicolor
MGGVSRRAFTVAALSAFTLVPEASAATP

SEQ ID NO: 57 - DNA, 89 bp, Artificial Sequence
  synthetic oligonucleotide
ggcgcgcatatgcgccgacgctcactcctcatcgccgtccccacgggactggtcacgctcgccgcctgcggtgacagcgacgactccgg

SEQ ID NO: 58 - DNA, 90 bp, Artificial Sequence
  synthetic oligonucleotide
ggcgcgggatcccgcggccgaggtggccgacgcgtcgggcgacgggctctcgctcgcgttgctcgacgagccggagtcgtcgctgtcacc

SEQ ID NO: 59 - protein, 46 aa, Streptomyces coelicolor
MRRRSLLIAVPTGLVTLAACGDSDDSGSSSNASESPSPDASATSAA

SEQ ID NO: 60 - DNA, 27 bp, Artificial Sequence
  synthetic oligonucleotide
ggcgcgcatatgaccgctcccctctcg

SEQ ID NO: 61 - DNA, 30 bp, Artificial Sequence
  synthetic oligonucleotide
ggcgcgggatccgggcgcggcctcggccgc

SEQ ID NO: 62 - protein, 42 aa, Streptomyces coelicolor
MTAPLSRHRRALAIPAGLAVAASLAFLPGTPAAATPAAEAAP

SEQ ID NO: 63 - DNA, 27 bp, Artificial Sequence
  synthetic oligonucleotide
ggcgcgcatatgtcgctcactcgcagg

SEQ ID NO: 64 - DNA, 31 bp, Artificial Sequence
  synthetic oligonucleotide
ggcgcgggatcccgtggacgcgagggcgttg

SEQ ID NO: 65 - protein, 38 aa, Streptomyces coelicolor
MSLTRRDFAGRSALTGAGVVLAGSVGALATAPNALAST

SEQ ID NO: 66 - DNA, 29 bp, Artificial Sequence
  synthetic oligonucleotide
ggcgcgcatatgagacgaagagcgagatc

SEQ ID NO: 67 - DNA, 31 bp, Artificial Sequence
  synthetic oligonucleotide
ggcgcgggatccgggttgtgcctgggcgatg

SEQ ID NO: 68 - protein, 29 aa, Streptomyces coelicolor
MRRRARSILAVGALLIGGASFAPIAQAQP

SEQ ID NO: 69 - DNA, 28 bp, Artificial Sequence
  synthetic oligonucleotide
ggcgcgcatatgcacgtgcgcgcagtag

SEQ ID NO: 70 - DNA, 33 bp, Artificial Sequence
  synthetic oligonucleotide
ggcgcgggatcccgcctcgtccgcccgcgcgtg

SEQ ID NO: 71 - protein, 30 aa, Streptomyces coelicolor
MHVRAVAVTTTALLGVALTAPLSHARADEA

SEQ ID NO: 72 - DNA, 29 bp, Artificial Sequence
  synthetic oligonucleotide
ggcgcgcatatgaccagtcgacacagagc

SEQ ID NO: 73 - DNA, 30 bp, Artificial Sequence
  synthetic oligonucleotide
ggcgcgggatccgggggccgcgtcggccgc

SEQ ID NO: 74 - protein, 46 aa, Streptomyces coelicolor
MTSRHRASENSRTPSRRTVVKAAAAGAVLAAPLAAALPAGAADAAP

SEQ ID NO: 75 - DNA, 29 bp, Artificial Sequence
  synthetic oligonucleotide
ggcgcgcatatgcctgccacgcgccgtac

SEQ ID NO: 76 - DNA, 33 bp, Artificial Sequence
  synthetic oligonucleotide
ggcgcgggatccggtgtcggcccgtgcggcgag

SEQ ID NO: 77 - protein, 35 aa, Streptomyces coelicolor
MPATRRTARVRRVAAVTVTALAAALLPPLAARADT

SEQ ID NO: 78 - DNA, 27 bp, Artificial Sequence
  synthetic oligonucleotide
ggcgcgcatatgacacccgcgaaccac

SEQ ID NO: 79 - DNA, 31 bp, Artificial Sequence
  synthetic oligonucleotide
ggcgcgggatccctcggcggcgctcgcggtg

SEQ ID NO: 80 - protein, 64 aa, Streptomyces coelicolor
MTPANHQAPTSAPSPAPSQSSHAPELRAAARSLGRRRFLTVTGAAAALAFAVNLPAAGTASAAE

SEQ ID NO: 81 - DNA, 32 bp, Artificial Sequence
  synthetic oligonucleotide
ggcgcgcatatgcgtctcagacgcccccgcag

SEQ ID NO: 82 - DNA, 31 bp, Artificial Sequence
  synthetic oligonucleotide
ggcgcgggatccgctgtcggcgacggcactg

SEQ ID NO: 83 - protein, 62 aa, Streptomyces coelicolor
MRLRRPRRPSDGPPGRSGARRLRVASAPLIPLTVIGAVVAGLIGPEPPSDGPLPGTSAVADS

SEQ ID NO: 84 - DNA, 63 bp, Artificial Sequence
  synthetic oligonucleotide
ggcgcgcatatgagcacaggaccagtcagacgcggggcggccctcctgtcggcggggctcgtg

SEQ ID NO: 85 - DNA, 69 bp, Artificial Sequence
  synthetic oligonucleotide
ggcgcgggatccctgcgccgccgaggtcccggccggcagcagcgcgatcacgagccccgccgacaggag

SEQ ID NO: 86 - protein, 30 aa, Streptomyces coelicolor
MSTGPVRRGAALLSAGLVIALLPAGTSAAQ

SEQ ID NO: 87 - DNA, 36 bp, Artificial Sequence
  synthetic oligonucleotide
ggcgcgcatatgttcacatcacgcgcgcgcctcaga

SEQ ID NO: 88 - DNA, 32 bp, Artificial Sequence
  synthetic oligonucleotide
ggcgcgggatccgtcggcggcgacggcgggag

SEQ ID NO: 89 - protein, 37 aa, Streptomyces coelicolor
MFTSRARLRTRRSRLLTCTALAVAAGMLVTTPAVAAD

SEQ ID NO: 90 - DNA, 27 bp, Artificial Sequence
  synthetic oligonucleotide
ggcgcgcatatgaccaacagcccccag

SEQ ID NO: 91 - DNA, 30 bp, Artificial Sequence
  synthetic oligonucleotide
ggcgcgggatccgtcggcggcgagggccac

SEQ ID NO: 92 - protein, 42 aa, Streptomyces coelicolor
MTNSPQREPIPGARRAARLALATGLAAALAAVGPVPVALAAD

SEQ ID NO: 93 - DNA, 28 bp, Artificial Sequence
  synthetic oligonucleotide
ggcgcgcatatgcaccacagcagcacgg

SEQ ID NO: 94 - DNA, 32 bp, Artificial Sequence
  synthetic oligonucleotide
ggcgcgggatccggcgtcggcgtaggccgtac

SEQ ID NO: 95 - protein, 42 aa, Streptomyces coelicolor
MHHSSTAGTGSTAQPSRRSVLTATAVVTAALAAGGGTAYADA

SEQ ID NO: 96 - DNA, 29 bp, Artificial Sequence
  synthetic oligonucleotide
ggcgcgcatatgtcccacgccagcgctac

SEQ ID NO: 97 - DNA, 31 bp, Artificial Sequence
  synthetic oligonucleotide
ggcgcgggatccgtcgcccgccccatcgctc

SEQ ID NO: 98 - protein, 42 aa, Streptomyces coelicolor
MSHASATHPTRRGILAAGGALGLGAVLAACGDGDGKSDGAGD

SEQ ID NO: 99 - DNA, 30 bp, Artificial Sequence
  synthetic oligonucleotide
ggcgcgcatatgagacctcatcgacggcac

SEQ ID NO: 100 - DNA, 31 bp, Artificial Sequence
  synthetic oligonucleotide
ggcgcgggatcccggttccgcggccgccttc

SEQ ID NO: 101 - protein, 44 aa, Streptomyces coelicolor
MRPHRRHHRTTPRITRLLGSLLLVAAVGAMTTGAAPVRKAAAEP

SEQ ID NO: 102 - DNA, 30 bp, Artificial Sequence
  synthetic oligonucleotide
ggcgcgcatatgagacgaccagtcgcgctg

SEQ ID NO: 103 - DNA, 33 bp, Artificial Sequence
  synthetic oligonucleotide
ggcgcgggatccgccggccgctgccgccgcctc

SEQ ID NO: 104 - protein, 36 aa, Streptomyces coelicolor
MRRPVALRLQAALGTLALAAAAGVVLTMPEAAAAAG

SEQ ID NO: 105 - DNA, 69 bp, Artificial Sequence
  synthetic oligonucleotide
ggcgcgcatatgtccagattccgttcacgcgccgcggtcaccgcgggtctgacggccatcgcggtggcc

SEQ ID NO: 106 - DNA, 72 bp, Artificial Sequence
  synthetic oligonucleotide
ggcgcgggatccggtggcggcctggacgggtccgacgacgaggcagccggcggccaccgcgatggccgtcag

SEQ ID NO: 107 - protein, 33 aa, Streptomyces coelicolor
MSRFRSRAAVTAGLTAIAVAAGCLVVGPVQAAT

SEQ ID NO: 108 - DNA, 27 bp, Artificial Sequence
  synthetic oligonucleotide
ggcgcgcatatgcggcacagcggacgg

SEQ ID NO: 109 - DNA, 30 bp, Artificial Sequence
  synthetic oligonucleotide
ggcgcgggatccctcgacccccgtggtatc

SEQ ID NO: 110 - protein, 37 aa, Streptomyces coelicolor
MRHSGRRVKRATVLAAGAAVAGLLTAACSSPDTTGVE

SEQ ID NO: 111 - DNA, 64 bp, Artificial Sequence
  synthetic oligonucleotide
ggccggcatatggtcaaccgacgtgatctcatcaagtggagtgccgtcgcactcggagcgggtg

SEQ ID NO: 112 - DNA, 76 bp, Artificial Sequence
  synthetic oligonucleotide
ggccggggatccgaggtctgcggcatgagcggcgggtgcgggacccgcgagccccgcacccgctccgagtgcgacg

SEQ ID NO: 113 - protein, 33 aa, Streptomyces coelicolor
MVNRRDLIKWSAVALGAGAGLAGPAPAAHAADL

SEQ ID NO: 114 - DNA, 27 bp, Artificial Sequence
  synthetic oligonucleotide
ggcgcgcatatgaccaagcctgttgtg

SEQ ID NO: 115 - DNA, 31 bp, Artificial Sequence
  synthetic oligonucleotide
ggcgcgggatccaccggagccttgggacgag

SEQ ID NO: 116 - DNA, 30 bp, Artificial Sequence
  synthetic oligonucleotide
ggcgcgcatatgcgcaagctcctgccgttg

SEQ ID NO: 117 - DNA, 31 bp, Artificial Sequence
  synthetic oligonucleotide
ggcgcgggatccgggcgcggcctgcgccgac

SEQ ID NO: 118 - protein, 87 aa, Streptomyces coelicolor
MRKLLPLIGTPSGSHPGGRSAMTCRFRCGDACFHEVPNTSSNEYVGDVIAGALSRRSMMRAAAVVTVAAAGAGAVGVAGAPSAQAAP

SEQ ID NO: 119 - DNA, 31 bp, Artificial Sequence
  synthetic oligonucleotide
ggcgcgcatatgccgaacaccagctccaacg

SEQ ID NO: 120 - DNA, 31 bp, Artificial Sequence; identical to SEQ ID NO: 117
  synthetic oligonucleotide

SEQ ID NO: 121 - protein, 52 aa, Streptomyces coelicolor
MPNTSSNEYVGDVIAGALSRRSMMRAAAVVTVAAAGAGAVGVAGAPSAQAAP

SEQ ID NO: 122 - DNA, 31 bp, Artificial Sequence
  synthetic oligonucleotide
ggcgcgcatatgaaccggcgggccctcgctc

SEQ ID NO: 123 - DNA, 31 bp, Artificial Sequence
  synthetic oligonucleotide
ggcgcgggatccgctgttggcggcggcggag

SEQ ID NO: 124 - protein, 42 aa, Streptomyces coelicolor
MNRRALALGALAVSGALALTACGSDDTGGNSGSDSSSAAANS

SEQ ID NO: 125 - DNA, 27 bp, Artificial Sequence
  synthetic oligonucleotide
ggcgcgcatatgccagtcacagcacag

SEQ ID NO: 126 - DNA, 31 bp, Artificial Sequence
  synthetic oligonucleotide
ggcgcgggatcccccgcccgcgctcgccgag

SEQ ID NO: 127 - protein, 45 aa, Streptomyces coelicolor
MPVTAQPHQPRRRRRTSRLLVVAATVVTAGVLAAAALPASASAGG

SEQ ID NO: 128 - DNA, 27 bp, Artificial Sequence
  synthetic oligonucleotide
ggcgcgcatatgacccacacctcacgg

SEQ ID NO: 129 - DNA, 30 bp, Artificial Sequence
  synthetic oligonucleotide
ggcgcgggatcccggagcggcggacgcagc

SEQ ID NO: 130 - protein, 50 aa, Streptomyces coelicolor
MTHTSRRGVLAAFAASAATVPFGGAAAASPARATATAASAVDPAAASAAP

SEQ ID NO: 131 - DNA, 28 bp, Artificial Sequence
  synthetic oligonucleotide
ggcgcgcatatgcgtcggacatcaagac

SEQ ID NO: 132 - DNA, 34 bp, Artificial Sequence
  synthetic oligonucleotide
ggcgcgggatccgtcgtcgccgctctcgccgctg

SEQ ID NO: 133 - protein, 34 aa, Streptomyces coelicolor
MRRTSRLIRVAVGVASLALAATACGGTSGESGDD

SEQ ID NO: 134 - DNA, 30 bp, Artificial Sequence
  synthetic oligonucleotide
ggcgcgcatatgcgccggatttcccggatc

SEQ ID NO: 135 - DNA, 30 bp, Artificial Sequence
  synthetic oligonucleotide
ggcgcgggatccggactccgaaccggcgtc

SEQ ID NO: 136 - protein, 35 aa, Streptomyces coelicolor
MRRISRITVAGAATASLALALAACGGTSTDSGSES

SEQ ID NO: 137 - DNA, 27 bp, Artificial Sequence
  synthetic oligonucleotide
ggccgccatatgacctcgtcgctgcac

SEQ ID NO: 138 - DNA, 30 bp, Artificial Sequence
  synthetic oligonucleotide
ggccgcggatccgacgctcgcggcgtgcgc

SEQ ID NO: 139 - protein, 34 aa, Streptomyces coelicolor
MTSSLHHAIRLTTASAIALGGLVTLGTSAHAASV

SEQ ID NO: 140 - DNA, 72 bp, Artificial Sequence
  synthetic oligonucleotide
ggcggccatatgagcattctccgtaaccgcaccgccacggccgccatagtcgcggtcgcagccggcgccctg

SEQ ID NO: 141 - DNA, 75 bp, Artificial Sequence
  synthetic oligonucleotide
ggcggcggatccggcggagctgtcgctgtcgccaccaccgcaggcggtgagggtcagggcgccggctgcgaccgc

SEQ ID NO: 142 - protein, 35 aa, Streptomyces coelicolor
MSILRNRTATAAIVAVAAGALTLTACGGGDSDSSA

SEQ ID NO: 143 - DNA, 28 bp, Artificial Sequence
  synthetic oligonucleotide
ggccgccatatgttgagcagcagcactc

SEQ ID NO: 144 - DNA, 30 bp, Artificial Sequence
  synthetic oligonucleotide
ggccgcggatccgtcgggggccgcggtggc

SEQ ID NO: 145 - protein, 53 aa, Streptomyces coelicolor
MLSSSTPHRRTSHTTSHTSRRTAHRRAAAVALAGVAALIATAVQSGTATAAPD

SEQ ID NO: 146 - DNA, 28 bp, Artificial Sequence
  synthetic oligonucleotide
ggcgcgcatatgatcaccactagtctgc

SEQ ID NO: 147 - DNA, 30 bp, Artificial Sequence
  synthetic oligonucleotide
ggcgcgggatcccggggcggccttggcggg

SEQ ID NO: 148 - protein, 44 aa, Streptomyces coelicolor
MITTSLRRRTAAAVLSLSAVLATTAATAPGAAPAPSAAPAKAAP

SEQ ID NO: 149 - DNA, 64 bp, Artificial Sequence
  synthetic oligonucleotide
ggcgcgcatatgcgtgcccgtagccatgcagttctcgccgcctcggtcgtcctcgccctggccg

SEQ ID NO: 150 - DNA, 67 bp, Artificial Sequence
  synthetic oligonucleotide
ggcgcgggatcccggcgcggcgaaggcgggggccgccagcgggccggcggccagggcgaggacgacc

SEQ ID NO: 151 - protein, 30 aa, Streptomyces coelicolor
MRARSHAVLAASVVLALAAGPLAAPAFAAP

SEQ ID NO: 152 - DNA, 28 bp, Artificial Sequence
  synthetic oligonucleotide
ggcgcgcatatgaacacgcgtatgcgtc

SEQ ID NO: 153 - DNA, 31 bp, Artificial Sequence
  synthetic oligonucleotide
ggcgcgggatccctccttggcgctgccacag

SEQ ID NO: 154 - protein, 29 aa, Streptomyces coelicolor
MNTRMRRAAVAVATTAMAVSLAACGSAKE

SEQ ID NO: 155 - DNA, 28 bp, Artificial Sequence
  synthetic oligonucleotide
ggcgcgcatatgtccacgtcccgcaacg

SEQ ID NO: 156 - DNA, 31 bp, Artificial Sequence
  synthetic oligonucleotide
ggcgcgggatccgggctgggcggcggcggtg

SEQ ID NO: 157 - protein, 42 aa, Streptomyces coelicolor
MSTSRNAATRRQVLARTGALGAGIAFTGALSELFAGTAAAQP

SEQ ID NO: 158 - DNA, 74 bp, Artificial Sequence
  synthetic oligonucleotide
ggcgcgcatatggacaccacttactggagcagacgtcgcgtcctcaccgtgctcggcgccgccaccgcggcgac

SEQ ID NO: 159 - DNA, 72 bp, Artificial Sequence
  synthetic oligonucleotide
ggcgcgggatccggcggcgagagcgcgggacggggcggcgagagggatcgaggtcgccgcggtggcggcgcc

SEQ ID NO: 160 - protein, 35 aa, Streptomyces coelicolor
MDTTYWSRRRVLTVLGAATAATSIPLAAPSRALAA

SEQ ID NO: 161 - DNA, 76 bp, Artificial Sequence
  synthetic oligonucleotide
ggcgcgcatatgcaccgcacaagacccgggggccacccccgatccatacgcttcgccacactcgcgatctccctga

SEQ ID NO: 162 - DNA, 79 bp, Artificial Sequence
  synthetic oligonucleotide
ggcgcgggatccggtgacggcgacggccggtgacgccgtcatgagcgcggagcccgcggtcagggagatcgcgagtgtg

SEQ ID NO: 163 - protein, 38 aa, Streptomyces coelicolor
MHRTRPGGHPRSIRFATLAISLTAGSALMTASPAVAVT

SEQ ID NO: 164 - DNA, 27 bp, Artificial Sequence
  synthetic oligonucleotide
ggcgcgcatatgaccgaggtgtcccgc

SEQ ID NO: 165 - DNA, 30 bp, Artificial Sequence
  synthetic oligonucleotide
ggcgcgggatccccctgccgcggccgctgc

SEQ ID NO: 166 - protein, 50 aa, Streptomyces coelicolor
MTEVSRRKLMKGAAVSGGALALPALGAPPATAAPAAGPEDLPGPAAAAAG

SEQ ID NO: 167 - DNA, 27 bp, Artificial Sequence
  synthetic oligonucleotide
ggcgcgcatatgtccgccatttctcgc

SEQ ID NO: 168 - DNA, 32 bp, Artificial Sequence
  synthetic oligonucleotide
ggcgcgggatccggcgatcgccgcggcgggtg

SEQ ID NO: 169 - protein, 45 aa, Streptomyces coelicolor
MSAISRRRFIRHGAVAGGAAVALPALGGWAGEAFAAQPSPAAAIA

SEQ ID NO: 170 - DNA, 27 bp, Artificial Sequence
  synthetic oligonucleotide
ggcgcgcatatggacagacgagccctc

SEQ ID NO: 171 - DNA, 30 bp, Artificial Sequence
  synthetic oligonucleotide
ggcgcgggatccggtcgcggcgtccgtccc

SEQ ID NO: 172 - protein, 37 aa, Streptomyces coelicolor
MDRRALMLATGGLLGAAGAAQLTAAPSAVAAGTDAAT

SEQ ID NO: 173 - DNA, 29 bp, Artificial Sequence
  synthetic oligonucleotide
ggcgcgcatatgccgcactcgcccgtgtc

SEQ ID NO: 174 - DNA, 36 bp, Artificial Sequence
  synthetic oligonucleotide
ggcgcgggatccctcgtcggccgccgcccgccgtac

SEQ ID NO: 175 - protein, 59 aa, Streptomyces coelicolor
MPHSPVSPAESPAPQPGRPRPVVSRRRLLEGGAAVLGALALSASPLTAQAAVRRAAADE

SEQ ID NO: 176 - DNA, 27 bp, Artificial Sequence
  synthetic oligonucleotide
ggccgccatatgaccccgttcaccgac

SEQ ID NO: 177 - DNA, 30 bp, Artificial Sequence
  synthetic oligonucleotide
ggccgcggatccccggtcgtgggcgagcgc

SEQ ID NO: 178 - protein, 63 aa, Streptomyces coelicolor
MTPFTDSSRTDAGTDPSADGPGESLRRALGVNRRRFLSTCTAVAAGAVAAPVFGASPALAHDR

SEQ ID NO: 179 - DNA, 27 bp, Artificial Sequence
  synthetic oligonucleotide
ggcgcgcatatgaaccggcgccgcttc

SEQ ID NO: 180 - DNA, 27 bp, Artificial Sequence
  synthetic oligonucleotide
ggcgcgcatatgaaccggcgccgcttc

SEQ ID NO: 181 - protein, 33 aa, Streptomyces coelicolor
MNRRRFLSTCTAVAAGAVAAPVFGASPALAHDR

SEQ ID NO: 182 - DNA, 26 bp, Artificial Sequence
  synthetic oligonucleotide
gcgcgcatatgccgtcttcccccacg

SEQ ID NO: 183 - DNA, 32 bp, Artificial Sequence
  synthetic oligonucleotide
ggcgcgggatccggagtcggcggcggcggttc

SEQ ID NO: 184 - protein, 48 aa, Streptomyces coelicolor
MPSSPTPSPTPSGAPEPSGVRRRSLLAAAAAAPVLASLAGAGTAAADS

SEQ ID NO: 185 - DNA, 27 bp, Artificial Sequence
  synthetic oligonucleotide
ggcgcgcatatgcgctcccaccgccgc

SEQ ID NO: 186 - DNA, 31 bp, Artificial Sequence
  synthetic oligonucleotide
ggcgcgggatccaccgtcaccggacgcggaa

SEQ ID NO: 187 - protein, 36 aa, Streptomyces coelicolor
MRSHRRPRLLAPFLLVPLMAGCFASGGGDDSASGDG

SEQ ID NO: 188 - DNA, 27 bp, Artificial Sequence
  synthetic oligonucleotide
ggcgcgcatatggccgaagtgaaccgg

SEQ ID NO: 189 - DNA, 33 bp, Artificial Sequence
  synthetic oligonucleotide
ggcgcgggatcccgggagtgccgcggcccggtc

SEQ ID NO: 190 - protein, 33 aa, Streptomyces coelicolor
MAEVNRRRFLQLAGATTAFSALSASIDRAAALP

SEQ ID NO: 191 - DNA, 27 bp, Artificial Sequence
  synthetic oligonucleotide
ggccgccatatgaaccgcctgcgcacc

SEQ ID NO: 192 - DNA, 30 bp, Artificial Sequence
  synthetic oligonucleotide
ggccgcggatccggagccgtcggcgtgggc

SEQ ID NO: 193 - protein, 33 aa, Streptomyces coelicolor
MNRLRTLTATTAALAASLLVPSLLTAPAHADGS

SEQ ID NO: 194 - DNA, 27 bp, Artificial Sequence
  synthetic oligonucleotide
ggcgcgcatatgagacgcctcctgctc

SEQ ID NO: 195 - DNA, 30 bp, Artificial Sequence
  synthetic oligonucleotide
ggcgcgggatcccttgtcggcggcgggttc

SEQ ID NO: 196 - protein, 30 aa, Streptomyces coelicolor
MRRLLLTAAATTAAALTLAACGTTEPAADK

SEQ ID NO: 197 - DNA, 27 bp, Artificial Sequence
  synthetic oligonucleotide
ggcgcgcatatgagcacagaggaacag

SEQ ID NO: 198 - DNA, 33 bp, Artificial Sequence
  synthetic oligonucleotide
ggcgcgggatccgccggccgcgtcggccgcctg

SEQ ID NO: 199 - protein, 47 aa, Streptomyces coelicolor
MSTEEQSSGRRHPDRRVLLRAAVAVPAAGAAVAGTAAVPAQAADAAG

SEQ ID NO: 200 - DNA, 28 bp, Artificial Sequence
  synthetic oligonucleotide
ggccgccatatgtccagacagatcgatc

SEQ ID NO: 201 - DNA, 31 bp, Artificial Sequence
  synthetic oligonucleotide
ggccgcggatcctccggggtttccggcggag

SEQ ID NO: 202 - protein, 43 aa, Streptomyces coelicolor
MSRQIDRRSFLRRGAAGAAALAVGPGLLAACSTDEPGSAGNPG

SEQ ID NO: 203 - protein, 29 aa, Streptomyces coelicolor
MRRIRLLAAISAGLALAAGAVAPVPALAS

SEQ ID NO: 204 - protein, 40 aa, Streptomyces coelicolor
MEPTVRTHPTRRLPTALVLTAALLATGCSEQSDDGREPAA

SEQ ID NO: 205 - protein, 37 aa, Streptomyces coelicolor
MERERTAPVPTRRRLLKGAALATVPYTLLSGTRAAAQ

SEQ ID NO: 206 - protein, 31 aa, Streptomyces coelicolor
MALTTRRRALTTLGAALTGAVALPAGTALAS

SEQ ID NO: 207 - protein, 43 aa, Streptomyces coelicolor
MTSAPFHPAAGPARRTVVAAAGAAGLTAVLAACSDSDDGASGD

SEQ ID NO: 208 - protein, 51 aa, Streptomyces coelicolor
MQRRTRPTRTPGGLPLMSRVEQPSRRALLAAAVAAAAVTGGAIPATARAAS

SEQ ID NO: 209 - protein, 41 aa, Streptomyces coelicolor
MGTQESDERAGGGTGRRALLGAAVLGAGGAVLGLPGTARAA

SEQ ID NO: 210 - protein, 26 aa, Streptomyces coelicolor
MGGVSRRAFTVAALSAFTLVPEASAA

SEQ ID NO: 211 - protein, 46 aa, Streptomyces coelicolor; identical to SEQ ID NO: 59

SEQ ID NO: 212 - protein, 41 aa, Streptomyces coelicolor
MTAPLSRHRRALAIPAGLAVAASLAFLPGTPAAATPAAEAA

SEQ ID NO: 213 - protein, 37 aa, Streptomyces coelicolor
MSLTRRDFAGRSALTGAGVVLAGSVGALATAPNALAS

SEQ ID NO: 214 - protein, 28 aa, Streptomyces coelicolor
MRRRARSILAVGALLIGGASFAPIAQAQ

SEQ ID NO: 215 - protein, 46 aa, Streptomyces coelicolor; identical to SEQ ID NO: 74

SEQ ID NO: 216 - protein, 34 aa, Streptomyces coelicolor
MPATRRTARVRRVAAVTVTALAAALLPPLAARAD

SEQ ID NO: 217 - protein, 63 aa, Streptomyces coelicolor
MTPANHQAPTSAPSPAPSQSSHAPELRAAARSLGRRRFLTVTGAAAALAFAVNLPAAGTASAA

SEQ ID NO: 218 - protein, 61 aa, Streptomyces coelicolor
MRLRRPRRPSDGPPGRSGARRLRVASAPLIPLTVIGAVVAGLIGPEPPSDGPLPGTSAVAD

SEQ ID NO: 219 - protein, 41 aa, Streptomyces coelicolor
MTNSPQREPIPGARRAARLALATGLAAALAAVGPVPVALAA

SEQ ID NO: 220 - protein, 41 aa, Streptomyces coelicolor
MHHSSTAGTGSTAQPSRRSVLTATAVVTAALAAGGGTAYAD

SEQ ID NO: 221 - protein, 40 aa, Streptomyces coelicolor
MSHASATHPTRRGILAAGGALGLGAVLAACGDGDGKSDGA

SEQ ID NO: 222 - protein, 43 aa, Streptomyces coelicolor
MRPHRRHHRTTPRITRLLGSLLLVAAVGAMTTGAAPVRKAAAE

SEQ ID NO: 223 - protein, 35 aa, Streptomyces coelicolor
MRRPVALRLQAALGTLALAAAAGVVLTMPEAAAAA

SEQ ID NO: 224 - protein, 36 aa, Streptomyces coelicolor
MRHSGRRVKRATVLAAGAAVAGLLTAACSSPDTTGV

SEQ ID NO: 225 - protein, 41 aa, Streptomyces coelicolor
MTKPVVPSGVSRRGFLGGSLGVAGAVLLAACSGGGNSSQGS

SEQ ID NO: 226 - protein, 86 aa, Streptomyces coelicolor
MRKLLPLIGTPSGSHPGGRSAMTCRFRCGDACFHEVPNTSSNEYVGDVIAGALSRRSMMRAAAVVTVAAAGAGAVGVAGAPSAQAA

SEQ ID NO: 227 - protein, 41 aa, Streptomyces coelicolor
MNRRALALGALAVSGALALTACGSDDTGGNSGSDSSSAAAN

SEQ ID NO: 228 - protein, 44 aa, Streptomyces coelicolor
MPVTAQPHQPRRRRRTSRLLVVAATVVTAGVLAAAALPASASAG

SEQ ID NO: 229 - protein, 49 aa, Streptomyces coelicolor
MTHTSRRGVLAAFAASAATVPFGGAAAASPARATATAASAVDPAAASAA

SEQ ID NO: 230 - protein, 33 aa, Streptomyces coelicolor
MRRTSRLIRVAVGVASLALAATACGGTSGESGD

SEQ ID NO: 231 - protein, 34 aa, Streptomyces coelicolor
MRRISRITVAGAATASLALALAACGGTSTDSGSE

SEQ ID NO: 232 - protein, 43 aa, Streptomyces coelicolor
MITTSLRRRTAAAVLSLSAVLATTAATAPGAAPAPSAAPAKAA

SEQ ID NO: 233 - protein, 28 aa, Streptomyces coelicolor
MNTRMRRAAVAVATTAMAVSLAACGSAK

SEQ ID NO: 234 - protein, 41 aa, Streptomyces coelicolor
MSTSRNAATRRQVLARTGALGAGIAFTGALSELFAGTAAAQ

SEQ ID NO: 235 - protein, 35 aa, Streptomyces coelicolor; identical to SEQ ID NO: 160

SEQ ID NO: 236 - protein, 50 aa, Streptomyces coelicolor; identical to SEQ ID NO: 166

SEQ ID NO: 237 - protein, 36 aa, Streptomyces coelicolor
MSAISRRRFIRHGAVAGGAAVALPALGGWAGEAFAA

SEQ ID NO: 238 - protein, 31 aa, Streptomyces coelicolor
MDRRALMLATGGLLGAAGAAQLTAAPSAVAA

SEQ ID NO: 239 - protein, 58 aa, Streptomyces coelicolor
MPHSPVSPAESPAPQPGRPRPVVSRRRLLEGGAAVLGALALSASPLTAQAAVRRAAAD

SEQ ID NO: 240 - protein, 61 aa, Streptomyces coelicolor
MTPFTDSSRTDAGTDPSADGPGESLRRALGVNRRRFLSTCTAVAAGAVAAPVFGASPALAH

SEQ ID NO: 241 - protein, 47 aa, Streptomyces coelicolor
MPSSPTPSPTPSGAPEPSGVRRRSLLAAAAAAPVLASLAGAGTAAAD

SEQ ID NO: 242 - protein, 35 aa, Streptomyces coelicolor
MRSHRRPRLLAPFLLVPLMAGCFASGGGDDSASGD

SEQ ID NO: 243 - protein, 32 aa, Streptomyces coelicolor
MAEVNRRRFLQLAGATTAFSALSASIDRAAAL

SEQ ID NO: 244 - protein, 29 aa, Streptomyces coelicolor
MRRLLLTAAATTAAALTLAACGTTEPAAD

SEQ ID NO: 245 - protein, 46 aa, Streptomyces coelicolor
MSTEEQSSGRRHPDRRVLLRAAVAVPAAGAAVAGTAAVPAQAADAA

SEQ ID NO: 246 - protein, 42 aa, Streptomyces coelicolor
MKTSWRSASLVAGAAALLALTTACGQDGGGPTGSQNVGATAA

SEQ ID NO: 247 - protein, 36 aa, Streptomyces coelicolor
MTRLSAALRGLATTFAALLAVTAVPAASAPATAQAR

SEQ ID NO: 248 - protein, 28 aa, Streptomyces coelicolor
MHVRAVAVTTTALLGVALTAPLSHARAD

SEQ ID NO: 249 - protein, 38 aa, Streptomyces coelicolor
MKASRIAAVGAVSGIAVLALSAPAFAHVSVQPEGAAAK

SEQ ID NO: 250 - protein, 38 aa, Streptomyces coelicolor
MFQVTRTPAARLLGAAALSAAALASVCAAPSAAVAGAG

SEQ ID NO: 251 - protein, 32 aa, Streptomyces coelicolor
MTSSLHHAIRLTTASAIALGGLVTLGTSAHAA

SEQ ID NO: 252 - protein, 31 aa, Streptomyces coelicolor
MNRLRTLTATTAALAASLLVPSLLTAPAHAD

SEQ ID NO: 253 - protein, 26 aa, Artificial
  constructed mutant tatC strain
MFRRTLPVLAAAVGLAAAAAGPATAD

SEQ ID NO: 254 - protein, 62 aa, Artificial
  constructed mutant tatC strain
MRIAVIGAGGVGGYFGARLAAAGNEVTFVARGGHLAAIRRSGLVVHSPLGELRTSPDSVVAS

SEQ ID NO: 255 - protein, 36 aa, Artificial
  constructed mutant tatC strain
MLLRTTRTKPWRRLAAALSAAALGVGLLAGCGSDSD

SEQ ID NO: 256 - protein, 33 aa, Artificial
  constructed mutant tatC strain
MGDIRRRGAVALGVTALVAPLTLALTAAPAQAA

SEQ ID NO: 257 - protein, 37 aa, Artificial
  constructed mutant tatC strain
LRKSSIRRRATAFGTAGALVTATLIAGAVSAPAASAA

SEQ ID NO: 258 - protein, 30 aa, Artificial
  constructed mutant tatC strain
MRRFRLVGFLSSLVLAAGAALTGAATAQAA

SEQ ID NO: 259 - protein, 55 aa, Artificial
  constructed mutant tatC strain
MSLMRAPHACLTGGPTTLNGNTFRMPARRLATVAAATALAAGPATLAGAGSAHAT

SEQ ID NO: 260 - protein, 50 aa, Artificial
  constructed mutant tatC strain
LSAEHRRNRNKKRLLVYGVASAVVVAATTGTLALASPGLLGLDADQAAAA

SEQ ID NO: 261 - protein, 41 aa, Artificial
  constructed mutant tatC strain
MKKLSARRRHPLAALVVLLLALACTGGLYAAFAPASKAQAD

SEQ ID NO: 262 - protein, 33 aa, Artificial
  constructed mutant tatC strain
MGRNTRKRRTPLATKIVAGAAALAIGGGGLVWN

SEQ ID NO: 263 - protein, 38 aa, Artificial
  constructed mutant tatC strain
MPVRRRRPTALAAAVATAAALGTTALAALGGATAASAA

SEQ ID NO: 264 - protein, 30 aa, Artificial; identical to SEQ ID NO: 86
  constructed mutant tatC strain

SEQ ID NO: 265 - protein, 34 aa, Artificial
  constructed mutant tatC strain
MNVRRRRISGIAVTAATALGLGTLSACSSDSSAA

SEQ ID NO: 266 - protein, 36 aa, Artificial
  constructed mutant tatC strain
LFTSRARLRTRRSRLLTCTALAVAAGMLVTTPAVAA

SEQ ID NO: 267 - protein, 33 aa, Artificial
  constructed mutant tatC strain
MTRRSWTFRTAATTVAFAAAAATFSAAGVAQAD

SEQ ID NO: 268 - protein, 48 aa, Artificial
  constructed mutant tatC strain
MDTRRTHRRTRTGGTRFRATLLTAALLATACSAGGASTSAGSPAAKAA

SEQ ID NO: 269 - protein, 34 aa, Artificial
  constructed mutant tatC strain
MSSRARRIAISVAASLMTFSLASCGMLGASESSD

SEQ ID NO: 270 - protein, 49 aa, Artificial
  constructed mutant tatC strain
MAPIDSDDNTTAGAGEELRAGRRKAARYVVPVAVMGVAAATIGLVPALA

SEQ ID NO: 271 - protein, 27 aa, Artificial
  constructed mutant tatC strain
MNRRRPTLLAALTLTAAAALTLSACGS

SEQ ID NO: 272 - protein, 41 aa, Artificial
  constructed mutant tatC strain
MTARSTRRTTAAHSRLAAVGAIAVAGALLLTGCGDQTENGD

SEQ ID NO: 273 - protein, 51 aa, Artificial
  constructed mutant tatC strain
MLSSSTPHRRTSHTTSHTSRRTAHRRAAAVALAGVAALIATAVQSGTATAA

SEQ ID NO: 274 - protein, 40 aa, Artificial
  constructed mutant tatC strain
MTTQRTSGRRKQALAAAAVVAALLTTAACGGGGDDDSGGG

SEQ ID NO: 275 - protein, 39 aa, Artificial
  constructed mutant tatC strain
MAVLSIISRTASRRGVRALGVVAASAALALSASGNALAC

SEQ ID NO: 276 - protein, 32 aa, Artificial
  constructed mutant tatC strain
MPATSALRRTLAVIAALPLLTLAACGYGSQAK

SEQ ID NO: 277 - protein, 48 aa, Artificial
  constructed mutant tatC strain
MATLIPKKGSTLLNKGIRTRRARGALAGGTVLTAAAALLTAVPAAQAI

SEQ ID NO: 278 - protein, 46 aa, Artificial
  constructed mutant tatC strain
MSTTSVNGSRSKRRVLLRRALPVCVAAGVASIVVFGSGSSPDAAAR

SEQ ID NO: 279 - protein, 27 aa, Artificial
  constructed mutant tatC strain
MRRPVARNLTVLAVSAAVVVSVGAAAP

SEQ ID NO: 280 - protein, 63 aa, Artificial
  constructed mutant tatC strain
MRSVGAGRWRRVASQVGRSVTVWAVSTLTMLVLAGILPDFRLQSPDGDSATDIAMTAAVGAGA

SEQ ID NO: 281 - protein, 41 aa, Artificial
  constructed mutant tatC strain
MSSIRRSRSAVLGAVLTAGSLALSTAAAVAVTGGTPVADSD

SEQ ID NO: 282 - protein, 32 aa, Artificial
  constructed mutant tatC strain
MKLRKRRLCAATGVVLGALTALAALPPAAVAS

SEQ ID NO: 283 - protein, 34 aa, Artificial
  constructed mutant tatC strain
MPLRLGAFVHRRFIVPGALGAASLMLAIPASAAA

SEQ ID NO: 284 - protein, 40 aa, Artificial
  constructed mutant tatC strain
MTRTRTTRLRRPLLTGSTAVAAMAVTAGLVAATAEPAKAA

SEQ ID NO: 285 - protein, 39 aa, Artificial
  constructed mutant tatC strain
MSRQIDRRSFLRRGAAGAAALAVGPGLLAACSTDEPGSA

SEQ ID NO: 286 - protein, 39 aa, Artificial
  constructed mutant tatC strain
MRSTGPVSRIGRSLAAVTATAAAGAVAALGLAASPAAAA

SEQ ID NO: 287 - protein, 31 aa, Artificial
  constructed mutant tatC strain
MSPKSKRTRAALAALVVAAGSLVTAGAARAE

SEQ ID NO: 288 - protein, 34 aa, Artificial
  constructed mutant tatC strain
LNTSIRNRAVTGTALALAVSAVLTACGGDTSSSD

SEQ ID NO: 289 - protein, 29 aa, Artificial
  constructed mutant tatC strain
MRNTARWAATLGLTATAVCGPLAGASLAS

SEQ ID NO: 290 - protein, 32 aa, Artificial
  constructed mutant tatC strain
MPGKGLIVRQRSLIAITAALAAGALTLTACGS

SEQ ID NO: 291 - protein, 37 aa, Artificial
  constructed mutant tatC strain
MMSGRARLAMCAWAATLMAACALLPLVEPATWILQAA

SEQ ID NO: 292 - protein, 29 aa, Artificial
  constructed mutant tatC strain
MPARLRALVTVLCAAVLAALLPAAAAHAE

SEQ ID NO: 293 - protein, 32 aa, Artificial
  constructed mutant tatC strain
MHSTTSRGAVLATLLALIGVFATYRALPAGAD

SEQ ID NO: 294 - protein, 41 aa, Artificial
  constructed mutant tatC strain
MTRGRDGGAGAPPTKHRALLAAIVTLIVAISAAIYAGASAD

SEQ ID NO: 295 - protein, 32 aa, Artificial
  constructed mutant tatC strain
MSRFRSRAAVTAGLTAIAVAAGCLVVGPVQAA

SEQ ID NO: 296 - protein, 35 aa, Artificial
  constructed mutant tatC strain
MRPSSLRSPWTRAAVAGAAAAGLAAAGLAGVAHAA

SEQ ID NO: 297 - protein, 46 aa, Artificial
  constructed mutant tatC strain
MTVHKRACTTPPPRASRSFRVRWPVLIAAACAGLVLATTSPPAVAA

SEQ ID NO: 298 - protein, 29 aa, Artificial
  constructed mutant tatC strain
MRKKTFATAALLAAACLTLSACGSGADSD

SEQ ID NO: 299 - protein, 48 aa, Artificial
  constructed mutant tatC strain
MQLLPSGRALTAGAVAVVTLMAGGSAAGAASAPVHPATPTGTAAAAAA

SEQ ID NO: 300 - protein, 31 aa, Artificial
  constructed mutant tatC strain
MRTRTKLTTVLASGTLAAGLLVGGASSASAG

SEQ ID NO: 301 - protein, 36 aa, Artificial
  constructed mutant tatC strain
MSILRNRTATAAIVAVAAGALTLTACGGGDSDSSAK

SEQ ID NO: 302 - protein, 23 aa, Artificial
  constructed mutant tatC strain
MKRKLIAAIGIAGMMVSIAACGG

SEQ ID NO: 303 - protein, 39 aa, Artificial
  constructed mutant tatC strain
MESQVRGGTRWKRFAVVMVPSVAATAAIGVALAQGALAA

SEQ ID NO: 304 - protein, 29 aa, Artificial
  constructed mutant tatC strain
MRARSHAVLAASVVLALAAGPLAAPAFAA

SEQ ID NO: 305 - protein, 25 aa, Artificial
  constructed mutant tatC strain
MKLRKVSAASAAVLALALTATACGG

SEQ ID NO: 306 - protein, 35 aa, Artificial
  constructed mutant tatC strain
MFAMPRSARLTALSAALLAGALASSSTPALATAGA

SEQ ID NO: 307 - protein, 44 aa, Artificial
  constructed mutant tatC strain
MHRTRPGGHPRSIRFATLAISLTAGSALMTASPAVAVTGPAAAD

SEQ ID NO: 308 - protein, 33 aa, Artificial
  constructed mutant tatC strain
MKKRSSFVRVLGAAATAGALAWAVLAQGNAGAA

SEQ ID NO: 309 - protein, 33 aa, Artificial
  constructed mutant tatC strain
MWAALLAGLLMVLGLQATSASGQPAKAPAAAAA

SEQ ID NO: 310 - protein, 23 aa, Artificial
  constructed mutant tatC strain
MALAGAVCAAALAALTAVPAQAH

SEQ ID NO: 311 - protein, 27 aa, Artificial
  constructed mutant tatC strain
MPALATAALLLPLLGAAPSAAAPTAAP

SEQ ID NO: 312 - protein, 32 aa, Streptomyces coelicolor
MVNRRDLIKWSAVALGAGAGLAGPAPAAHAAD